(19) United States
(12) Patent Application Publication
(10) Pub. No.: US 2019/0194683 A1
(43) Pub. Date: Jun. 27, 2019

(54) METHODS AND COMPOSITIONS FOR RESISTANCE TO CYST NEMATODE IN PLANTS

(71) Applicant: Wisconsin Alumni Research Foundation, Madison, WI (US)

(72) Inventors: Andrew Farmer Bent, Madison, WI (US); Adam Milton Bayless, Madison, WI (US); Ryan W. Zapotocny, Madison, WI (US)

(21) Appl. No.: 16/102,475

(22) Filed: Aug. 13, 2018

Related U.S. Application Data
(60) Provisional application No. 62/544,824, filed on Aug. 12, 2017.
(60) Provisional application No. 62/544,856, filed on Aug. 13, 2017.

(51) Int. Cl. C12N 15/82

(52) U.S. Cl. CPC C12N 15/8285

(57) ABSTRACT

The disclosure relates to methods and compositions for producing plants or plant cells that exhibit improved cyst nematode resistance.

This application claims priority to U.S. Provisional Application Nos. 62/544,856 and 62/544,824, the disclosures of which are explicitly incorporated by reference herein.

### STATEMENT REGARDING FEDERALLY SPONSORED RESEARCH

This invention was made with government support under 17-CRHF-0-6055 awarded by the USDA/NIFA. The government has certain rights in the invention.

### BACKGROUND

### Field of the Invention

The present disclosure provides methods and compositions for conferring or producing nematode resistance in a plant or plant cells, and nematode resistant plants or plant cells. The disclosure further provides methods for improving growth or survival of a plant cell containing one or more Rhg1 genes capable of conferring nematode resistance.

### Description of Related Art

Soybean cyst nematode (Heterodera glycines; SCN) is consistently the most damaging disease or pest of U.S. soybeans, one of the world's most important crops (Niblack et al., 2006, Annu Rev Phytopathol 44, 283-303; Jones et al., 2013, Mol Plant Pathol 14, 946-961; Mitchum, 2016, Mol Plant Pathol 5, 175-181; T. W. Allen, 2017, Soybean Yield Loss Estimates Due to Diseases in the United States and Ontario, Canada, from 2010 to 2014. Plant Health Research. doi:10.1094/PHP-RS-16-0066). Plant parasitic nematodes, including cyst nematodes, infest the roots of many valuable crops and establish elaborate feeding structures (Kyndt et al., 2013, Planta 238, 807-818). Cyst nematodes secrete a complex arsenal of effector molecules that modulate the host's physiology and promote fusion of neighboring host cells into a large unicellular feeding site, termed a syncytium (Gheysen and Mitchum, 2011, Curr Opin Plant Biol 14, 415-421; Hewezi and Baum, 2013, Mol Plant Microbe Interact 26, 9-16; Mitchum et al., 2013, New Phytologist 199, 879-894), with negative effects on the health and propagation of the involved plants.

A soybean locus, Rhg1 (Resistance to Heterodera glycines), has been widely used by soybean breeders and growers as the best available disease resistance locus to reduce damage caused by SCN (Concibido et al., 2004, Crop Science 44, 1121-1131; Mitchum, 2016, Id.). The complex Rhg1 locus on soybean chromosome 18 is a tandemly repeated block of four genes: Glyma.18G022400 (formerly Glyma18g02580), Glyma.18G022500 (formerly Glyma18g02590), Glyma.18G022600 (formerly Glyma18g02600) and Glyma.18G022700 (formerly Glyma18g02610), as well as the adjacent nucleotides that comprise the chromosomal segment containing the above genes, which is tandemly repeated in haplotypes that confer increased SCN resistance (Cook et al., 2012, Science 338, 1206-1209; U.S. Patent Application Publ. No. 2013-0305410 A1). (The 13-character gene names are from the Wm82.a1 genome assembly and Glyma 1.0 gene models (Schmutz et al., 2010, Nature 463, 178-183) and the more recent 15-character gene names are from the U.S. Department of Energy Joint Genome Institute Wm82.a2 soybean genome assembly and Glyma 2.0 gene model naming revision.) The relevant genes at the Rhg1 locus do not encode proteins widely associated with plant disease resistance. Instead, resistance is mediated by copy number variation of three disparate genes at the Rhg1 locus, one of which (Glyma.18G022500) encodes proteins with high similarity to known α-SNAP proteins (U.S. Patent Application Publ. No. 2013-0305410 A1; Mitchum et al., 2004, Mol Plant Pathol 5, 175-181; Jones and Dangl, 2006, Nature 444, 323-329; Dodds and Rathjen, 2010, Nat Rev Genet 11, 539-548; Cook et al., 2012, Science 338, 1206-1209; Cook et al., 2014, Plant Physiol 165, 630-647; Lee et al., 2015, Mol Ecol 24, 1774-1791).

Alpha-Soluble NSF Attachment Protein (α-SNAP or α-SNAP herein) is a ubiquitous housekeeping protein in plants and animals that facilitates cellular vesicular trafficking by mediating the disassembly and reuse of the four-protein bundles of SNARE proteins (soluble NSF attachment protein receptor proteins) that form when t-SNARE and v-SNARE proteins anneal during vesicle docking to target membranes (Jahn and Scheller, 2006, Nat Rev Mol Cell Biol 7, 631-643; Baker and Hughson, 2016, Nat Rev Mol Cell Biol 17, 465-479; Zhao and Brunger, 2016, J Mol Biol 428, 1912-1926). α-SNAP functions together with the ATPase N-ethylmaleimide Sensitive Factor (NSF) to carry out this SNARE bundle disassembly (Zhao and Brunger, 2015, J Mol Biol 428: 1912-1926).

NSF is an ATPases Associated with various cellular Activities (AAA) family protein containing three well defined domains: the N-domain, which mediates interactions with one or more α-SNAP polypeptides, the D1 ATPase domains, which couple ATP hydrolysis to force-generating conformational changes that remodel SNARE complexes, and the D2 ATPase domain, which mediates NSF hexamerization (Whiteheart et al., 2001, Int Rev Cytol 207, 71-112; Hanson and Whiteheart, 2005, Nat Rev Mol Cell Biol 6, 519-529; Zhao et al., 2010, J. Biol. Chem. 285, 761-772).

The soybean resistance-associated Rhg1 α-SNAPs comprise polymorphic variant sequences of Glyma.18G022500 that encode variant α-SNAP proteins (U.S. patent application Ser. No. 13/843,447). Rhg1 resistance-associated α-SNAPs have lower binding affinity for NSF and SNARE/NSF complexes, and disrupt vesicle trafficking in planta (Bayless et al., 2016, Proc. Natl. Acad. Sci. USA 113, E7375-E7382). The relative abundance of Rhg1-encoded defective α-SNAP variants increases substantially within host syncytium cells at the nematode feeding site (Bayless et al., 2016, Proc. Natl. Acad. Sci. USA Proc. Natl. Acad. Sci. USA 113, E7375-E7382, Proc. Natl. Acad. Sci. USA 113, E7375-E7382).

Resistance-associated Rhg1 haplotypes group into structural classes based on the type of α-SNAP polymorphisms that they encode, which also correlates with the copy-number of Rhg1 repeats that are present across hundreds of soybean accessions (Cook et al., 2014, Plant Physiol 165, 630-647; Lee et al., 2015).). Rhg1_{HC} (high copy) loci carry four or more and frequently nine or ten Rhg1 repeats, and Rhg1_{LC} (low-copy) loci carry three or fewer Rhg1 repeats. Rhg1_{LC} is also known as rhg1-a and Rhg_{HC} is also known as rhg1-b (Mitchum 2016 and Liu 2017 Nat. Commun. 8, 14822). Rhg1_{HC} and Rhg1_{LC} encode similar yet distinct α-SNAP variants that are impaired in normal α-SNAP/NSF interactions (Bayless et al., 2016, Proc. Natl. Acad. Sci. USA 113, E7375-E7382, Proc. Natl. Acad. Sci. USA 113, E7375-E7382). All Rhg1_{HC} loci examined to date also have one Rhg1 repeat that encodes a wildtype (WT) α-SNAP along with multiple repeats encoding a resistance-type α-SNAP, while Rhg1_{LC} loci encode only resistance-type α-SNAPs and no WT α-SNAP (Cook et al., 2012, Science 338, 1206-1209; Cook et al., 2014, Plant Physiol 165, 630-647; Lee et al., 2015). Plants carrying Rhg1_{HC} or Rhg1_{LC} loci exhibit elevated transcript abundance that correlates approximately with copy number for the repeat genes, including the Rhg1 α-SNAP gene, and variants thereof (U.S. Patent Application Publ. No. 2013-0305410 A1; Cook et al., 2012, Science 338, 1206-1209; Cook et al., 2014, Plant Physiol 165, 630-647).

In experiments performed in N. benthamiana leaves, high expression of these resistance-conferring α-SNAPs hindered vesicular trafficking and eventually elicited cell death, but co-expression of wild type soybean α-SNAPs diminished this cytotoxicity (Bayless et al., 2016, Proc. Natl. Acad. Sci. USA 113, E7375-E7382).

Therefore, there is a need in the art for methods and compositions that enable the generation and propagation of SCN-resistant plant cells that harbor Rhg1 resistance-associated genes, including Rhg1 resistance-associated α-SNAPs.

### SUMMARY OF THE INVENTION

The present disclosure provides methods for producing plant cells resistant to nematodes. The disclosure further provides methods for improving the growth or survival of a plant cell containing one or more Rhg1 genes capable of conferring nematode resistance. The present disclosure also provides compositions for producing plant cells resistant to nematodes, or for improving the growth or survival of a plant cell containing one or more Rhg1 genes conferring nematode resistance. In further aspects, the disclosure provides plant cells and plants with increased resistance to nematodes, without or preferably with improved growth or survival.

In some embodiments, the disclosure provides methods and compositions for producing plant cells resistant to nematodes, or for improving the growth or survival of a plant cell containing one or more Rhg1 genes capable of conferring nematode resistance, comprising increasing expression of, altering an expression pattern of, altering a polynucleotide sequence of, altering abundance or localization of a polypeptide product of, or increasing copy number of, one or more polynucleotides encoding α-SNAP proteins, or homologs or variants thereof, and/or one or more polynucleotides encoding NSF proteins, or homologs or variants thereof, wherein said plant cells are resistant to nematodes relative to native plant cells.

In certain embodiments, the disclosure provides methods of producing plant cells resistant to nematodes, or for improving the growth or survival of a plant cell containing one or more Rhg1 genes capable of conferring nematode resistance, comprising increasing expression of, altering an expression pattern of, altering a polynucleotide sequence of, altering abundance or localization of a polypeptide product of, or increasing copy number of a polynucleotide encoding one or more α-SNAP proteins with at least 95% identity to a polynucleotide identified by SEQ ID NOs: 5 or 6, or an encoded polypeptide with at least 95% identity to a polypeptide identified by SEQ ID NOs: 14 or 15, or homologs or variants thereof.

In further embodiments, the disclosure provides methods of producing plant cells resistant to nematodes, or for improving the growth or survival of a plant cell containing one or more Rhg1 genes capable of conferring nematode resistance, comprising increasing expression of, altering an expression pattern of, altering a polynucleotide sequence of, altering abundance or localization of a polypeptide product of, or increasing copy number of a polynucleotide encoding and a polynucleotide encoding one or more NSF proteins with at least 95% identity to a polynucleotide identified by SEQ ID NOS: 8 or 9, or an encoded polypeptide with at least 95% identity to a polypeptide identified by SEQ ID NOs 17 or 18, or homologs or variants thereof.

In still further embodiments, the disclosure provides methods of producing plant cells resistant to nematodes, or for improving the growth or survival of a plant cell containing one or more Rhg1 genes capable of conferring nematode resistance, comprising increasing expression of, altering an expression pattern of, altering a polynucleotide sequence of, altering abundance or localization of a polypeptide product of, or increasing copy number of both (a) a polynucleotide encoding one or more α-SNAP proteins encoded by a polynucleotide with at least 95% identity to SEQ ID NO: 5 or SEQ ID NO: 6, and (b) a polynucleotide encoding one or more NSF proteins encoded by a polynucleotide with at least 95% identity to SEQ ID NO: 9, or homologs or functionally conserved variants of any of the aforementioned SEQ ID NOs.

In embodiments, the methods of the disclosure produce plant cells or plants resistant to nematodes. In certain embodiments, the plant cells or plants provided herein are soybean, sugar beets, potatoes, corn, wheat, pea or beans or those plants listed in Tables 6 and 7.

In embodiments, the methods of the disclosure comprise increasing expression of, altering an expression pattern of, altering a polynucleotide sequence of, altering abundance or localization of a polypeptide product of, or increasing copy number of a polynucleotide cells in the root of the plant. In some embodiments, the one or more polynucleotides encoding α-SNAP proteins or NSF proteins, or homologs or variants thereof, is increased by incorporation of a construct comprising a promoter operably linked to one or more of said polynucleotides in the plant cells. In embodiments, the disclosure provides a method of increasing nematode resistance in a plant, wherein at least two of the polynucleotides recited herein have increased expression, an altered expression pattern, or increased copy number.

In one aspect, the disclosure provides a method of altering the abundance of one or more α-SNAP proteins in a plant cell. In certain embodiments of the disclosed methods, an amount of an α-SNAP encoded by the sequence identified in SEQ ID NO: 2, or a polynucleotide with at least 95% identity thereof, is reduced relative to an amount of an α-SNAP encoded by either of the sequences identified in SEQ ID NO: 5 and SEQ ID NO: 6, or polynucleotides with at least 95% 75% identity, or homologs or functionally conserved variants of the SEQ ID NO: 2, SEQ ID NO: 5, or SEQ ID NO: 6.

In a further aspect, this disclosure provides compositions for producing plant cells resistant to nematodes, or for improving the growth or survival of a plant cell containing one or more Rhg1 genes capable of conferring nematode resistance. In some embodiments, the disclosure provides constructs comprising a promoter operably linked to one or more polynucleotides encoding α-SNAP proteins, one or more polynucleotides encoding NSF proteins, or homologs or variants thereof. In further embodiments, the disclosure provides a construct comprising a polynucleotide with at least 95% identity to SEQ ID NO: 5 or SEQ ID NO: 6, and/or a polynucleotide with at least 95% identity to SEQ ID NO: 9, or homologs or functionally conserved variants of the SEQ ID NOs identified herein. In certain embodiments, a construct of the disclosure comprises a plant promoter.

In still another aspect, the disclosure provides a nematode resistant transgenic plant cell, or a transgenic plant cell containing one or more Rhg1 genes capable of conferring nematode resistance comprising with improved growth or survival. In embodiments, a transgenic plant cell of the disclosure comprises one or more polynucleotides encoding α-SNAP proteins, or one or more polynucleotides encoding NSF proteins, or homologs or variants thereof. In certain embodiments, a transgenic plant or plant cells of the disclosure comprises one or more α-SNAP proteins encoded by polynucleotides with at least 95% identity to the polynucleotides identified by SEQ ID NOS: 1-7, or polypeptides with at least 95% identity to polypeptides identified by SEQ ID NOs 10-16, or homologs or variants thereof. In further embodiments, a transgenic plant cell of the disclosure comprises one or more NSF proteins encoded by polynucleotides with at least 95% identity to the polynucleotides identified by SEQ ID NOS: 8 and 9, or comprise polypeptides with at least 95% identity to polypeptides identified by SEQ ID NOs 17 and 18, or homologs or variants thereof.

Embodiments of the disclosure also provide seeds comprising the transgenic plant cells described herein, plants grown from the seeds described herein, parts, progeny or asexual propagates of the transgenic plant cells disclosed herein. In some embodiments, the transgenic plant, plant cell or seed, or part, progeny or asexual propagate thereof of the disclosure are soybeans, sugar beets, potatoes, corn, wheat, peas or beans, or a wide variety of plant species as listed in Tables 6 and 7.

### BRIEF DESCRIPTION OF THE DRAWINGS

The following detailed description can be best understood when read in conjunction with the following drawings in which:

FIG. 1A shows an immunoblot of wild-type α-SNAPs, Rhg1 resistance-type α-SNAPs and NSF in HG type test soybean roots. Rhg1_{LC} varieties: PI 548402 (Peking), PI 89772, PI 437654, PI 90763; Rhg1_{HC} varieties: PI 88788, PI 209332, PI 548316 (7 copy). PonceauS staining shows total protein loaded per lane. FIG. 1B illustrates densitometry indicating total NSF expression in HG type test lines. FIG. 1C, shows immunoblots from trifoliate leaves or roots of Williams 82 (Wm82) and modern Rhg1_{LC} and Rhg1_{HC} varieties Forrest and Fayette (labeling as described for FIG. 1A). FIG. 1D shows immunoblots for total WT α-SNAPs and α-SNAP_{Rhg1}LC in “Forrest” (Rhg1_{LC}) transgenic roots transformed with an empty vector (EV) or the native Williams 82 α-SNAP_{Rhg1}WT locus, or in Williams 82 roots transformed with empty vector.

FIG. 2A is an alignment of soybean NSF_{Ch07}, NSF_{Ch13}, and NSF_{RAN07} N-terminal domains (SEQ ID NOs:20, 22, and 21, respectively). Large identical regions are omitted. N-domain residues that bind α-SNAP are shaded dark grey (N₂₁, RR₈₂₋₈₃, KK₁₁₇₋₁₁₈). NSF_{RAN07} polymorphisms R₄Q, N₂₁Y, S₂₅N, ₁₁₆F, M₁₈₁I are shaded light grey. FIG. 2B shows NSF_{RAN07} modeled to NSF_{CHO} cryo-EM structure (3J97A, State II). NSF residue patches implicated in α-SNAP binding are labeled I, II or III, respectively. FIG. 2C shows NSF_{RAN07} polymorphisms (N₂₁Y), with zoomed in view of polymorphic N-domain region. FIG. 2D shows that NSF N-domain R₄ is conserved in most model eukaryotes. Frequency logo of first 10 NSF N-domain residues of the following organisms: Homo sapiens, Bos taurus, Mus musculus, Cricetulus griseus (Chinese hamster), Caenorhabditis elegans, Drosophila melanogaster, Danio rerio, Xenopus laevis, Gallus gallus, Neurospora crassa, Saccharomyces cerevisiae, Schizosaccharyomyces pombe, Chlamydomonas reinhardtii, Physcomitrella patens, Zea mays, Oryza sativa, Solanum tuberosum, Cucumis sativa, Arabidopsis thaliana, Medicago truncatula, Nicotiana benthamiana, and Glycine max.

FIG. 3A is a ribbon diagram showing cryo-EM structure of mammalian 20S supercomplex, masked to show only SNARE bundle (right, “SNARE complex”), one α-SNAP (middle, “α-SNAP”) and two NSF N-domains (left and middle behind, “NSF N-Domain”). Conserved NSF N-domain patches (I, R10; II, RK67-68; III, KK104-105) and α-SNAP C-terminal contacts (D217DEED290-293) are shown extending from the ribbon depiction (see also, FIG. 3B). FIG. 3B is a ribbon diagram showing NSF_{RAN07} polymorphisms; RAN07 residues are labeled (shown black), and arrows point out the α-SNAP interacting residues (light grey). FIG. 3C is a photograph of silver-stained SDS/PAGE of recombinant NSF_{Ch07} or NSF_{RAN07} bound in vitro by the recombinant proteins indicated on second line: no-α-SNAP control (No) or wild-type (WT), low-copy (LC), or high copy (HC) Rhg1 α-SNAP. BSA: bovine serum albumin. FIG. 3D shows densitometric quantification of NSF_{Ch07} or NSF_{RAN07} bound by Rhg1 α-SNAPs in FIG. 3C; data are from three independent experiments and error bars show SEM.

FIG. 4A is a photograph of N. benthamiana leaves ˜6 days post agro-infiltration with 9:1 or 14:1 mixed cultures of α-SNAP_{Rhg1} LC and NSF_{Ch07} or NSF_{Ch13} or NSF_{RAN07} or empty vector (nine or fourteen parts Agrobacterium tumefaciens that delivers α-SNAP_{Rhg1}LC to one part Agrobacterium that delivers soybean NSF or empty vector control). FIG. 4B, same as in FIG. 4A, but 7:1 or 11:1 mixed cultures of α-SNAP_{Rhg1} LC co-expressed with NSF_{N.benth} or NSF_{Ch13} or NSF_{RAN07} or empty vector. FIG. 4C is a photograph of silver-stained SDS/PAGE of recombinant NSF_{N.benth} bound in vitro by recombinant wild-type, low-copy (LC), or high copy (HC) Rhg1 α-SNAP proteins or WT α-SNAP lacking the final 10 C-terminal residues (α-SNAP1-279). BSA, bovine serum albumin. FIG. 4D, same as in FIG. 4A and FIG. 4B, but 4:1 or 9:1 mixed cultures of α-SNAP_{Rhg1}LC or α-SNAP_{Rhg1}LC-1289A co-expressed with NSF_{Ch07} or NSF_{RAN07}.

FIG. 5A shows frequency of SoySNP50K SNP ss715597431 (corresponding to NSF_{RAN07} R₄Q) in all 19,645 SoySNP50K-genotyped Glycine max accessions. FIG. 5B shows frequency of ss715597431 in all USDA G. max with Rhg1_{LC} or Rhg1_{HC} haplotype signatures or in remainder of SoySNP50K-genotyped G. max from USDA collection. FIG. 5C and FIG. 5D show SNP mapping of the NSF_{RAN07} candidate gene interval for low copy Rhg1 and high copy Rhg1 respectively, indicating relative SNP frequencies. HG type and SoyNAM populations used for SNP mapping.

FIG. 6A is an anti-HA immunoblot of N. benthamiana leaves agroinfiltrated to express empty vector, N-HA-α-SNAP_{Ch11} or N-HA-α-SNAP_{Ch11}-IR (intron-retention). PonceauS staining indicates relative total protein levels. FIG. 6B illustrates modeling of α-SNAP_{Ch11}-IR to sec17 crystal structure (yeast α-SNAP, PDB ID 1QQE) suggests early termination of alpha-helix 12. FIG. 6C shows immunoblots for total WT α-SNAP and α-SNAP_{Rhg1}LC levels in Forrest (Rhg1_{LC}) transgenic roots transformed with an empty vector (EV) or the native WT α-SNAP_{Ch11} locus from Williams 82. FIG. 6D, as described in FIG. 5A, except frequency of SoySNP50K SNP ss715610416 allele that is closest marker for α-SNAP_{Ch11}-IR, in all 19,645 USDA accessions. FIG. 6E illustrates the frequency of ss715610416 in all USDA Glycine max with Rhg1_{LC} or Rhg1_{HC} haplotype signatures vs. remainder of SoySNP50K-genotyped USDA collection.

FIG. 7A shows immunoblot of wild-type α-SNAPs and NSF expression in HG type test soybean roots. Rhg1_{LC} varieties: PI 548402 (Peking), PI 89772, PI 437654, PI 90763; Rhg1_{HC} varieties: PI 88788, PI 209332, PI 548316 (7 copy). PonceauS staining shows total protein loaded per lane. FIG. 7B shows densitometry data on the ratio of WT α-SNAPs to Rhg1 resistance type α-SNAPs. Ratios calculated using Image J densitometry as in FIG. 1B. FIG. 7C is an agarose gel showing PCR amplicons generated with RAN07 or NSF Ch₀₇WT specific primers on HG type soybeans and soybean genome reference variety Williams82 (Wm82). Rhg1_{LC} varieties: “Forrest” (PI 548402-derived), PI 89772, PI 437654, PI 90763; Rhg1_{HC} varieties: PI 88788, PI 209332, PI 548316 (7 copy).

FIG. 8A and FIG. 8B show NSF_{RAN07} (SEQ ID NO:18) amino acid alignment with NSF_{Ch07} of soybean reference genome Williams82 (SEQ ID NO:17). N-domain amino acid polymorphisms unique to RAN07 are indicated by boldface in the corresponding residues in Wm82 NSFCh07.

FIG. 9A shows NSF_{RAN07} modeled to an NSF_{CHO} cryo-EM structure (as described in FIG. 2A), but rotated 90° on the X-axis. NSF residue patches implicated in α-SNAP binding are indicated. FIG. 9B shows that NSF N-domain R₄ is conserved in most model eukaryotes. Frequency logo of first 10 NSF N-domain residues of the following organisms: Homo sapiens, Bos taurus, Mus musculus, Cricetulus griseus (Chinese hamster), Caenorhabditis elegans, Drosophila melanogaster, Danio rerio, Xenopus laevis, Gallus gallus, Neurospora crassa, Saccharomyces cerevisiae, Schizosaccharyomyces pombe, Chlamydomonas reinhardtii, Physcomitrella patens, Zea mays, Oryza sativa, Solanum tuberosum, Cucumis sativa, Arabidopsis thaliana, Medicago truncatula, Nicotiana benthamiana, and Glycine max. FIG. 9C is an alignment of NSF N-domain using available plant NSF amino acid sequences from Phytozome.org (SEQ ID NOs:23-52). The alignment was generated with Jalview starting at a conserved methionine residue corresponding to RAN07 met 17. Residues polymorphic in RAN07 are outlined with a box with the corresponding position labeled above.

FIG. 10A shows cryo-EM structure of mammalian 20S supercomplex showing SNARE bundle similar to that of FIG. 4A. FIG. 10B depicts that same as FIG. 10A but rotated 90° on Y-axis. FIG. 10C is the same as FIG. 3C, except the recombinant NSF_{Ch07} or NSF_{RAN07} is bound in vitro by no-α-SNAP control (No) or wild-type (WT), low-copy (LC), or high copy (HC) Rhg1 α-SNAP, or WT α-SNAP truncated at final 10 residues (WT1-279). BSA: bovine serum albumin.

FIG. 11A shows N. benthamiana leaves −6 days post agro-infiltration with 1:4 or 4:1 mixed cultures of α-SNAP_{Rhg1}LC and NSF_{Ch07} or NSF_{RAN07} or α-SNAP_{Rhg1}WT or empty vector (one or three parts Agrobacterium that delivers α-SNAP_{Rhg1}LC to one part Agrobacterium that delivers soybean NSF, or α-SNAP_{Rhg1WT} or empty vector control) as in FIG. 4A. FIG. 11B shows N. benthamiana leaves like those shown in FIG. 4A, but with a 9:1 or 19:1 mixed culture of α-SNAP_{Rhg1}LC co-expressed with NSF_{Ch07} or NSF_{RAN07} or empty vector. FIG. 11C shows N. benthamiana leaves as shown in FIG. 4A, but using α-SNAP_{Rhg1HC} instead of α-SNAP_{Rhg1}LC in the corresponding mixture cultures of NSF_{Ch07} or NSF_{RAN07} or empty vector.

FIG. 11D depicts N. benthamiana leaves −6 days post agro-infiltration with 1:9 mixed cultures of NSF_{Ch07} or NSF_{RAN07} or NSF_{Ch13} or NSF_{Nbenth} to empty vector (9 parts empty vector cultures to 1part NSF expressing Agrobacterium culture). FIG. 11E shows N. benthamiana leaves similar to those shown in FIG. 4A, but with a 11:1 mixed culture of α-SNAP_{Rhg1LC} or α-SNAPRhg1Lc1-2soα-SNAP_{Rhg1LC1-280} (lacks the final 10 C-terminal residues) co-expressed with NSF_{Ch07} or NSF_{RAN07} or empty vector.

FIG. 12A and FIG. 12B show an amino acid alignment with NSF N. benthamiana (SEQ ID NO:53) and NSF_{Ch07} (SEQ ID NO:18) of soybean reference genome Williams82. NSF N-domain residues are conserved in α-SNAP binding and are shown in boldface.

FIG. 13A (SEQ ID NOs:54-88) and FIG. 13B (SEQ ID NOs:89-123) show an alignment of NSF N-domain starting from position 1 and depicts general conservation of R4. The alignment was generated with Jalview and includes all reliable Angiosperm NSF sequences available from Phytozome.org.

FIG. 14 is an immunoblot showing expression results for α-SNAP_{Rhg1}LC in independent soybean lines transformed with genes encoding α-SNAP_{Rhg1}LC and either wild-type NSF_{Ch07} or NSF_{RAN07}. Only one transformed plant was obtained for the α-SNAP_{Rhg1}LC+wild-type NSF_{Ch07} DNA construct and that plant did not actually express α-SNAP_{Rhg1}LC protein.

### DETAILED DESCRIPTION

All publications, patents and patent applications cited herein are hereby expressly incorporated by reference for all purposes.

Before describing the disclosed methods and compositions in detail, a number of terms will be defined. As used herein, the singular forms “a”, “an”, and “the” include plural referents unless the context clearly dictates otherwise.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of this invention.

For the purposes of describing and defining this invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

In addition to the methods that are more specifically described herein and/or described by reference to literature citations, methods well known to those skilled in the art (e.g., Ausubel, F., et al. (Eds.), Current Protocols in Molecular Biology, 2017; Acquaah, G. (Ed.), Principles of Plant Genetics and Breeding, 2^{nd} Edition 2012) can be used to carry out many of the manipulations disclosed herein.

As used herein, a “plant” includes any portion of the plant, including but not limited to, a whole plant, a portion of a plant such as a part of a root, leaf, stem, seed, pod, flower, cell, tissue or plant germplasm or any progeny thereof.

As used herein, soybean refers to whole soybean plant or portions thereof including, but not limited to, soybean plant cells, soybean plant protoplasts, soybean plant tissue culture cells or calli.

As used herein, a plant cell refers to cells harvested or derived from any portion of the plant or plant tissue, germplasm, cultured cells or calli.

As used herein “substantially equivalent” in terms of amino acid modification is intended to mean an amino acid that imparts, confers, or results in the substantially same function as the substituted amino acid.

As used herein, “germplasm” refers to genetic material from an individual or group of individuals or a clone derived from a line, cultivar, variety or culture, and the cells or tissues containing said genetic material. In the plural sense, “germ plasm” refers to collections of multiple lines, cultivars, varieties or cultures.

As used herein, “native polynucleotide” or “native polypeptide” refer to an endogenous polynucleotide or polypeptide in a naturally occurring chromosomal context. In contrast, an “exogenous” or “ectopic” polynucleotide or polypeptide refers to expression of a transgenic gene, or expression controlled by a non-native chromosomal context (e.g., by introduction of non-native promoters or enhancer elements).

As used herein, “nematode” is intended to mean any roundworm or unsegmented worm belonging to the phylum Nematoda

As used herein, “enhanced resistance” is intended to mean increased resistance to nematodes compared to native plants of the same species.

As used herein, “altering the expression pattern of” a gene or polypeptide comprises increasing its expression, decreasing its expression, or altering the location of its expression. As used herein, increasing, decreasing, or altering expression of a gene or polypeptide can be at the nucleotide or polypeptide level, and can comprise alterations in native or exogenous polynucleotide or polypeptide. Altering the location of expression of a gene product or polypeptide means altering the location or relative abundance in different parts of a plant. Alternatively, in some embodiments described herein, altering the location of expression means altering the sub-cellular localization of expression in a cell.

As used herein, “modification” as it refers to an amino acid, polypeptide and/or nucleotide is intended mean for example missense mutation, nonsense mutation, insertion, deletion, duplication, frameshift mutation and repeat expansion.

The Rhg1 locus is a chromosomal region identified as a region important for resistance to SCN. When used in reference to a protein, the term Rhg1 typically is not italicized, and refers to the protein products of one or more genes that are located at the Rhg1 locus. As used herein, a locus is a chromosomal region where one or more trait determinants, genes, polymorphic nucleic acids, or markers are located. A quantitative trait locus (QTL) refers to a polymorphic genetic locus where one or more underlying genes controls a trait that is quantitatively measured and contains at least two alleles that differentially affect expression of a phenotype or genotype in at least one genetic background, with said locus accounting for part but not all the observed variation in the overall phenotypic trait that is being assessed. A genetic marker is a nucleotide sequence or amino acid sequence that can be used to identify a genetically linked locus, such as a QTL. Examples of genetic markers include, but are not limited to, single nucleotide polymorphisms (SNP), simple sequence repeats (SSR; or microsatellite), a restriction enzyme recognition site change, genomic copy number of specific genes or target sequences or other sequence-based differences between a susceptible and resistant plant.

A “linked” genetic locus describes a situation in which a genetic marker and a trait are closely linked chromosomally such that the genetic marker and the trait do not independently segregate and recombination between the genetic marker and the trait does not occur during meiosis with a readily detectable frequency. The genetic marker and the trait can segregate independently, but generally do not. For example, a genetic marker for a trait can only segregate independently from the trait 5% of the time; suitably only 5%, 4%, 3%, 2%, 1%, 0.75%, 0.5%, 0.25%, or less of the time. Genetic markers with closer linkage to the trait-producing locus will serve as better markers because they segregate independently from the trait less often because the genetic marker is more closely linked to the trait. Genetic markers that directly detect polymorphic nucleotide sites that cause variation in the trait of interest are particularly useful for their accuracy in marker-assisted plant breeding. Thus, the methods of screening provided herein can be used in traditional breeding, recombinant biology or transgenic breeding programs or any hybrid thereof to select or screen for resistant varieties.

A linked locus can also describe two loci that do not reside close to each other on a chromosome, and therefore are not physically linked, but exhibit lack of independent segregation (i.e. they co-segregate). In the formal genetic sense, such a pair of co-segregating loci exhibit genetic linkage. As used herein, the terms “linked locus” and “co-segregating locus” are used interchangeably, and thus refer to physical linkage (on the same chromosome) or genetic linkage (either on the same chromosome or co-segregating on different chromosomes). A gene or locus is “associated” with another gene or locus when they are linked or co-segregate with one another. For example, a gene, allele, or locus is “associated” with Rhg1 if it co-segregates or is physically linked to the Rhg1 locus.

As used herein, Glyma.18G022700, Glyma.18G022500, Glyma.18G022400, and/or Glyma.07G195900 refer to the soybean genomic nomenclature describing those genes, the proteins or polypeptides they encode, and include any polynucleotide or polypeptide variants, naturally occurring or otherwise, and any homologues or conserved portions in other plant species. In some embodiments, Glyma.18G022700, Glyma.18G022500, Glyma.18G022400, and/or Glyma.07G195900 refer to the genes or polypeptides, and any polynucleotide or polypeptide variants, naturally occurring or otherwise, in plants of the genus Glycine, and encompass any homologues or conserved portions in other plant species. The 13-character gene names are from the Wm82.a1 genome assembly and Glyma 1.0 gene models (Schmutz et al., 2010) and the more recent 15-character gene names are from the U.S. Department of Energy Joint Genome Institute Wm82.a2 soybean genome assembly and Glyma 2.0 gene model naming revision.

The present disclosure provides methods and compositions for increasing resistance of a plant or plant cells to cyst nematodes. In some embodiments, the disclosure provides methods and compositions for generating transgenic plant materials, including transgenic cells and plants. In additional embodiments, the disclosure provides compositions comprising nucleotide constructs useful for generating transgenic cells and plants resistant to nematodes. In still further embodiments, the disclosure provides nucleotide constructs encoding Rhg1 resistance-type polypeptides, or homologs or variants thereof. In certain embodiments, Rhg1 resistance-type α-SNAPs are provided. In further embodiments, the disclosure provides Rhg1 resistance-type α-SNAPs encoded by SEQ ID NO: 5 or SEQ ID NO: 6, or homologs or variants thereof.

In some embodiments, the disclosure provides alleles associated with the Rhg1 locus due to lack of independent segregation from the locus. In certain embodiments, the disclosure provides alleles that co-segregate with Rhg1 genes despite residing on a different chromosome (i.e., despite lack of physical linkage on the same chromosome). In one aspect, alleles associated with the Rhg1 locus comprise genes that improve the growth, reproduction and/or SCN resistance of plant cells, plants, or germplasm, that carry Rhg1 SCN resistance-conferring alleles. In certain embodiments, the disclosure provides alleles of an NSF gene, wherein the alleles of an NSF gene are associated with Rhg1. In some embodiments, the disclosure provides alleles of an NSF gene, wherein the alleles of an NSF gene are associated with improved growth, or completion of the life cycle, of plants that carry SCN resistance-conferring alleles of the Rhg1 locus. In particular embodiments, the NSF gene of the disclosure is Glyma.07G195900, or variants thereof. In an exemplary embodiment, the disclosure provides alleles of NSF associated with Rhg1 encoded by SEQ ID NO: 8, a protein corresponding to SEQ ID NO: 17, or homologs or variants thereof. In other exemplary embodiments, the disclosure provides alleles of NSF encoded by SEQ ID NO: 9, a protein corresponding to SEQ ID NO: 18, or homologs or variants thereof.

Also provided are Rhg1 genes that contribute to SCN resistance (SEQ ID NOS: 1-7) and the proteins they encode (SEQ ID NOs 10-16) located within a tandem repeat present in the genomes of soybeans exhibiting resistance to cyst nematodes, including, but not limited to, P188788, Peking, Hartwig, Fayette, and Forrest. Embodiments of the Rhg1 genes that contribute to SCN resistance of the present disclosure are as described in U.S. patent application Ser. No. 13/843,447, and also as described in Cook, D. E., et al. 2012, Science 338:1206-1209, and the associated Supporting Online Material, which are incorporated herein by reference in their entirety.

In certain embodiments, the Rhg1 genes that contribute to SCN are located on a tandemly repeated segment of chromosome 18 in resistant soybeans, and silencing of one or more of three genes in the segment leads to increased susceptibility to SCN in an otherwise resistant variety. In certain embodiments, the tandemly repeated segment comprises four genes, along with part of a fifth gene, and other DNA sequences in a chromosome segment that in some described soybean accessions (Cook et al., 2012, Science 338, 1206-1209) is approximately 31 kb in length. The tandemly repeated Rhg1 chromosome segment is found in at least two copies in the SCN-resistant varieties that have been characterized to have SCN resistance due in part to the Rhg1 locus. Various resistant varieties carry three, seven or ten copies, or other numbers of copies. In the published examples the higher copy number versions of Rhg1 express higher levels of transcripts for the three genes. Higher copy number versions of Rhg1 also confer more resistance to SCN on their own (exhibit less reliance on the simultaneous presence of desirable alleles of other SCN resistance QTL such as Rhg4 in order to effectively confer resistance to HG Type 0 SCN populations), relative to Rhg1 haplotypes with lower Rhg1 repeat copy numbers.

In certain aspects, the disclosure provides transgenic plants or transgenic plant cells with increased resistance to cyst nematodes, particularly SCN, carrying one or a plurality of transgenes encoding a non-native or exogenous Rhg1 derived, or Rhg1 associated, polynucleotide encoding one or more of the polynucleotides of SEQ ID NOs:1-9 or the polypeptides of SEQ ID NOs:10-18. Non-transgenic plants carrying these polypeptides, or bred or otherwise engineered to express increased levels of these polypeptides or the polynucleotides encoding these polypeptides, are also provided.

In some aspects, the disclosure provides methods and compositions for increasing resistance of a plant or plant cell to cyst nematodes, including but not limited to SCN, by increasing expression of, or altering an expression pattern of, or increasing copy number of one or more Rhg1 genes corresponding to the Glycine max genes designated Glyma.18G022700 (SEQ ID NO:3), Glyma.18G022500 (SEQ ID NO: 2), variants of Glyma.18G022500 (SEQ ID NO:5 or SEQ ID NO:6), and/or Glyma.18G022400 (SEQ ID NO: 1), polypeptides or functional fragments or variants thereof in cells of the plant are also provided. In another aspect, the disclosure provides methods and compositions for producing a plant or plant cell with increased resistance to cyst nematodes, including but not limited to SCN, by increasing expression of, or altering an expression pattern of, or increasing copy number of one or more Rhg1 associated genes corresponding to Glyma.07G195900 (SEQ ID NO: 8 or SEQ ID NO: 9). In embodiments, the methods and compositions of the disclosure further comprise increasing the expression of, or altering the expression pattern of, or increasing the copy number of, a polynucleotide encoding an NSF allele or a polypeptide product of said allele, in combination with one or more of the Rhg1, or Rhg1 associated, genes above. The polynucleotides of the disclosure can be 75%, 80%, 85%, 90%, 95%, 97%, 98%, 99% or 100% identical to the sequences provided.

In another aspect, the disclosure provides methods and compositions for increasing plant growth, seed production, or completion of the life cycle of plants in which resistance to SCN has been manipulated by increasing expression of, or altering an expression pattern of, or increasing copy number of Rhg1 genes. In certain embodiments, methods for increasing plant growth, seed production or completion of the life cycle of plants in which resistance to SCN has been manipulated comprise increasing expression of, altering expression pattern of, or increasing copy number of one or more polynucleotides encoding an NSF protein. In some embodiments, methods for increasing plant growth, seed production or completion of the life cycle of plants in which resistance to SCN has been manipulated comprise increasing expression of, altering an expression pattern of, or increasing copy number of a polynucleotide corresponding to Glyma.07G195900. In particular embodiments of the disclosure, a polynucleotide corresponding to Glyma.07G195900 comprises a polynucleotide identified in SEQ ID NO: 8 or SEQ ID NO: 9, polypeptides or functional fragments or variants thereof. The polynucleotide can be 75%, 80%, 85%, 90%, 95%, 97%, 98%, 99% or 100% identical to the sequences provided. In embodiments, the methods and compositions of the disclosure further comprise increasing the expression of, or altering the expression pattern of, or increasing the copy number of, a polynucleotide encoding an NSF allele or a polypeptide product of said allele, in combination with one or more of the Rhg1, or Rhg1 associated, genes above.

In still another aspect, the disclosure provides methods and compositions for increasing plant growth, seed production or completion of the life cycle of plants that contain Rhg1 alleles that contribute to SCN resistance by increasing expression of, or altering an expression pattern of, or increasing copy number of genes associated with, or linked with, Rhg1 genes that contribute to SCN resistance. In certain embodiments, the disclosure provides methods of increasing expression of, or altering an expression pattern of, or increasing copy number of a gene or protein corresponding to the Glycine max gene designated Glyma.07G195900. In still further embodiments, the disclosure provides methods and compositions for increasing plant growth, seed production, or completion of the life cycle of plants that contain Rhg1 alleles that contribute to SCN resistance, by increasing expression of, or altering an expression pattern of, or increasing copy number of one or more polynucleotides identified by SEQ ID NO: 8 or SEQ ID NO:9, a polypeptide sequence identified by SEQ ID NO: 17 or SEQ ID NO:18, or homologues, or variants thereof.

In certain embodiments, the disclosure provides transgenic plants or transgenic plant cells comprising one or more polynucleotides encoding an α-SNAP protein variant. In particular embodiments, the α-SNAP protein variant or variants confer reduced or substantially disrupted cellular vesicular trafficking in cells. In some embodiments, the α-SNAP protein variant or variants exhibit disrupted disassembly and reuse of the four-protein bundles of SNARE proteins that form when t-SNARE and v-SNARE proteins anneal during vesicle docking to target membranes.

Certain embodiments of the disclosure provide an α-SNAP protein variant corresponding to the gene designated Glyma.18G022500. In some embodiments, an α-SNAP protein variant of the disclosure corresponds to the Glyma.18G022500 from Fayette or Peking soybean lines. In particular embodiments, the α-SNAP protein variant (or variants) of the disclosure are encoded by polynucleotides identified by SEQ ID NO:5 or SEQ ID NO:6, polypeptides identified by SEQ ID NO: 14 or SEQ ID NO: 15, or functional fragments or variants thereof.

In some embodiments, the α-SNAPs of the disclosure exhibit reduced or substantially disrupted binding to wild-type NSF and to SNARE/NSF complexes. For example, in certain embodiments, the α-SNAPs of the present disclosure harbor point mutations, substitutions, deletions, or other mutagenic sequence variants. In particular embodiments, the point mutations, substitutions, deletions, or other mutagenic sequence variants of the α-SNAPs disclosed herein are localized to the C-terminus of the protein. In specific particular embodiments, the α-SNAPs of the present disclosure comprise a soybean α-SNAP sequence with one or more variant C-terminal residues in the polypeptide sequence at conserved residues Q₂₀₃, D₂₀₈, DEED₂₄₃₋₂₄₆ (SEQ ID NO:124), or EEDD₂₈₄₋₂₈₇ (SEQ ID NO:125). In other embodiments, the α-SNAPs of the present disclosure comprises one or more variant c-terminal residues in the polypeptide sequence at conserved residues in rat α-SNAP at D₂₁₇, E₂₄₉, EE₂₅₂-253, or DEED₂₉₀₋₂₉₃ (SEQ ID NO:126).

In some embodiments, the α-SNAP proteins are modified by amino acids modification at positions corresponding to positions 203, 208, 284, 285, 286, and 287 by α-SNAP numbering as set forth in SEQ ID NOS: 11, 14, or 15. Positions 203 208, 284, 285, 286, and 287 correspond to the C-terminal of the Rhg1 haplotype. In one aspect modifications present in the low copy (LC) of Glyma.18G022500 is critical to nematode resistance. The modifications D208E and expression of EEDD₂₈₄₋₂₈₇ (SEQ ID NO:125), confer enhanced resistance of the soybean against the nematode.

In another embodiment, the modified polynucleotides encode a modified α-SNAP polypeptide, wherein the modified α-SNAP polypeptide comprises: a replacement at position D286 that is D286F, or D286W, or D286Y; and a replacement at position D287 that is D287E or remains D287; and an insertion after position 287 that is (ins)288A, (ins)288G, (ins)2881, (ins)288L, (ins)288M, or (ins)288V; and a replacement at position L288 that is L288A, L288G, L2881, L2881, L288M, or L288V, or a functional equivalent amino acid to the WT amino acid expressed at position 285, 286, 287, or 288, each by α-SNAP numbering relative to the positions set for in SEQ ID NO: 11.

In yet other embodiments the encoded modified α-SNAP has one or more polynucleotides that encode a modified an α-SNAP polypeptide wherein the modified polypeptide comprises other amino acids in the same family. In one aspect D208E can be modified to any functional equivalent amino acid. In another aspect, any or both E284 and E285 can also be modified to E284D or E285D or any functionally equivalent amino acid. In yet another aspect, any or both of D286 and D287 can be also be modified to D286E or D287E or any functional equivalent amino acid. The numbering presented herein is relative to the positions in SEQ ID NO: 11. In some embodiments the encoded modified α-SNAP polypeptides comprises amino acid modifications selected from a combination of wild type amino acids or functional equivalent amino acid substitutions at positions 208, 284, 285, 286, and 287 or adjacent residues. The number presented herein is relative to the positions in SEQ ID. NO: 11.

In some embodiments, the NSF variants of the disclosure exhibit reduced or substantially disrupted binding to α-SNAP proteins. In certain embodiments, the NSF variants of the disclosure exhibit reduced or substantially disrupted binding to “wild-type” α-SNAP proteins, such as an α-SNAP protein encoded by Glyma.18G022500 haplotype of soybean accession Williams 82 (SEQ ID NO: 2), homologues, or functionally conserved variants thereof. For example, in certain embodiments, the NSF variants of the present disclosure harbor point mutations, substitutions, deletions, or other mutagenic sequence variants. In embodiments, the point mutations, substitutions, deletions, or other mutagenic sequence variants of NSF are localized to regions near the N-terminus of the protein. In particular embodiments, the NSF variants of the present disclosure comprise an NSF protein with one or more variant N-terminal residues at conserved residues corresponding to R₁₀ or RK₁₁₄₋₁₁₅ in the Chinese hamster NSF protein sequence. In some embodiments, the NSF of the present disclosure comprises a soybean NSF protein with one or both of an N₂₁Y mutation or a A_{116F} mutation in the soybean NSF protein sequence. The A_{116F} notation refers to an insertion of an additional amino acid, in this case “F” or phenylalanine, as the one hundred sixteenth amino acid of the protein.

In some embodiments, the NSF variants of the disclosure exhibit enhanced or substantially improved binding to α-SNAP proteins associated with improved plant resistance to cyst nematodes. For example, in certain embodiments, the NSF variants of the present disclosure harbor point mutations, substitutions, deletions, or other mutagenic sequence variants that facilitate binding to, or functionally interacting with, a variant α-SNAP protein that is less capable of binding to a “wild-type” NSF protein. In embodiments, the point mutations, substitutions, deletions, or other mutagenic sequence variants of NSF that facilitate binding to, or functionally interacting with, a variant α-SNAP protein that is less capable of binding to a “wild-type” NSF protein, are localized to the regions near the N-terminus of the protein. In particular embodiments, the NSF variants of the present disclosure that facilitate binding to, or functionally interacting with, a variant α-SNAP protein that is less capable of binding to a “wild-type” NSF protein comprise an NSF protein with one or more variant N-terminal residues at conserved residues corresponding to R₁₀ or RK₁₁₄₋₁₁₅ in the Chinese hamster NSF protein sequence. In some embodiments, the NSF variants of the disclosure that facilitate binding to, or functionally interacting with, a variant α-SNAP protein that is less capable of binding to a “wild-type” NSF protein comprises a soybean NSF protein with one or both of an N₂₁Y mutation or a ^{\hat{ }}₁₁₆F mutation in the soybean NSF protein sequence.

In some embodiments, the NSF proteins are modified by amino acid mutations at positions 4, 21, 25, 116, and 181 by NSF numbering as set for in SEQ ID NOS:17 or 18. The mutations enhance growth and viability of the plant versus plants that express the wild type NSF sequence as provided in SEQ ID NO: 17. The amino acid mutations at positions 4 and 21 enhance growth and viability of the plant. In some embodiments the encoded modified polypeptides comprises amino acid modifications selected from the modifications: R4N/N21F; R4N/N21W; R4N/N21Y; R4C/N21F; R4C/N21W; R4C/N21Y; R4Q/N21F; R4Q/N21W; R4Q/N21Y; R4S/N21F; R4S/N21W; R4S/N21Y; R4T/N21F; R4T/N21W; and R4T/N21Y, each with number relative to positions set forth in SEQ ID NOS: 17 or 18.

In yet another embodiment the encoded modified NSF has one or more polynucleotides alterations that encode a modified NSF protein wherein the modified polypeptide comprises other amino acids in the same family. In one aspect, R4 can be modified to amino acids N, C, Q, S or T or any functionally equivalent amino acid. In yet another aspect the amino acid at position 21 can be modified to F, W, or any functionally equivalent amino acid. In another, aspect S25 can be optionally modified to N or a functionally equivalent amino acid. In still another embodiment the optional gap at position 116 can be optionally modified to an F or functionally equivalent amino acid. In still another aspect, the M at 181 can be optional modified to an I or functionally equivalent amino acid. The numbering herein is relative to the positions in SEQ ID NO: 17.

In certain embodiments, expression of α-SNAP variants disclosed herein is substantially toxic, or lethal, or otherwise intolerable, to a plant or transgenic plant, or plant cell in which it is expressed, unless a complementary NSF protein is co-expressed. In certain embodiments, an α-SNAP protein with point mutations, substitutions, deletions, or other mutagenic sequence variants that are toxic to a transgenic plant or plant cell, is co-expressed with one or more NSF variants with point mutations, substitutions, deletions, or other mutagenic sequence variants. In particular embodiments, one or more α-SNAP proteins with C-terminal point mutations, substitutions, deletions, or other mutagenic sequence is co-expressed with one or more NSF proteins with point mutations, substitutions, deletions, or other mutagenic sequence. In embodiments, α-SNAP proteins with C-terminal point mutations, substitutions, deletions, or other mutagenic sequence is co-expressed with one or more NSF proteins with mutations localized to the regions near the N-terminus of the protein. In particular embodiments, the NSF variants of the present disclosure comprise an NSF protein with one or more variant N-terminal residues at conserved residues corresponding to R₁₀ or RK₁₁₄₋₁₁₅ in the Chinese hamster NSF protein sequence. In some embodiments, the NSF of the present disclosure comprises a soybean NSF protein with one or both of an N₂₁Y mutation or a ^{\hat{ }}₁₁₆F mutation in the soybean NSF protein sequence. In other particular embodiments, the NSF of the present disclosure comprises a soybean NSF protein as identified in SEQ ID NO: 18 or encoded by a polynucleotide as identified in SEQ ID NO: 9, or homologues or functionally conserved variants thereof.

In certain embodiments, an NSF protein is expressed in a plant or plant cell containing the Rhg1 tandem repeat segment. In exemplary embodiments, NSF protein variants are expressed in a plant or plant cell containing the Rhg1 tandem repeat segment. In certain embodiments, the NSF variants expressed in a plant or plant cell containing the Rhg1 tandem repeat segment comprise an NSF protein with one or more variant N-terminal residues at conserved residues corresponding to R₁₀ or RK₁₁₄₋₁₁₅ in the Chinese hamster NSF protein sequence. In some embodiments, the NSF variant expressed in a plant or plant cell containing the Rhg1 tandem repeat segment comprises a soybean NSF protein with one or both of an R₄Q mutation, an N₂₁Y mutation, or a ^{\hat{ }}₁₁₆F mutation in the soybean NSF protein sequence.

In various embodiments disclosed herein, an NSF protein is expressed in plants or plant cells that also carry Rhg1He (high copy) loci carrying four or more, and frequently nine or ten, Rhg1 repeats. In other embodiments, an NSF protein is expressed in plants or plant cells that also carry Rhg1_{LC} (low-copy) loci carrying three or fewer Rhg1 repeats. (Rhg1Lc is also known as rhg1-a and Rhg1He is also known as rhg1-b.) Rhg1_{HC} and Rhg1_{Lc} encode similar yet distinct α-SNAP variants that are impaired in normal α-SNAP-NSF interactions (Bayless et al., 2016, Proc. Natl. Acad. Sci. USA 113, E7375-E7382).

In further embodiments, the disclosure provides methods and compositions for producing plant cells with increased resistance to nematodes comprising reducing a level of a “wild-type” α-SNAP allele relative to a variant α-SNAP allele. In some embodiments, the level of an α-SNAP encoded by the sequence identified in SEQ ID NO: 2 is reduced relative to a variant α-SNAP encoded by either of the sequences identified in SEQ ID NO: 5 and SEQ ID NO: 6.

In alternative embodiments, a variant NSF protein capable of functionally complementing one or more variant α-SNAP genes is expressed in a plant cell that contains the one or more variant α-SNAP genes. In embodiments, the variant NSF protein capable of functionally complementing one or more variant α-SNAP genes improves the growth of a cell expressing the variant α-SNAP genes. In further embodiments, a variant NSF protein capable of functionally complementing one or more variant α-SNAP genes confers cyst nematode resistance on a cell expressing the variant α-SNAP genes. In certain embodiments, the one or more variant α-SNAP genes disclosed herein function analogously to α-SNAP alleles encoded by Rhg1_{HC} or Rhg1_{LC}, and/or α-SNAP alleles similar to Rhg1_{HC} or Rhg1_{LC} that have been generated or introduced at other loci in the soybean genome. In still further embodiments, the one or more variant α-SNAP genes disclosed herein impact α-SNAP function in a manner similar to the αSNAPs encoded by Rhg1_{HC} or Rhg1_{LC} α-SNAP alleles. In yet further embodiments, the variant α-SNAP genes disclosed herein alter expression patterns relative to the wild-type α-SNAP protein encoded at the single-copy Rhg1 locus of soybean accession Williams 82.

In a certain aspect, the methods of the disclosure provide a breeding stock of a Rhg1 plant expressing an NSF variant. Also provided are methods of breeding a Rhg1 plant expressing one or more NSF variants. In addition, methods of growing or improving the lifecycle of a Rhg1 plant expressing one or more NSF variants are provided.

In other embodiments, the amino acids at the NSF and α-SNAP binding interface can be manipulated to enhance nematode resistance of plant species. In one aspect NSF amino acid residues 4, 21, 25, 116, 181 or adjacent residues with numbering relative to the NSF polypeptide set forth in SEQ ID NOS: 17 or 18 are mutated.

In another aspect residues 208, 284, 285, 286, 287, or adjacent residues of α-SNAP are mutated to impact the NSF/α-SNAP interface. The amino acid mutations at the binding interface of NSF/α-SNAP can enhance nematode resistance versus the wild type plant.

In another aspect, amino acids residing at the NSF/α-SNAP protein interaction interface can be mutated to achieve enhanced nematode resistance and plant viability and growth. For instance, NSF amino acid residues 4, 21, 25, 116, 181 or adjacent residues with numbering relative to the NSF polypeptide set forth in SEQ ID NOS: 17 or 18 interact with α-SNAP as designated in the NSF/α-SNAP/SNARE protein structure PDB ID code 3j97. Residues 208, 284, 285, 286, and 287 of α-SNAP or other α-SNAP residues that are at, or adjacent to residue at the NSF/α-SNAP 1 protein interaction interface with numbering relative to the NSF polypeptide set forth in SEQ ID NO: 11 can also be mutated to confer nematode resistance and plant cell growth viability.

In certain embodiments, the methods of the disclosure confer resistance to cyst nematode. Resistance (or susceptibility) to cyst nematode, including but not limited to SCN, can be measured in a variety of ways, several of which are known to those of skill in the art. In some embodiments of the disclosure, soybean roots are experimentally inoculated with SCN and the ability of the nematodes to mature (molt and proceed to developmental stages beyond the J2) on the roots is evaluated as compared to a susceptible and/or resistant control plant. A SCN greenhouse test is also described in U.S. Patent Application Publ. No. 2013-0305410 A1, which is incorporated herein in its entirety, and provides an indication of the number of cysts on a plant and is reported as the female index. Increased resistance to nematodes can also be manifested as a shift in the efficacy of resistance with respect to particular nematode populations or genotypes. Additionally, but not exclusively, SCN-susceptible soybeans grown on SCN-infested fields will have significantly decreased crop yield as compared to a comparable SCN-resistant soybean. Improvement of any of these metrics has utility even if all of the above metrics are not altered.

In certain embodiments, expression of one or more of the polynucleotides and polypeptides described in SEQ ID NOS: 1-18 is increased in a root of the plant. Suitably, expression of these polynucleotides and polypeptides is increased in root cells of the plant. The plant is suitably a soybean plant or portions thereof. In particular embodiments, these polynucleotides can also be transferred into other non-soybean plants, or homologs of these polypeptides or polynucleotides encoding these polypeptides from other plants, or synthetic genes encoding products similar to the polypeptides encoded or identified by SEQ ID NOS: 1-18 can be overexpressed in those plants. Example of such other plants include but are not limited to sugar beets, potatoes, corn, wheat, peas, and beans. Overexpression of these genes can increase resistance of plants from these other species to nematodes and in particular cyst nematodes, such as the soybean cyst nematode Heterodera glycines, the sugar beet cyst nematode Heterodera schacthii, the potato cyst nematodes Globodera paflida and related nematodes that cause similar disease on potato such as Globodera rostochiensis, the cereal cyst nematode Heterodera avenae, the corn cyst nematode Heterodera zeae, and the pea cyst nematode Heterodera goettingiana.

Expression of these polynucleotides in the various embodiments disclosed herein can be increased by increasing the copy number of these polynucleotide in the plant, in cells of the plant, suitably root cells, or by identifying plants in which this has already occurred. In some embodiments, the expression of these polynucleotides in the various embodiments can be increased using recombinant DNA technology, e.g., by using strong promoters to drive increased expression of one or more polynucleotides.

In some embodiments, expression of polynucleotides or polypeptides of the disclosure is reduced relative to the native amount. Reduction of a polynucleotide amount can be accomplished according to methods known in the art, such as reducing the mRNA level of a polynucleotide by interfering with promoter or enhancer function or modifying a promotor or enhancer. Alternatively, a polynucleotide amount can be reduced post-transcriptionally, such as by using antisense, morpholino, or small-interfering RNA, or by modifying the gene encoding the polynucleotide to reduce the stability of the mRNA or reduce or eliminate its translation. In embodiments, the amount of a protein is reduced, such as by peptide directed protein knockdown (e.g., as described in US Patent App. Publ. No. US 2015-0266935 A1), or other protein knock-down techniques known to the art (see, e.g., Bonger, K. M., et al. (2001) Nature Chemical Biology 7, 531-537; Banaszynski, L. A., et. al. (2006), Cell 126, 995-1004; Neklesa, T. K. et al. (2011) Nature Chemical Biology 7, 538-543.)

Expression of Glyma.18G022700, Glyma.18G022500, Glyma.18G022400, and/or Glyma.07G195900 can be increased in a variety of ways including several apparent to those of skill in the art and can include transgenic, non-transgenic and traditional breeding methodologies. For example, expression of the polypeptide encoded by Glyma.18G022700, Glyma.18G022500, Glyma.18G022400, and/or Glyma.07G195900 cancan be increased by introducing a construct including a promoter operational in the plant operably linked to a polynucleotide encoding the polypeptide into cells of the plant. Suitably, the cells are root cells. Alternatively, the expression of the polypeptide encoded by Glyma.18G022700, Glyma.18G022500, Glyma.18G022400, and/or Glyma.07G195900 cancan be increased by introducing a transgene including a promoter operational in the plant operably linked to a polynucleotide encoding the polypeptide into cells of the plant. The promoter can be a constitutive or inducible promoter capable of inducing expression of a polynucleotide in all or part of the plant, plant roots or plant root cells. In another embodiment, expression of Glyma.18G022700, Glyma.18G022500, Glyma.18G022400, and/or Glyma.07G195900 can be increased by increasing expression of the native polypeptide in a plant or in cells of the plant, such as the plant root cells. In another embodiment, expression of Glyma.18G022700, Glyma.18G022500, Glyma.18G022400, and/or Glyma.07G195900 can be increased by increasing expression of the native polypeptide in a plant or in cells of the plant such as the nematode feeding site, the syncytium, or cells adjacent to the syncytium. In another embodiment, expression of Glyma.18G022700, Glyma.18G022500, Glyma.18G022400, and/or Glyma.07G195900 can be increased by increasing expression of the native polypeptide in a plant or in cells of the plant such as sites of nematode contact with plant cells. In another embodiment, expression can be increased by increasing the copy number of Glyma.18G022700, Glyma.18G022500, Glyma.18G022400, and/or Glyma.07G195900. Other mechanisms for increasing expression of Glyma.18G022700, Glyma.18G022500, Glyma.18G022400, and/or Glyma.07G195900 can include, but are not limited to, increasing expression of a transcriptional activator, reducing expression of a transcriptional repressor, addition of an enhancer region capable of increasing expression of Glyma.18G022700, Glyma.18G022500, Glyma.18G022400, and/or Glyma.07G195900, increasing mRNA stability, altering DNA methylation, histone acetylation or other epigenetic or chromatin modifications in the vicinity of the relevant genes, altering protein or polypeptide subcellular localization, or increasing protein or polypeptide stability.

In addition, methods of increasing resistance of a plant to cyst nematodes can be achieved by cloning sequences upstream from Glyma.18G022700, Glyma.18G022500, Glyma.18G022400, and/or Glyma.07G195900 from resistant lines into susceptible lines. For these methods, nucleotide sequences having at least 60%, 70% or 80% identity to nucleotide sequences that flank the protein-coding regions of Glyma.18G022700, Glyma.18G022500, Glyma.18G022400, and/or Glyma.07G195900 (or sequences having at least 75%, 80%, 85%, or 90% identity to those protein-coding regions), said flanking regions including 5′ and 3′ untranslated regions of the mRNA for these genes, and also including any other genomic DNA sequences that extend from the protein coding region of these genes to the protein coding regions of immediately adjacent genes can be used.

In addition to the traditional use of transgenic technology to introduce additional copies or increase expression of the genes and mediate the increased expression of the polypeptides of the disclosure in plants, transgenic or non-transgenic technology can be used in other ways to increase expression of the polypeptides. For example, plant tissue culture and regeneration, mutations or altered expression of plant genes other than those expressly recited herein, or transgenic technologies, can be used to create instability in the Rhg1 locus or the plant genome more generally that create changes in Rhg1 locus, or Rgh1 associated gene, copy number or gene expression behavior. The new copy number or gene expression behavior can then be stabilized by removal of the variation-inducing mutations or treatments, for example by further plant propagation or a conventional cross. Examples of transgenic technologies that might be used in this way include targeted zinc fingers, ribozymes or other sequence-targeted enzymes that create double stranded DNA breaks at or close to the Rhg1 locus or Rgh1 associated gene, the cre/loxP system from bacteriophage lambda, Transcription Activator-Like Effector Nucleases (TALENs), Clustered Regularly Interspaced Short Palindromic Repeat (CRISPR) systems using CRISPR-associated proteins such as Cas9 or other nucleases, artificial DNA or RNA sequences designed to recombine with Rhg1 that can be introduced transiently, or enzymes that “shuffle” DNA such as the mammalian Rag1 enzyme or DNA transposases. Mutations or altered expression of endogenous plant genes involved in DNA recombination, DNA rearrangement and/or DNA repair pathways are additional examples.

Non-transgenic means of generating soybean varieties carrying traits of interest such as increased resistance to SCN are available to those of skill in the art and include traditional breeding, chemical or other means of generating chromosome abnormalities, such as chemically induced chromosome doubling and artificial rescue of polyploids followed by chromosome loss, knocking-out DNA repair mechanisms or increasing the likelihood of recombination or gene duplication by generation of chromosomal breaks. Other means of non-transgenically increasing the expression or copy number include the following: screening for mutations in plant DNA encoding miRNAs or other small RNAs, plant transcription factors, or other genetic elements that impact Glyma.18G022700, Glyma.18G022500, Glyma.18G022400, and/or Glyma.07G195900 expression; screening large field or breeding populations for spontaneous variation in copy number or sequence at Rhg1 or Glyma.07G195900 by screening of plants for nematode resistance, Rhg1 copy number or other Rhg1 or Glyma.07G195900 gene or protein expression traits as described in preceding paragraphs; crossing of lines that contain different or the same copy number at Rhg1 or Glyma.07G195900 but have distinct polymorphisms on either side, followed by selection of recombinants at Rhg1 or Glyma.07G195900 using molecular markers from two distinct genotypes flanking the Rhg1 or Glyma.07G195900 locus; chemical or radiation mutagenesis or plant tissue culture/regeneration that creates chromosome instability or gene expression changes, followed by screening of plants for nematode resistance, Rhg1 or Glyma.07G195900 copy number or other Rhg1 or Glyma.07G195900 gene or protein expression traits as described in preceding paragraphs; or introduction by conventional genetic crossing of non-transgenic loci that create or increase genome instability into Rhg1- or Glyma.07G195900-containing lines, followed by screening of plants for either nematode resistance or Rhg1 copy number. Examples of loci that could be used to create genomic instability include active transposons (natural or artificially introduced from other species), loci that activate endogenous transposons (for example mutations affecting DNA methylation or small RNA processing such as equivalent mutations to met1 in Arabidopsis or mop1 in maize), mutation of plant genes that impact DNA repair or suppress illegitimate recombination such as those orthologous or similar in function to the Sgs1 helicase of yeast or RecQ of E. coli, or overexpression of genes such as RAD50 or RAD52 of yeast that mediate illegitimate recombination. Those of skill in the art can find other transgenic and non-transgenic methods of increasing expression of Glyma.18G022700, Glyma.18G022500, Glyma.18G022400, and/or Glyma.07G195900.

Polynucleotides and/or polypeptides described and used herein can encode the full-length or a functional fragment of Glyma.18G022700, Glyma.18G022500, and/or Glyma.18G022400, from the Rhg1 locus, or Glyma.07G195900, or a naturally occurring or engineered variant of Glyma.18G022700, Glyma.18G022500, Glyma.18G022400, and/or Glyma.07G195900, or a derived polynucleotide or polypeptide all or part of which is based upon nucleotide or amino acid combinations similar to all or portions of Glyma.18G022700, Glyma.18G022500, Glyma.18G022400, and/or Glyma.07G195900 or their encoded products. Additional polynucleotides encoding polypeptides can also be included in the construct such as Glyma18g02600 (which encodes the polypeptide of SEQ ID NO:4). The polypeptide can be at least 75% 80%, 85%, 90%, 95%, 97%, 98%, 99% or 100% identical to the sequences provided herein. The polynucleotides encoding the polypeptides can be at least 50%, 60%, 65%, 70%, 75%, 80%, 85%, 90%, 95% or 100% identical to the sequences available in the public soybean genetic sequence database.

Expression of the polypeptide encoded by Glyma.18G022700, Glyma.18G022500, Glyma.18G022400, and/or Glyma.07G195900 can be increased, suitably the level of polypeptide is increased at least 1.2, 1.5, 1.7, 2, 3, 4, 5, 7, 10, 15, 20 or 25-fold in comparison to the untreated, susceptible or other control plants or plant cells. Control cells or control plants are comparable plants or cells in which Glyma.18G022700, Glyma.18G022500, Glyma.18G022400, and/or Glyma.07G195900 expression has not been increased, such as a plant of the same genotype transfected with empty vector or transgenic for a distinct polynucleotide.

The increase in expression of Glyma.18G022700, Glyma.18G022500, Glyma.18G022400, and/or Glyma.07G195900 in the plant can be measured at the level of expression of the mRNA or at the level of expression of the polypeptide encoded by Glyma.18G022700, Glyma.18G022500, Glyma.18G022400, and/or Glyma.07G195900. The level of expression can be increased relative to the level of expression in a control plant as shown in the Examples. The control plant can be an SCN-susceptible plant or an SCN-resistant plant. For example, a susceptible plant such as ‘Williams 82’ can be transformed with an expression vector such that the roots of the transformed plants express increased levels of Glyma.18G022700, Glyma.18G022500, Glyma.18G022400, and/or Glyma.07G195900 as compared to an untransformed plant or a plant transformed with a construct that does not change expression of Glyma.18G022700, Glyma.18G022500, Glyma.18G022400, and/or Glyma.07G195900, resulting in increased resistance to nematodes. Alternatively, the control can be a plant partially resistant to nematodes and increased expression of Glyma.18G022700, Glyma.18G022500, Glyma.18G022400, and/or Glyma.07G195900 can result in increased resistance to nematodes. Alternatively, the plant can be resistant to nematodes and increasing expression of Glyma.18G022700, Glyma.18G022500, Glyma.18G022400, and/or Glyma.07G195900 can result in further increased resistance to nematodes. Alternatively, the plant can be more resistant to certain nematode populations, races, Hg types or strains and less resistant to other nematode populations, races, Hg types or strains, and increasing expression of Glyma.18G022700, Glyma.18G022500, Glyma.18G022400, and/or Glyma.07G195900 can result in increased resistance to certain of these nematode populations, races, Hg types or strains.

Increased resistance to nematodes can be measured as described above. Increased resistance in a transgenic cell of the disclosure can be measured relative to a “native” cell not having any introduced polynucleotide sequences, or exogenous polynucleotide or polypeptide control elements. Increased resistance can be measured by the plant having a lower percentage of invading nematodes that develop past the J2 stage, a lower rate of cyst formation on the roots, reduced SCN egg production within cysts, reduced overall SCN egg production per plant, and/or greater grain yield of SCN-infested soybeans on a per-plant basis or a per-growing-area basis as compared to a control plant grown in a similar growth environment. Other methods of measuring SCN resistance also will be known to those with skill in the art. In methods of increasing resistance to nematodes described herein, the resulting plant can have at least 10% increased resistance as compared to the untreated or control plant or plant cells. Suitably the increase in resistance is at least 15%, 20%, 30%, 50%, 100%, 200%, 500% as compared to a control. Suitably, the female index of the plant with increased resistance to nematodes is about 80% or less of the female index of an untreated or control plant derived from the same or a similar plant genotype, infested with a similar nematode population within the same experiment. More suitably, the female index after experimental infection is no more than 60%, 40%, or 20% of that of the control plant derived from the same or a similar plant genotype, infested with a similar nematode population within the same experiment. Suitably, when grown in fields heavily infested with SCN (for example, more than 2500 SCN eggs per 100 cubic centimeters of soil), soybean grain yields of field-grown plants are 2% greater than isogenic control plants. More suitably, the grain yield increase is at least 3%, 4%, or 5% over that of isogenic control plants grown in similar environments.

Also provided herein are constructs including a promoter operably linked to one or more of a Glyma.18G022700, Glyma.18G022500, Glyma.18G022400, and/or Glyma.07G195900 polynucleotide encoding a polypeptide comprising SEQ ID NO: 12, SEQ ID NO: 11, SEQ ID NO: 15, SEQ ID NO: 16, SEQ ID NO: 10, SEQ ID NO: 18, or a fragment or variant thereof. Also included are homologs or variants of these sequences from other soybean varieties. The constructs can further include other genes. The constructs can be introduced into plants to make transgenic plants or can be introduced into plants, or portions of plants, such as plant tissue, plant calli, plant roots or plant cells. Suitably the promoter is a plant promoter, suitably the promoter is operational in root cells of the plant. The promoter can be tissue specific, inducible, constitutive, or developmentally regulated. The constructs can be an expression vector. Constructs can be used to generate transgenic plants or transgenic cells. The polypeptide can be at least 75%, 80%, 85%, 90%, 95%, 97%, 98%, 99% or 100% identical to the sequences of SEQ ID NO: 12, SEQ ID NO: 11, SEQ ID NO: 15, SEQ ID NO: 16, SEQ ID NO: 10, or SEQ ID NO: 18. The constructs can comprise all three polynucleotides and can mediate expression of all three polypeptides.

Transgenic plants including a non-native or exogenous polynucleotide encoding the rhg1-b polypeptides identified and described herein are also provided. Suitably these transgenic plants are soybeans. The transgenic plants express increased levels of Glyma.18G022700, Glyma.18G022500, Glyma.18G022400, and/or Glyma.07G195900 polypeptide as compared to a control non-transgenic plant from the same line, variety or cultivar or a transgenic control expressing a polypeptide other than Glyma.18G022700, Glyma.18G022500, Glyma.18G022400, and/or Glyma.07G195900. These transgenic plants also have increased resistance to nematodes, in particular SCN, as compared to a control plant. Portions or parts of these transgenic plants are also provided. Portions and parts of plants includes, but is not limited to, plant cells, plant tissue, plant progeny, plant asexual propagates, plant seeds.

Transgenic plant cells comprising a polynucleotide encoding a polypeptide capable of increasing resistance to nematodes such as SCN are also provided. Suitably the plant cells are soybean plant cells. Suitably these cells are capable of regenerating a plant. The polypeptide comprises the sequences of SEQ ID NOs:10-18, or fragments, variants or combinations thereof. The polypeptide can be 70%, 75%, 85%, 90%, 95%, 97%, 98%, 99% or 100% identical to the sequences provided. The transgenic cells can be found in a seed. A plant, such as a soybean plant, can include the transgenic cells. The plant can be grown from a seed comprising transgenic cells or can be grown by any other means available to those of skill in the art. Chimeric plants comprising transgenic cells are also provided.

Expression of polypeptides and polynucleotides encoding the polypeptides in the transgenic plant is altered relative to the level of expression of the native polypeptides in a control soybean plant. In particular the expression of the polypeptides in the root of the plant is increased. The transgenic plant has increased resistance to nematodes as compared to the control plant. The transgenic plant can be generated from a transgenic cell or callus using methods available to those skilled in the art.

### EXAMPLES

The Examples that follow are illustrative of specific embodiments disclosed herein and various uses thereof. They are set forth for explanatory purposes only and are not to be taken as limiting.

### Example 1: Abundance of WT and Resistance-Associated α-SNAP Proteins in Rhg1_{HC} and Rhg1_{LC} Soybean Varieties

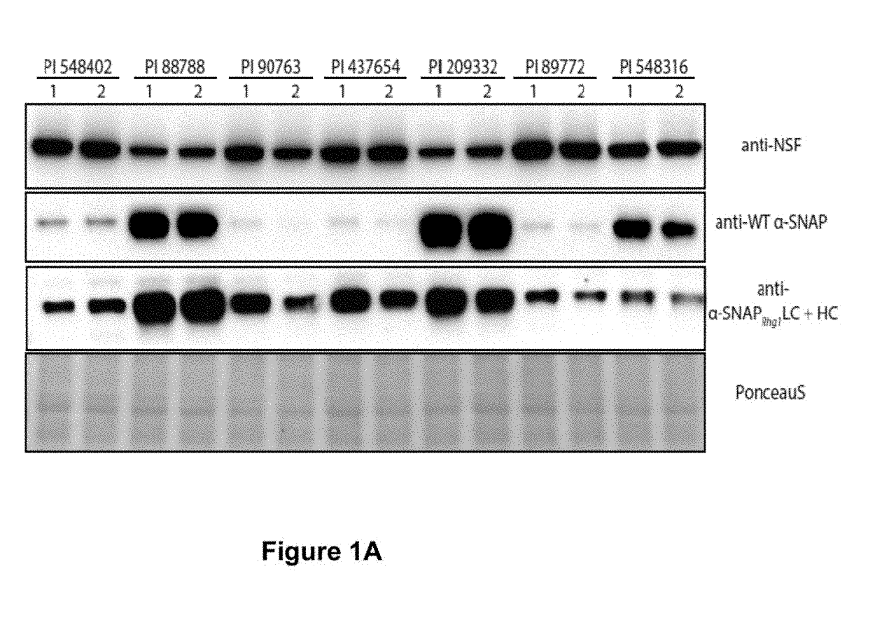

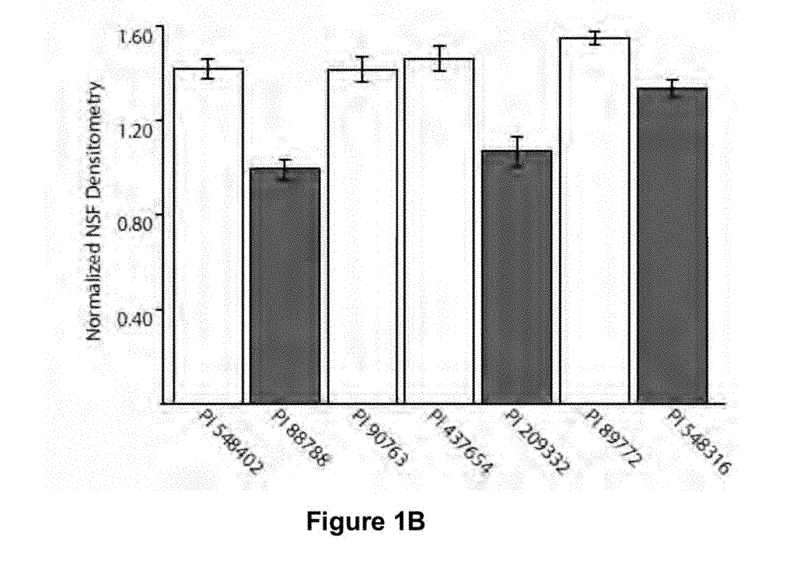

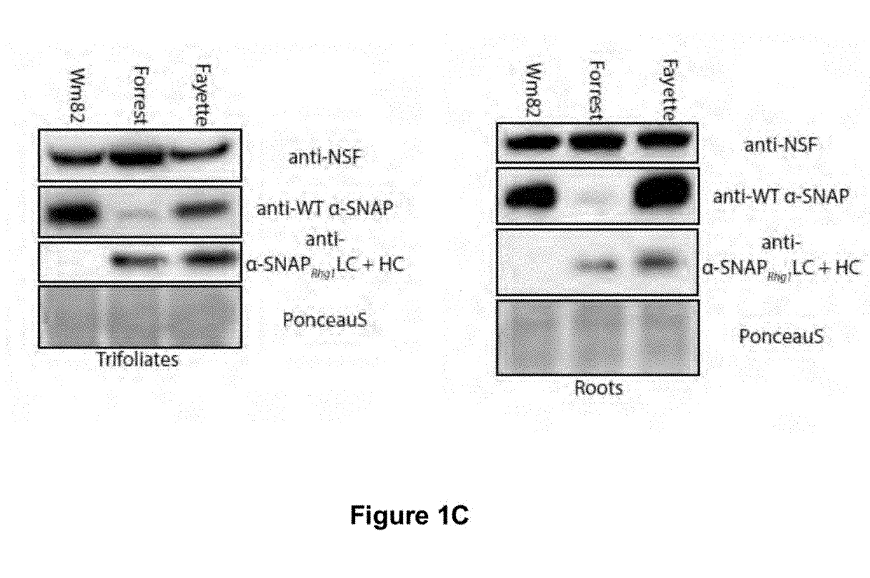

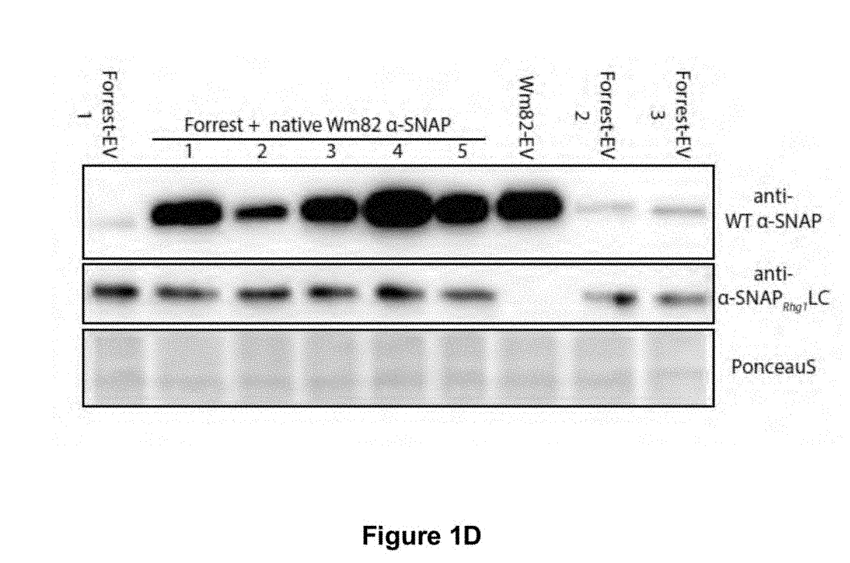

To investigate the relative abundances of wildtype (WT) and resistance-associated α-SNAPs, immunoblots were performed using standard HG type test Rhg1_{HC} and Rhg1_{LC} soybean varieties and previously described anti-α-SNAP antibodies (Niblack et al., 2002, J Nematol 34, 279-288; Bayless et al., 2016, Proc. Natl. Acad. Sci. USA 113, E7375-E7382). NSF abundance was also studied in these samples using an antibody raised to a conserved NSF domain. As shown in FIG. 1A, immunoblots from root tissue indicated that WT α-SNAP abundance in all tested Rhg1_{LC} lines (PI 548402/Peking, PI 90763, PI 437654, PI 89772) was dramatically reduced compared with the Rhg1_{HC} lines (PI 88788, PI 209332, PI 548316). Probing of the same samples with antibodies that recognize α-SNAP_{Rhg1}LC or α-SNAP_{Rhg1}HC but not WT α-SNAP confirmed that, between the Rhg1_{HC} and Rhg1_{LC} soybean varieties, there was a pronounced difference in the abundance of WT α-SNAP relative to the abundance of Rhg1 α-SNAP (FIG. 1A).

WT α-SNAP expression was similarly reduced in a more recent agriculturally utilized Rhg1_{LC} soybean variety, “Forrest.” Immunoblots on both total leaf or root proteins from Williams82 (Rhg1 single copy), Forrest (Rhg1_{LC}) and Fayette (Rhg1_{HC}), again revealed sharp decreases in total WT α-SNAP abundance in the Rhg1_{LC} source Forrest (FIG. 1C). Altogether, a sharply reduced total abundance of WT α-SNAPs was observed to be a shared trait of Rhg1_{LC} soybean varieties but not Rhg1_{HC} varieties. This strikingly low abundance of WT α-SNAPs is likely due to the absence of a WT-α-SNAP-encoding allele at Rhg1_{Lc}, low or no product from the Glyma.11G234500 (α-SNAP_{Ch11}) allele containing an intronic splice site mutation, and a relatively low contribution of protein from the other three putative α-SNAP-encoding loci (Table 1.)

Table 1: Normalized RNA seq reads for soybean α-SNAP transcripts from Williams82

TABLE 1
Normalized RNA seq reads for soybean α-SNAP transcripts from Willams82
Normalized RNA seq reads for soybean α-SNAP transcripts from Willams82

| | | | one | pod | pod | | | | | | | | | |
|---|---|---|---|---|---|---|---|---|---|---|---|---|---|---|
| alpha-SNAP | young_ | | cm | shell | shell | seed | seed | seed | seed | seed | seed | seed | | |
| gene | leaf | flower | pod | 10DAF | 14DAF | 10DAF | 14DAF | 21DAF | 25DAF | 28DAF | 35DAF | 42DAF | root | module |
| Glyma02g42820 | 0 | 0 | 0 | 0 | 0 | 0 | 1 | 2 | 1 | 0 | 1 | 0 | 0 | 0 |
| Glyma09g41590 | 4 | 4 | 3 | 2 | 2 | 1 | 1 | 2 | 2 | 1 | 1 | 1 | 10 | 11 |
| Glyma11g35820 | 16 | 17 | 20 | 23 | 26 | 13 | 17 | 11 | 14 | 6 | 15 | 10 | 22 | 12 |
| Glyma14g05920 | 0 | 5 | 3 | 2 | 1 | 10 | 6 | 2 | 1 | 1 | 1 | 2 | 1 | 9 |
| Glyma18g02590 | 26 | 28 | 32 | 44 | 24 | 21 | 27 | 9 | 13 | 7 | 12 | 7 | 28 | 10 |

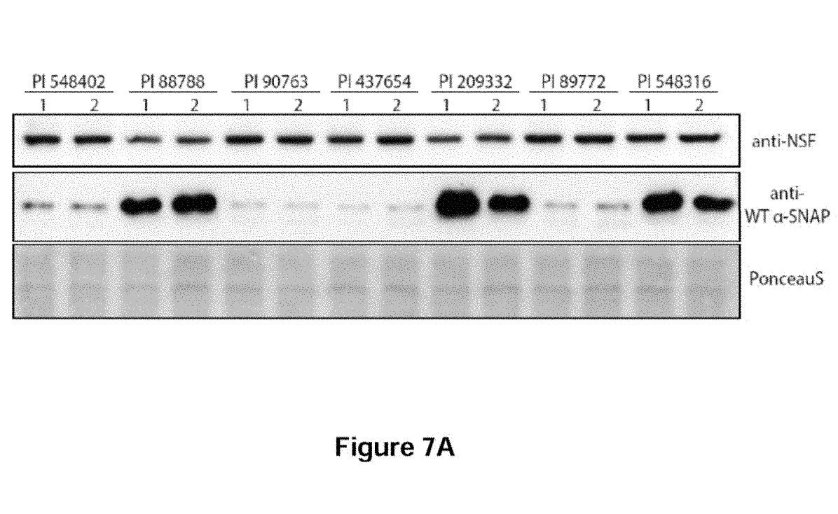

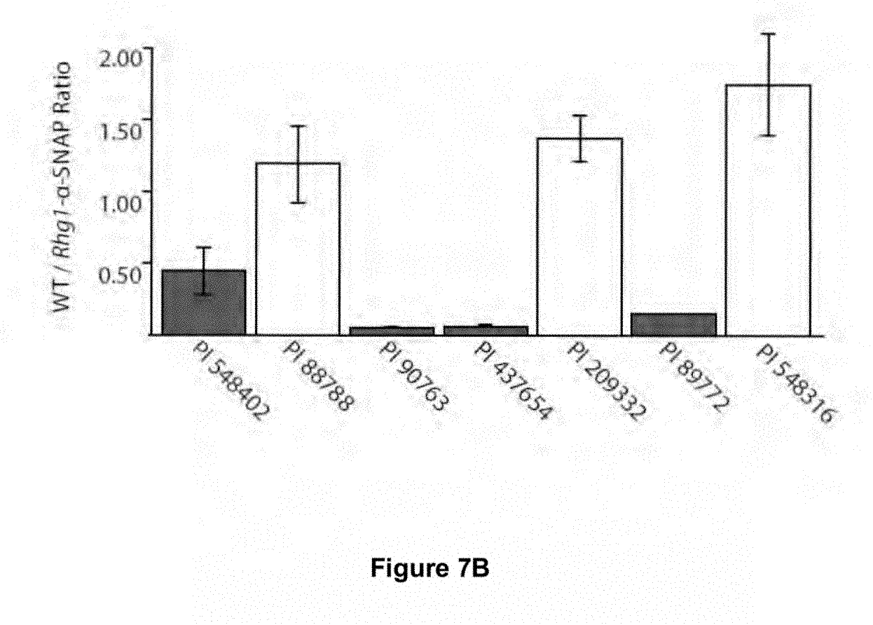

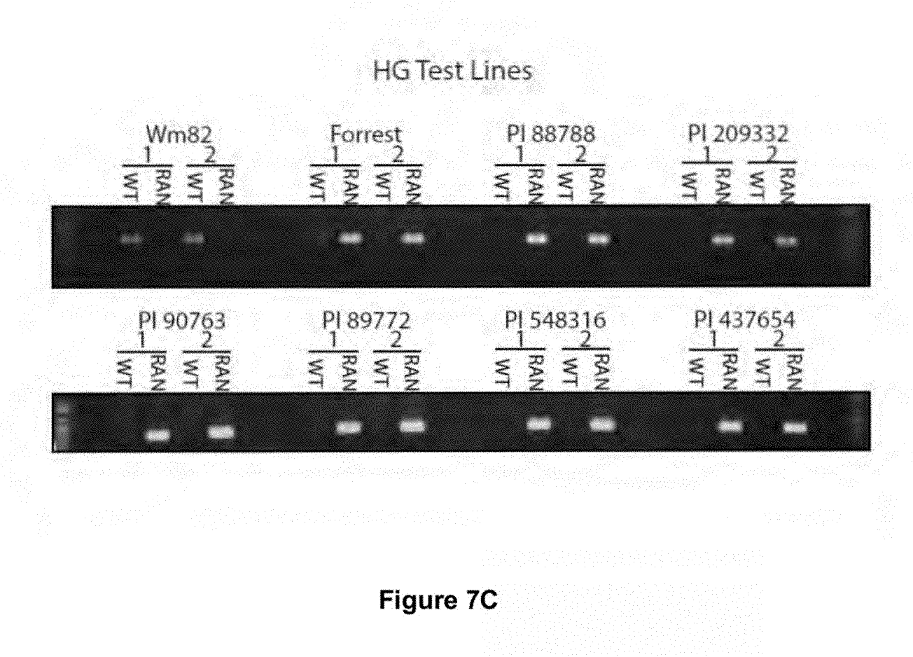

NSF protein abundance in the Rhg1_{LC} lines was increased compared with the Rhg1_{HC} lines PI 88788 and PI 209332 (FIG. 1A, FIG. 7A). In PI 548316, which carries only 7 copies of Rhg1_{HC} and encodes an interrupted Chromosome 11 α-SNAP, total NSF expression was more similar to the Rhg1_{LC} lines (FIG. 1A, 7A). These differences in NSF expression, across two independent experiments, were quantified using densitometry with ImageJ (FIG. 1B)

Whether native α-SNAP_{Rhg1}WT locus, if expressed, could contribute to total WT α-SNAP protein abundance in Rhg1_{LC} soybean lines was also investigated. Cloning native Glyma.18G022500 α-SNAP_{Rhg1}WT locus from Williams 82 (Wm82), transgenic Forrest (Rhg1_{Lc}) roots expressing native α-SNAP_{Rhg1}WT were generated and total WT α-SNAP abundance was assessed with immunoblots. Compared to empty vector controls, transgenic addition of the native Williams 82 α-SNAP_{Rhg1}WT locus increased wild type α-SNAP abundance in Forrest to levels similar to Williams 82 controls (FIG. 1D).

### Example 2: A Unique NSF_{Ch07} Allele (RAN07) is Present in Rhg1-Containing NAM Parents and HG Type Test Type Varieties

Rhg1-resistance type α-SNAPs (α-SNAP_{Rhg1}LC or α-SNAP_{Rhg1}HC) exhibited compromised binding to wild-type NSFs and were toxic at high doses in N. benthamiana (Bayless et al., 2016, Proc. Natl. Acad. Sci. USA 113, E7375-E7382). (NSF and α-SNAP are essential housekeeping proteins in all eukaryotes and null mutations in either partner are lethal in animals, which typically encode only single copies of NSF or α-SNAP (Littleton et al., 2001, 98, 12233-12238; Sanyal and Krishnan, 2001, Neuroreport 12, 1363-1366; Horsnell et al., 2002, Biochemistry 41, 5230-5235; Chae et al., 2004, Nat Genet 36, 264-270).

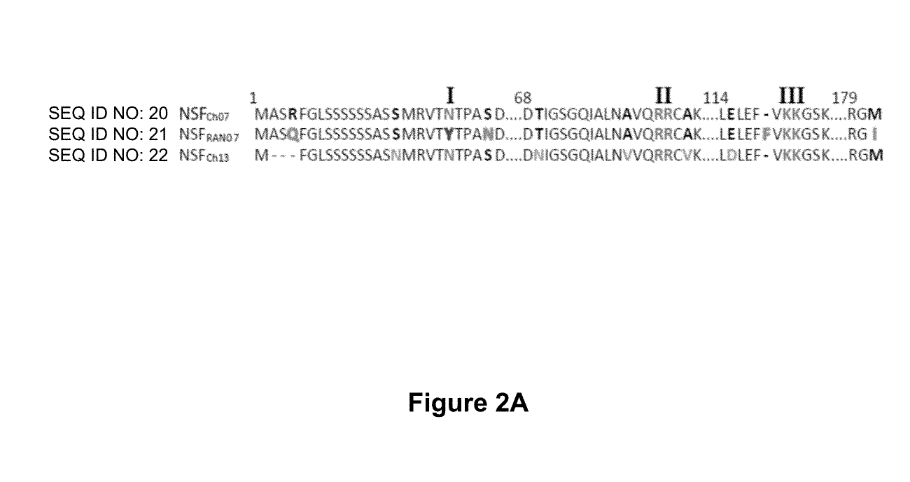

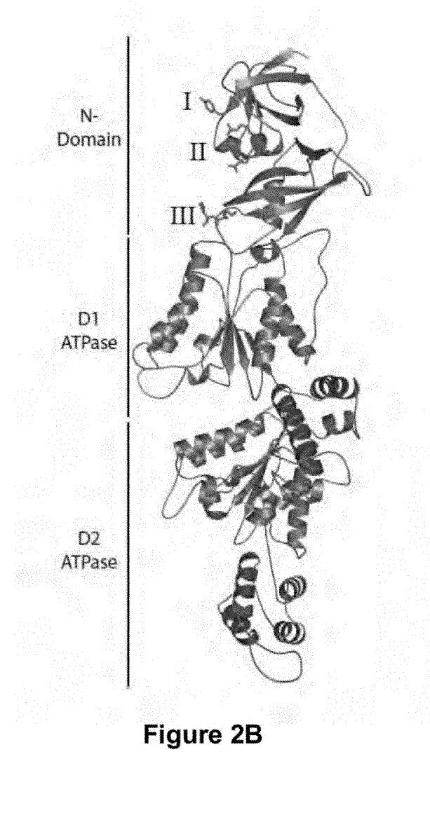

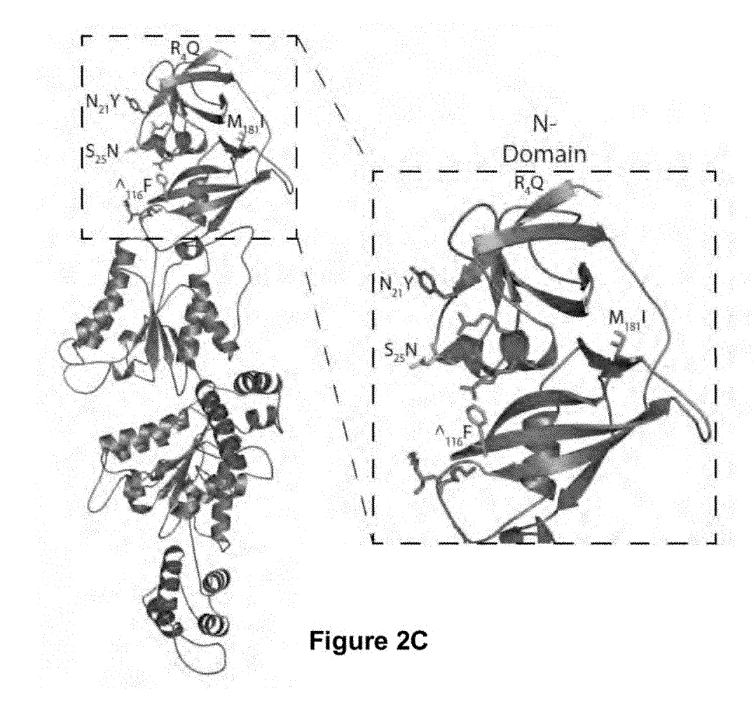

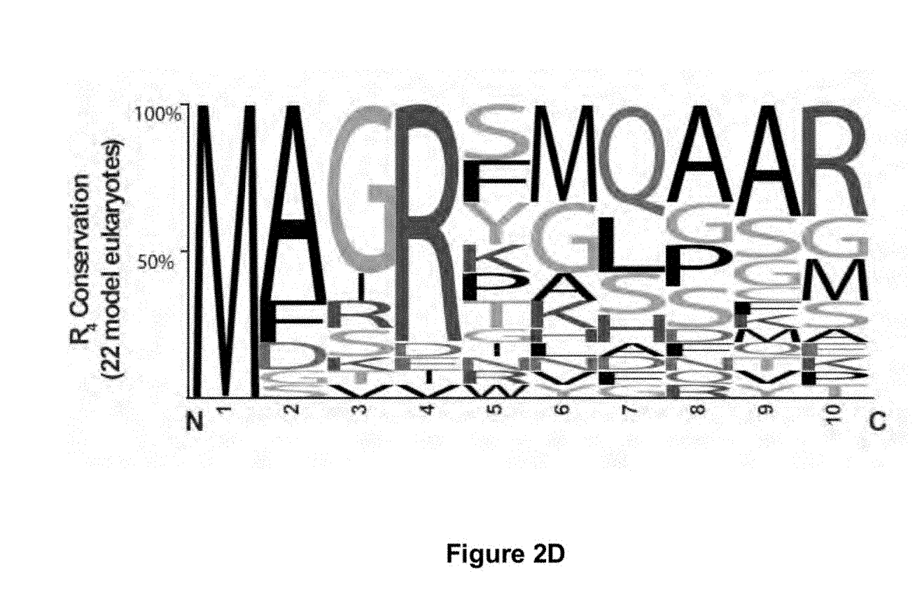

Viability of plants harboring Rhg1-resistance type α-SNAP_{Rhg1}LC was investigated by examining alternative sources of α-SNAP or NSF activity. Soybean is a polyploid organism encoding multiple α-SNAP and NSF loci. Alterations in other α-SNAP (Glyma.11G234500, Glyma.14G054900, Glyma.02G260400, Glyma.09G279400) or NSF loci (Glyma.13G180100) were examined using whole genome sequence (WGS) data from multiple Rhg1-containing varieties. Briefly, reads were assembled for all α-SNAP and NSF loci, and aligned against the Williams 82 reference genome. In all α-SNAP loci from Rhg1_{LC} varieties, no obvious polymorphisms were detected other than the previously reported Glyma.11G234500 (a-SNAPch 11) allele containing an intronic splice site mutation. (Cook, 2014, Plant Physiol 165, 630-647) Among all examined Rhg1Lc and Rhg1Hc lines, a novel NSFcho1 allele was present containing five N-Domain amino acid polymorphisms (R4Q, N21Y, S25 N, A 116F, M1811) (FIG. 2A).

Using cDNA from Forrest (Rhg1_{LC}), this unique NSF_{Ch07} transcript was cloned and sequenced, and all 5 N-domain polymorphisms were confirmed. Additionally, two different PCR primer pairs were designed at the N₂₁Y and S₂₅N polymorphisms and this unique NSF_{Ch07} allele (and absence of the wild-type NSF_{Ch07}allele) was verified in all HG type test lines using agarose gel electrophoresis (FIG. 7C).

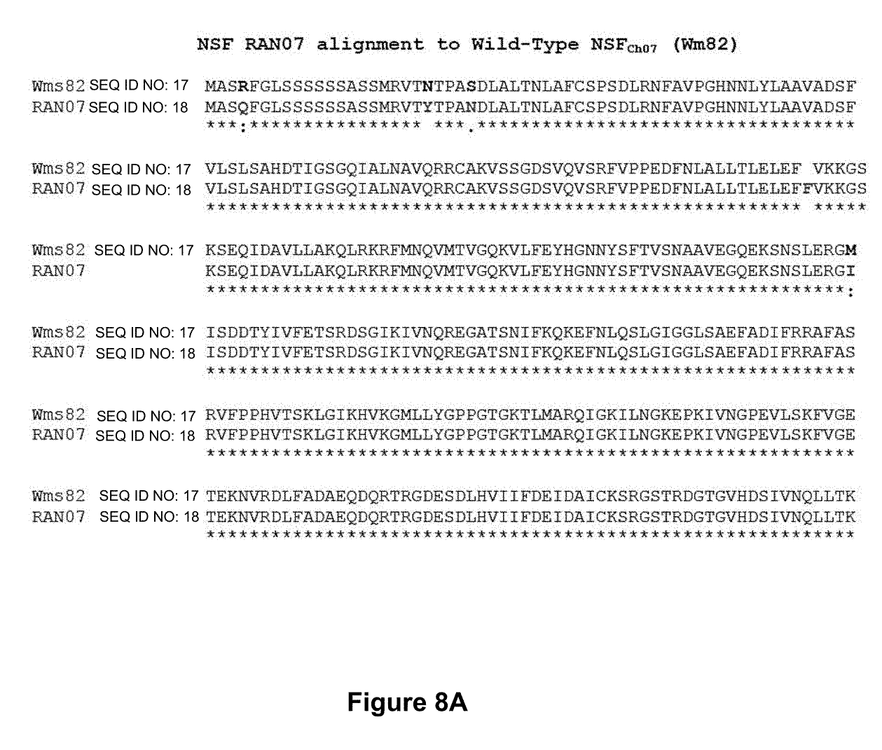

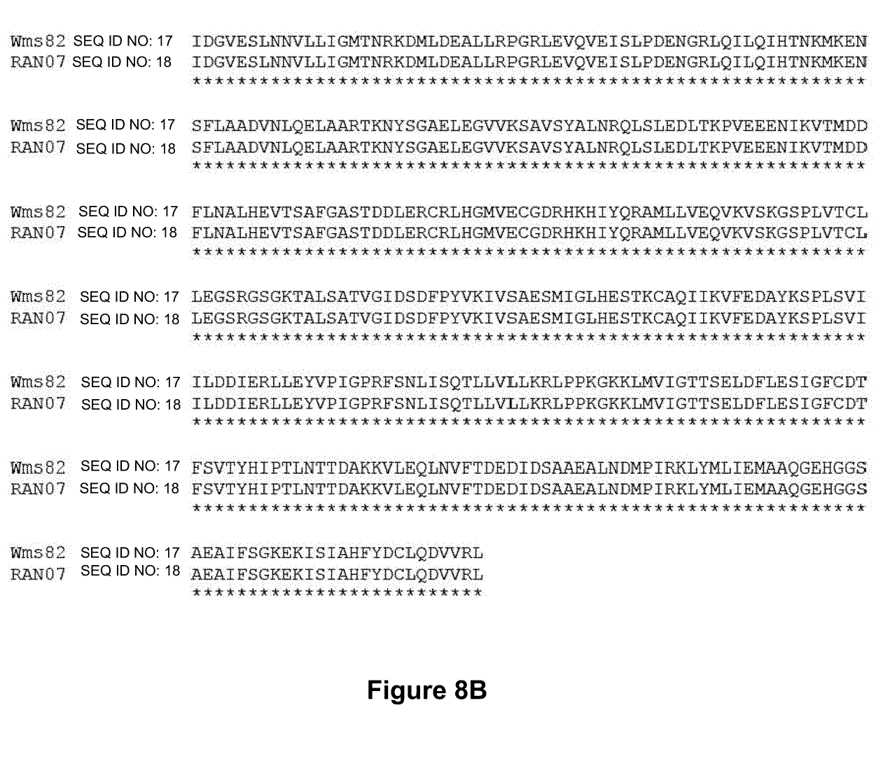

Whole genome sequencing (WGS) data from the SoyNAM (Nested Association Mapping) project (Song et al., 2017b, Plant Genome 10(2)) was used to determine that this unique NSF_{Ch07} allele was in every Rhg1-containing NAM parent, while SCN-susceptible NAM parents carried the WT NSF_{Ch07} allele (Table 1). The protein from this Rhg1-associated allele of Glyma.07G195900 was designated “NSF_{RAN07}” for “Rhg1-associated NSF from chromosome 07.” In addition to NSF_{RAN07}, an allele of the chromosome 13 Glyma.13g180100 gene encoding an NSF_{Ch13} V₅₅₅I protein was found in some varieties, including SCN-susceptible soybeans, but it was not present in all Rhg1_{LC} or Rhg1_{HC} lines (Table 2). FIG. 8A and FIG. 8B shows the complete NSF_{RAN07} amino acid alignment to NSF_{Ch07} from the Williams 82 reference genome.

TABLE 2
HG Type Test lines and Rhg1-containing NAM
Parents Contain a Unique NSF_{Ch07} Allele

| Line | Rhg1 Haplotype | NSF_{Ch07} | NSF_{Ch13} |
|---|---|---|---|
| Peking | Rhg1_{LC} | Rhg1 Assoc. Allele | WT (Wm82-type) |
| 90763 | Rhg1_{LC} | Rhg1 Assoc. Allele | V555I |
| 437654 | Rhg1_{LC} | Rhg1 Assoc. Allele | WT (Wm82-type) |
| 209332 | Rhg1_{HC} | Rhg1 Assoc. Allele | V555I |
| 89772 | Rhg1_{LC} | Rhg1 Assoc. Allele | V555I |
| 548316 | Rhg1_{HC} | Rhg1 Assoc. Allele | V555I |
| Prohio | Susceptile | WT (Wm82-type) | V555I |
| NE3001 | Susceptile | WT (Wm82-type) | Y260F |
| 4J105-34 | Rhg1_{HC} | Rhg1 Assoc. Allele | V555I, L738F |
| CL0J095-46 | Rhg1_{HC} | Rhg1 Assoc. Allele | V555I |
| IA3023 | Susceptible | WT (Wm82-type) | V555I |
| LD00-3309 | Rhg1_{HC} | Rhg1 Assoc. Allele | WT (Wm82-type) |
| LD02-4485 | Rhg1_{HC} | Rhg1 Assoc. Allele | WT (Wm82-type) |
| LG05-4292 | Rhg1_{HC} | Rhg1 Assoc. Allele | WT (Wm82-type) |
| LD01-5907 | Rhg1_{LC} | Rhg1 Assoc. Allele | V555I |
| LD02-9050 | Rhg1_{HC} | Rhg1 Assoc. Allele | V555I |
| Magellan | Susceptible | WT (Wm82-type) | WT (Wm82-type) |
| Maverick | Rhg1_{HC} | Rhg1 Assoc. Allele | V555I |

### Example 3: NSF_{RAN07} and Rhg1 α-SNAP Polymorphisms are Both at the NSF/α-SNAP Binding Interface

The NSF/α-SNAP interface consists of complementary electrostatic patches at the NSF N-domain and α-SNAP C-terminus (Zhao and Brunger, 2016, J Mol Biol 428, 1912-1926). These binding patches are conserved in yeast, animals and plants, with the soybean NSF N-domain (N₂₁, RR₈₂₋₈₃, KK₁₁₇₋₁₁₈) and α-SNAP C-terminus (D₂₀₈DEED₂₄₃₋₂₄₆, EEDD₂₈₄₋₂₈₇) corresponding to NSF_{CHO} (R₁₀, RK₆₇₋₆₈, KK₁₀₄₋₁₀₅) and rat α-SNAP (D₂₁₇E₂₄₉EE₂₅₂₋₂₅₃, DEED₂₉₀₋₂₉₃) respectively. Accordingly, inter-kingdom interactions between α-SNAP and NSF have been reported both in vitro and for heterologous expression systems in vivo, including between soybean WT α-SNAP and Chinese Hamster NSF (NSF_{CHO}) (Griff et al., 1992, J. Biol. Chem. 267, 12106-12115; Bassham and Raikhel, 1999, Plant J 19, 599-603; Rancour et al., 2002, Plant Physiol 130, 1241-1253; Bayless et al., 2016, Proc. Natl. Acad. Sci. USA 113, E7375-E7382).

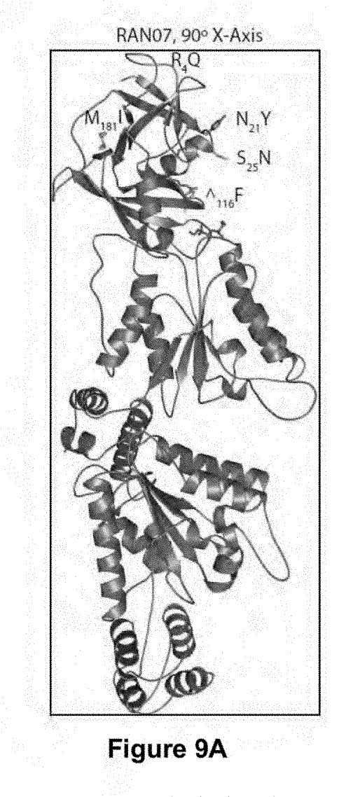

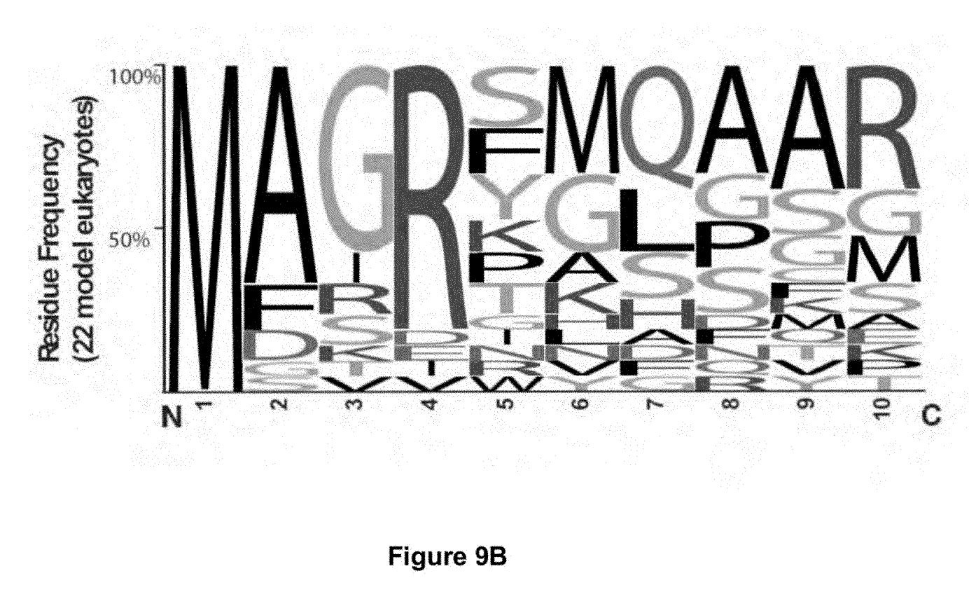

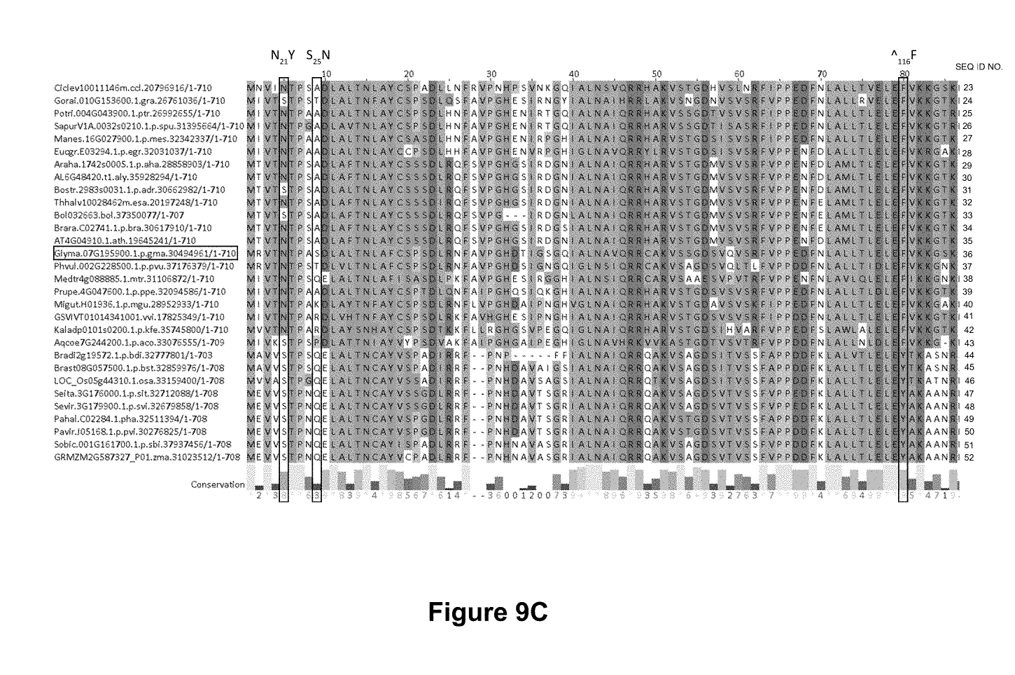

To assess where the NSF_{RAN07} polymorphisms are positioned in the N-domain, NSF_{RAN07} was modeled to the NSF_{CHO} cryo-EM structure from Zhao and colleagues (Zhao, 2015, Nature 518, 61-67) (FIG. 2B). NSFs in many plants, including soybean, encode a variable length polyserine/glycine patch, starting at ˜residue 6. Therefore, modeling to NSF_{CHO} began at residue 14. The NSF_{RAN07} homology model to NSF_{CHO} placed two of the NSF_{RAN07} polymorphisms at two NSF_{CHO} regions that bind α-SNAP: N₂₁Y and S₂₅N at and near R₁₀, and ^{\hat{ }}₁₁₆F at RK₁₁₄₋₁₁₅, respectively (FIG. 2B, FIG. 2C, FIG. 9A). While R₄Q was omitted from the model (because of the omission of the variable length polyserine/glycine patch), we examined R₄ frequency across 22 diverse eukaryotes (9 animals, 3 fungi, 10 plants) (FIG. 2D). In all but four model organisms, R₄ was present in the NSF of 18 of the 22 species, while S. cerevisiae, Drosophila, C. elegans and Physcomitrella carry an R and/or K at the adjacent residue #3 and/or #5. The final NSF_{RAN07} polymorphism, M₁₈₁I, was not located near the α-SNAP binding patches and was not highly conserved among model organism NSFs. Examination of N-domain conservation in plant NSFs revealed that residues corresponding to N₂₁ and F₁₁₅ are present in a majority of plants and do not carry N₂₁Y or the ^{\hat{ }}₁₁₈F insertion (FIG. 9B). These results modeling to NSF demonstrate that three of the five NSF_{RAN07} N-domain polymorphisms are located in or adjacent to the NSF binding patches that interact with α-SNAP.

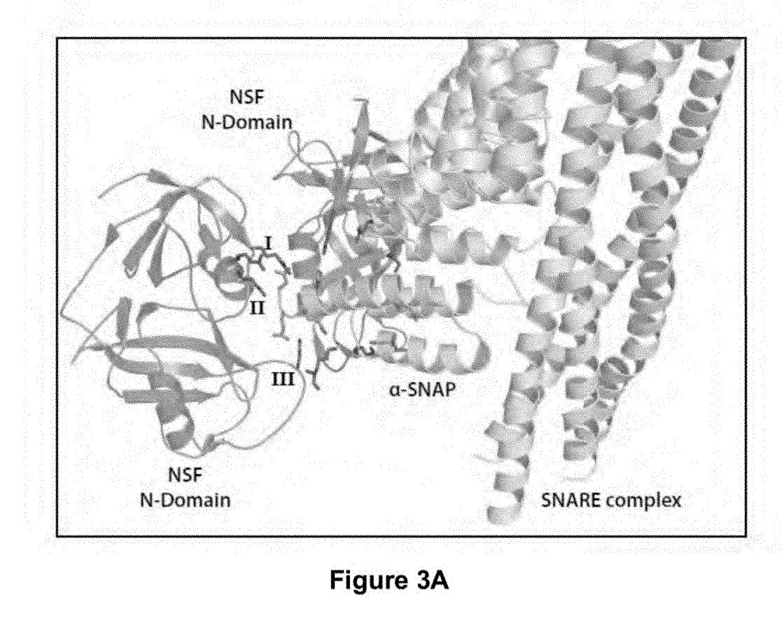

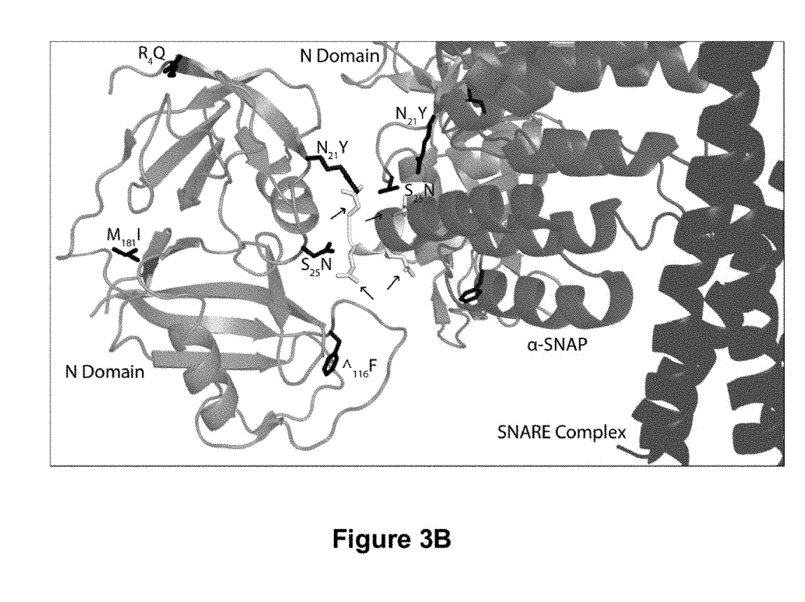

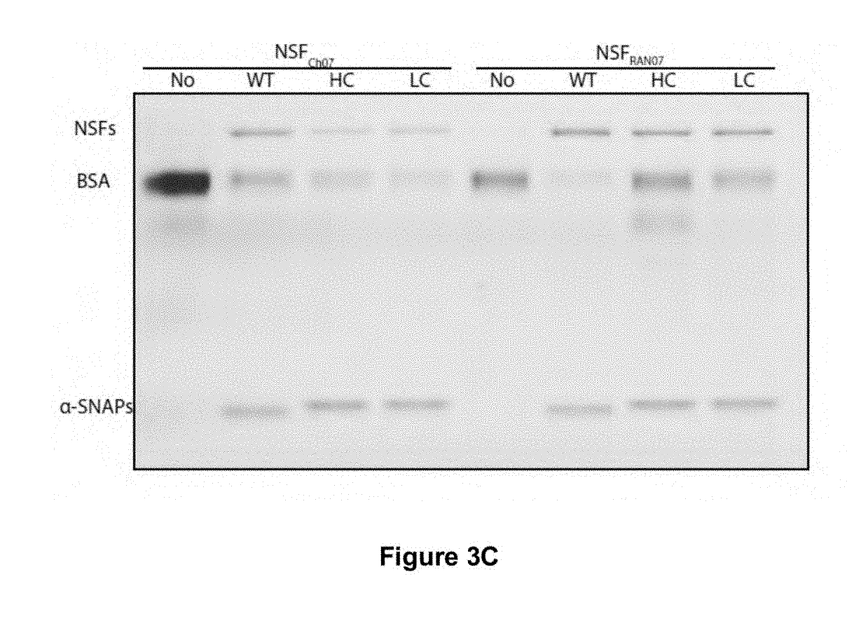

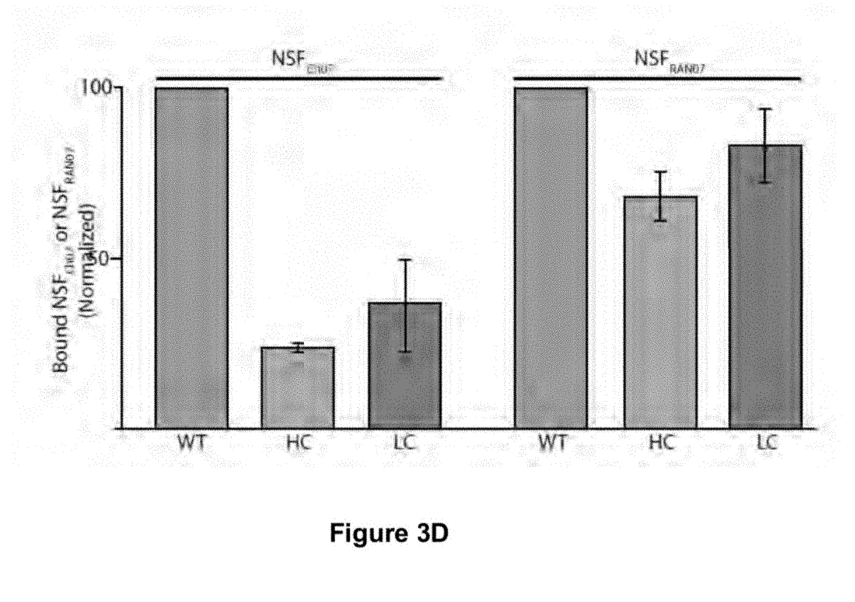

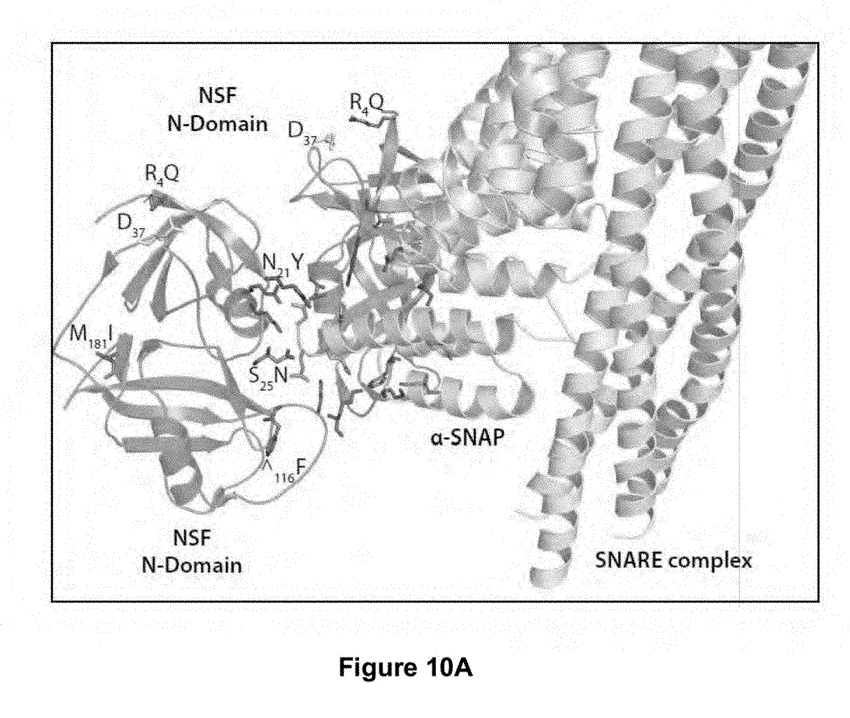

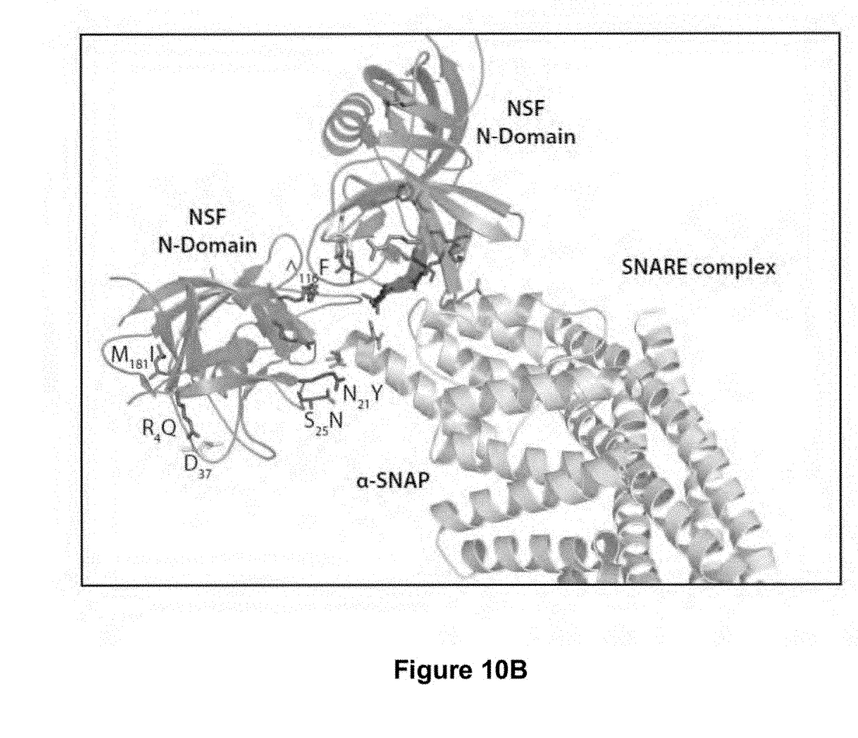

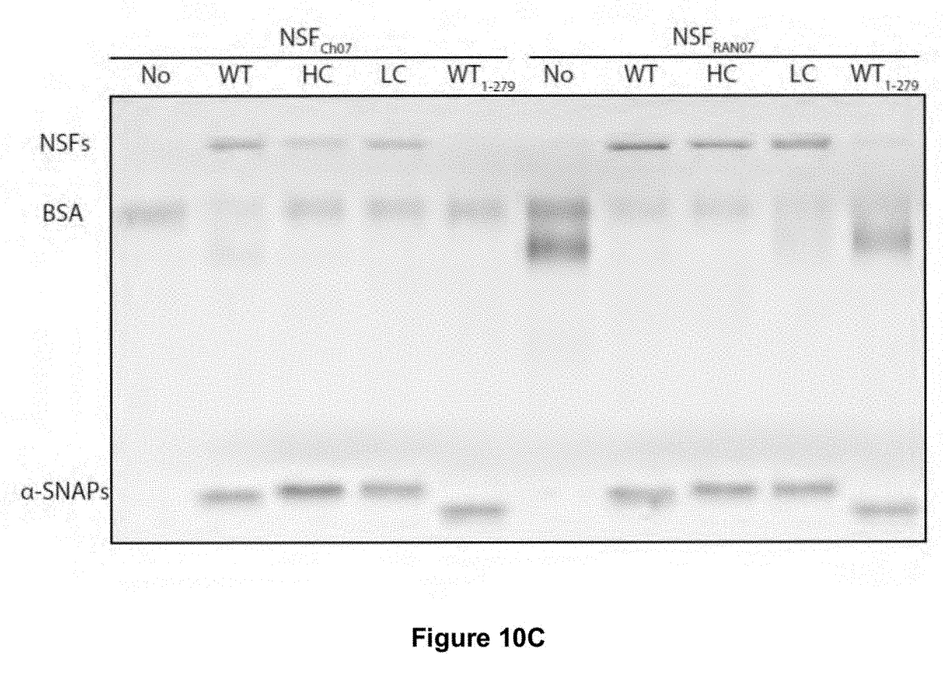

Polymorphisms of both α-SNAP_{Rhg1}HC and α-SNAP_{Rhg1}LC, are located at conserved C-terminal residues that bind and stimulate NSF (Cook et al., 2014, Plant Physiol 165, 630-647; Bayless et al., 2016, Proc. Natl. Acad. Sci. USA 113, E7375-E7382). Multiple α-SNAP proteins bound to a SNARE bundle recruit six NSF proteins to form a “20S supercomplex” (4× α-SNAPs, 6×NSF, 3-4×SNAREs) and stimulate SNARE complex disassembly (Zhao et al., 2015). The proximity of the NSF_{RAN07} N-domain polymorphisms to α-SNAP C-terminal contacts was assessed by identifying and coloring the complementary NSF and α-SNAP binding residues, and then the NSF_{RAN07} and Rhg1 α-SNAP polymorphisms, on the mammalian 20S cryo-EM structure (FIG. 3A, FIG. 3B, FIG. 10A, FIG. 10B). This confirmed that NSF_{RAN07} N₂₁Y, S₂₅N, ^{\hat{ }}₁₁₆F are predicted to locate adjacent to NSF residues that bind α-SNAP residues, including residues that contact the WT α-SNAP amino acid residues that are altered in α-SNAP_{Rhg1}HC and α-SNAP_{Rhg1}LC. R₄ on the NSF_{CHO} structure was closely positioned to a D₂₈ side chain, present in soybean as D₃₉ (FIG. 10B). Altogether, the location and structural modeling of the NSF_{RAN07} polymorphisms suggest that NSF_{RAN07} modifies the normal NSF binding interface that maintains complementary binding contacts with α-SNAP sites that are altered in Rhg1 α-SNAPs.

### Example 4: NSF_{RAN07} Polymorphisms Promote Binding with Rhg1 Resistance-Type α-SNAPs

All Rhg1-containing HG type test and NAM lines contained NSF_{RAN07}, and α-SNAP_{Rhg1}HC and α-SNAP_{Rhg1}LC are polymorphic at C-terminal residues that bind and stimulate NSF. Therefore, the impact of NSF_{RAN07} polymorphisms on binding to both Rhg1 resistance-type α-SNAPs and α-SNAP_{Rhg1}WT was investigated. Recombinant NSF_{RAN07}, NSF_{Ch07} and Rhg1 α-SNAP proteins were produced for in vitro binding studies as previously described in (Barnard et al., 1997, J Cell Biol 139, 875-883; (Bayless et al. 2016, Proc. Natl. Acad. Sci. USA 113, E7375-E7382). NSF_{RAN07} and NSF_{Ch07} binding was quantified using ImageJ densitometry across three independent experiments (FIG. 3D). NSF_{Ch07} binding to α-SNAP_{Rhg1}HC and α-SNAP_{Rhg1}LC was reduced compared to α-SNAP_{Rhg1}WT (FIG. 3C). In contrast, NSF_{RAN07} binding to α-SNAP_{Rhg1}HC or α-SNAP_{Rhg1}LC was similar to α-SNAP_{Rhg1}WT binding, and was increased ˜30% relative to NSF_{Ch07}.

To verify that NSF_{RAN07}/α-SNAP binding is dependent upon NSF-binding patches at the α-SNAP C-terminus, NSF_{RAN07} binding to an otherwise WT α-SNAP lacking the final 10 C-terminal residues (α-SNAP_{Rhg1}WT₁₋₂₇₉) was determined. Binding of NSF_{Ch07}WT or NSF_{RAN07} binding with α-SNAP _{Rhg1}WT₁₋₂₇₉ was disrupted, similar to the no α-SNAP binding controls (FIG. 10C). Hence NSF_{RAN07}/α-SNAP binding requires the conserved NSF-binding contacts located at the α-SNAP C-terminus. Combined, these binding assays suggested that NSF_{RAN07} not only maintains normal binding to WT α-SNAPs, but also at least partially accommodates the unusual C-terminal NSF-binding interface of Rhg1 resistance-type α-SNAPs.

### Example 5: NSF_{RAN07} Polymorphisms Guard Against Cell Death Induced by Rhg1-Resistance-Type α-SNAP

Transient expression of either α-SNAP_{Rhg1}HC or α-SNAP_{Rhg1}LC in N. benthamiana leaves, via Agrobacterium infiltration, was cytotoxic and elicited hyperaccumulation of the endogenous NSF protein (Bayless et al., 2016 Proc. Natl. Acad. Sci. USA 113, E7375-E7382). Co-expression of WT-α-SNAP with the Rhg1 α-SNAP diminished this toxicity (Bayless et al., 2016 Proc. Natl. Acad. Sci. USA 113, E7375-E7382). The penultimate leucine/isoleucine of α-SNAP, which has been implicated in stimulation of NSF ATPase, was needed for this N. benthamiana cytotoxicity (Bayless et al., 2016, Proc. Natl. Acad. Sci. USA 113, E7375-E7382).

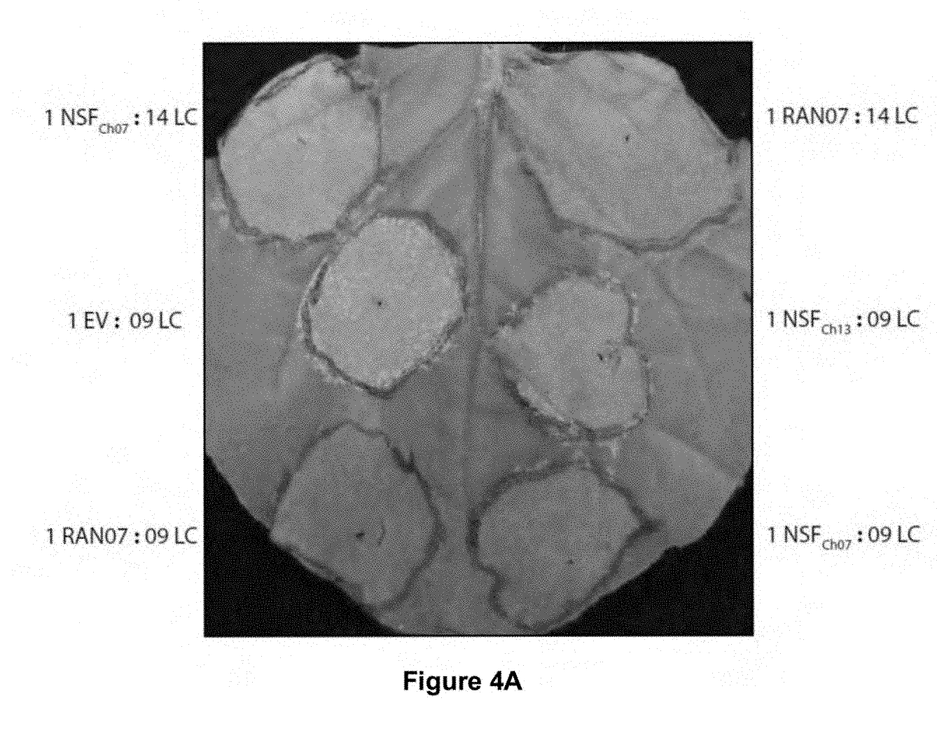

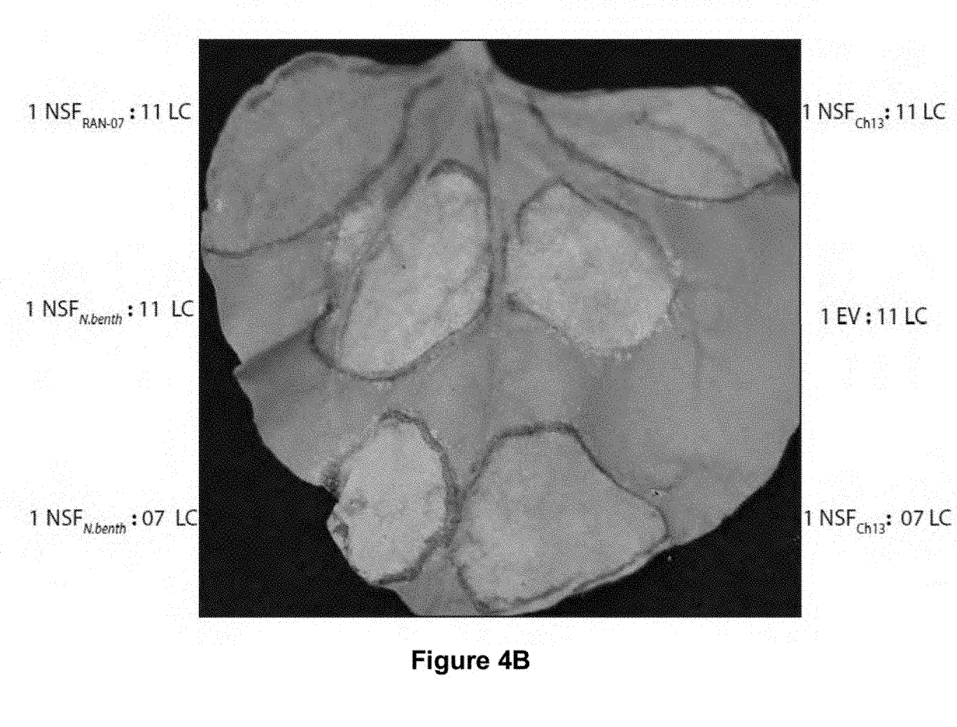

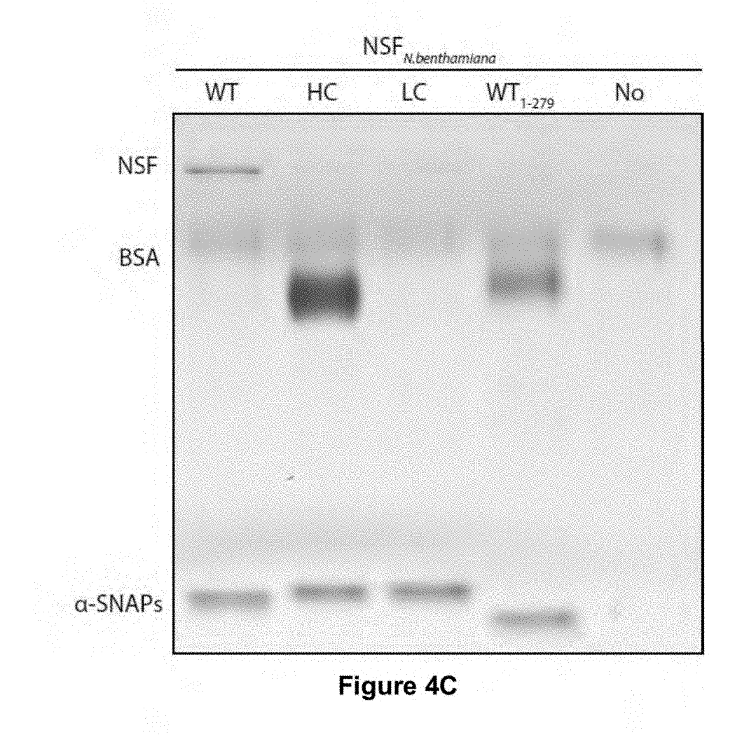

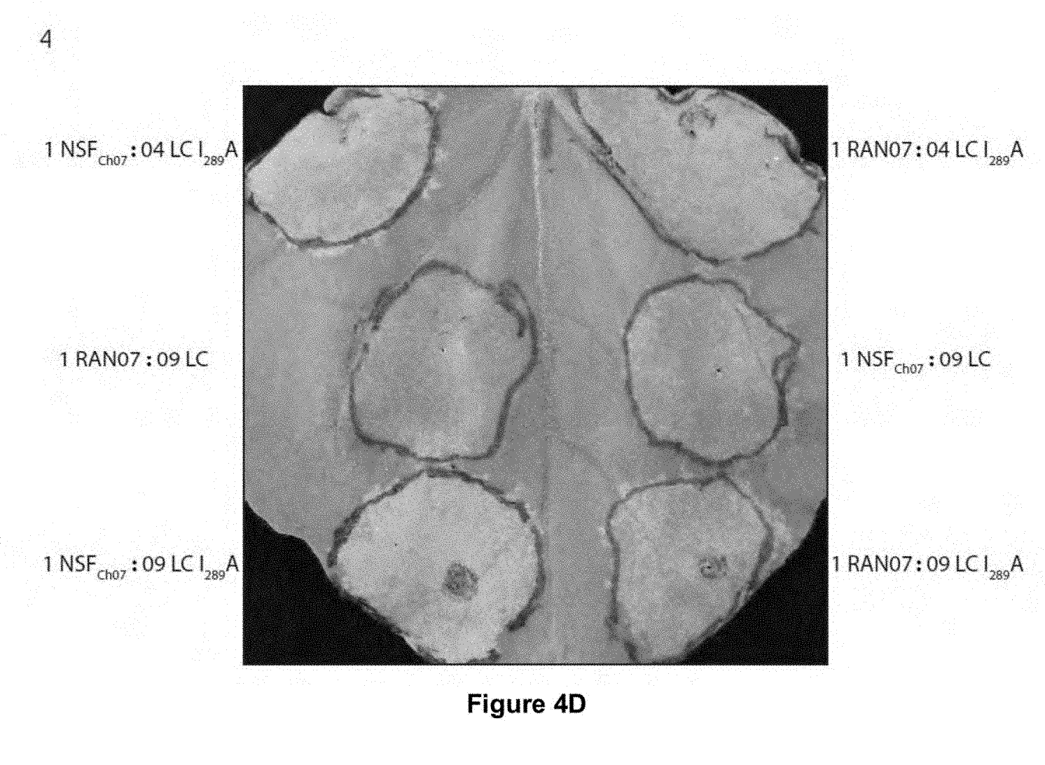

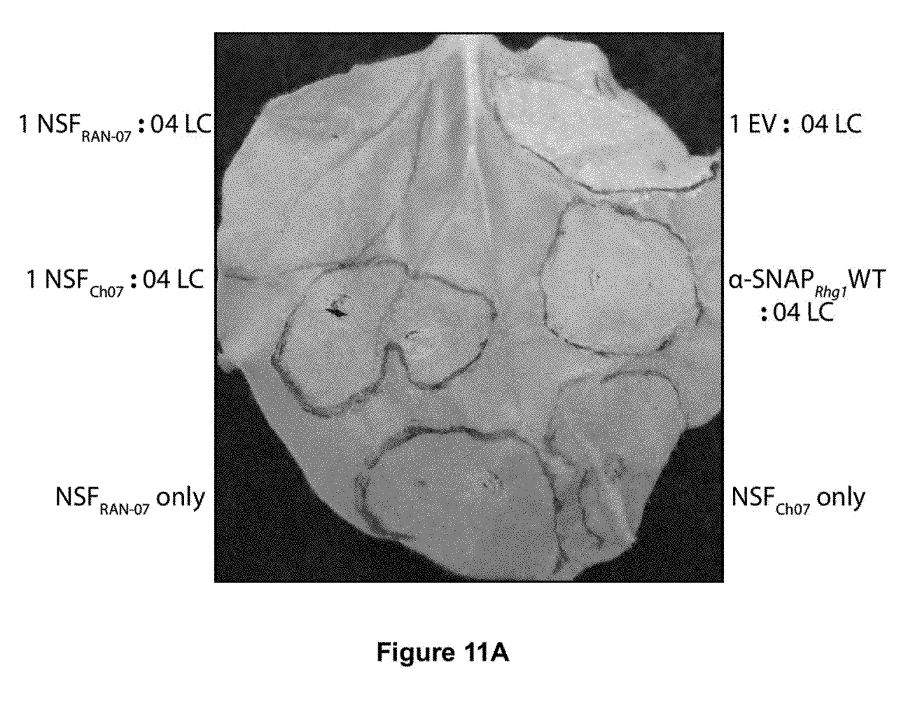

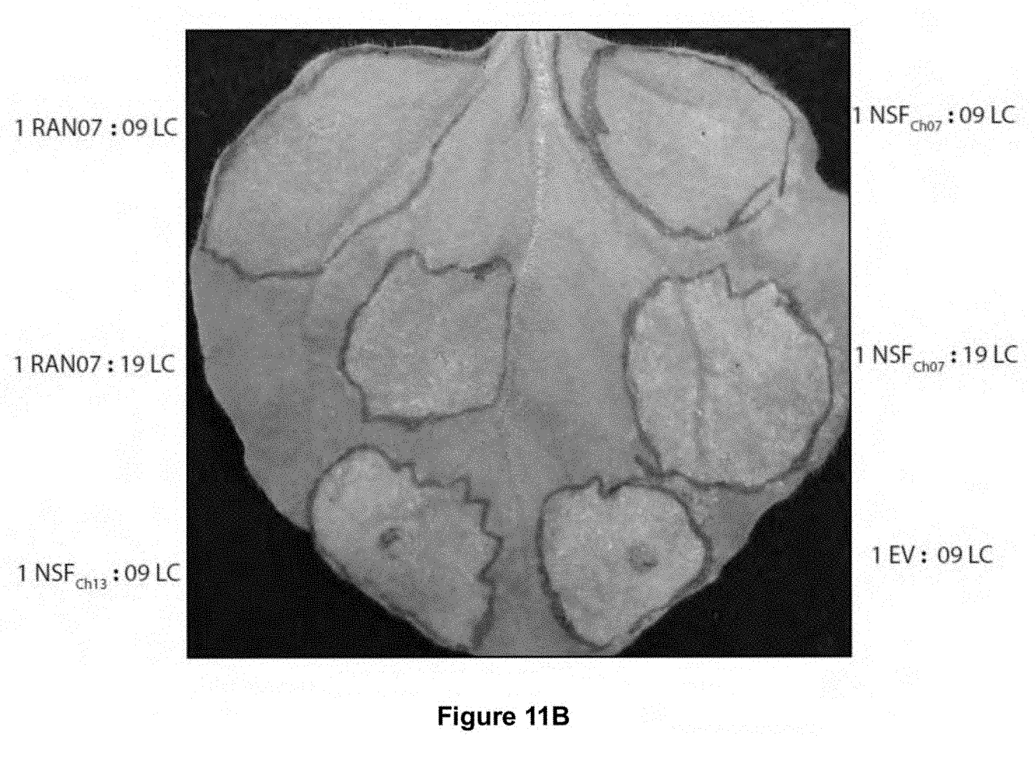

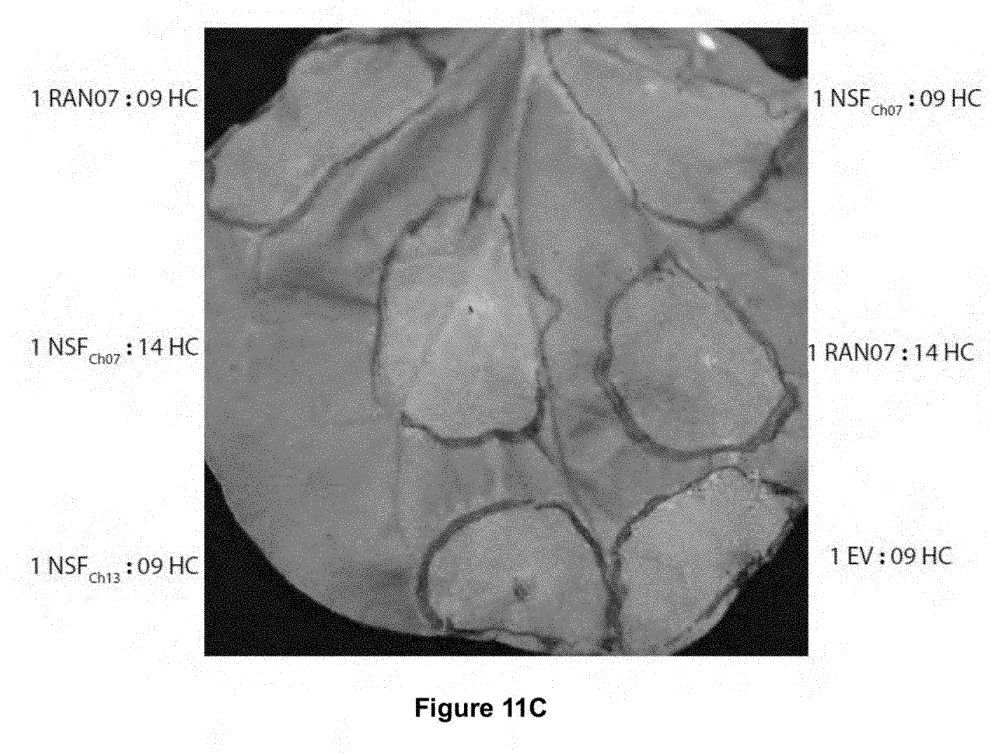

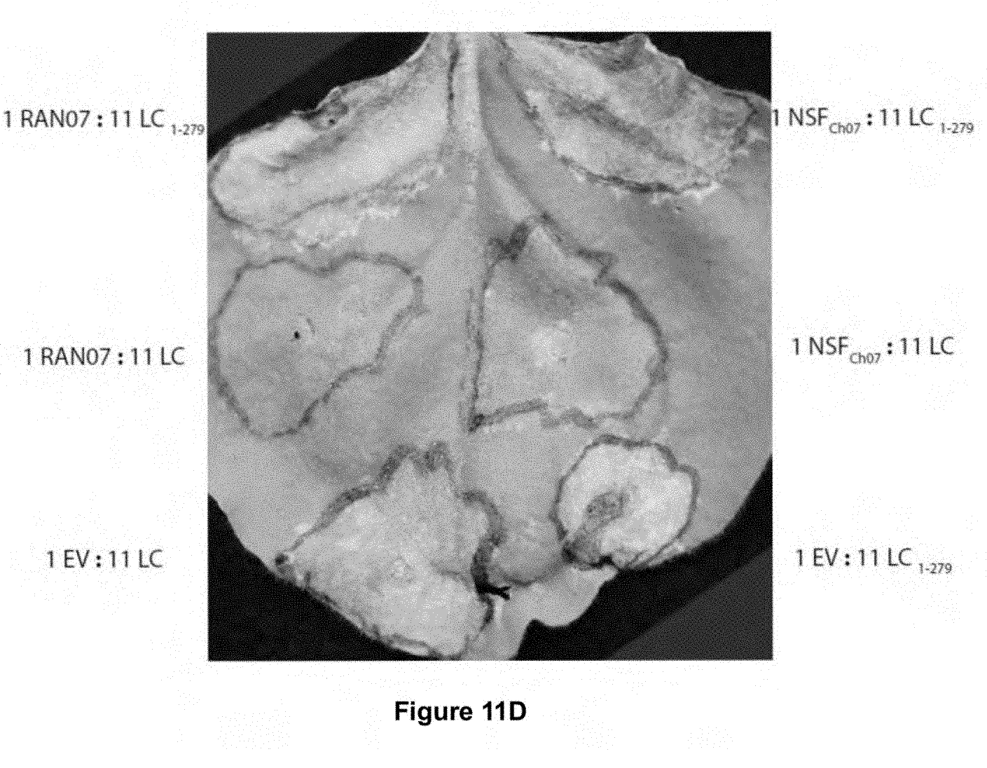

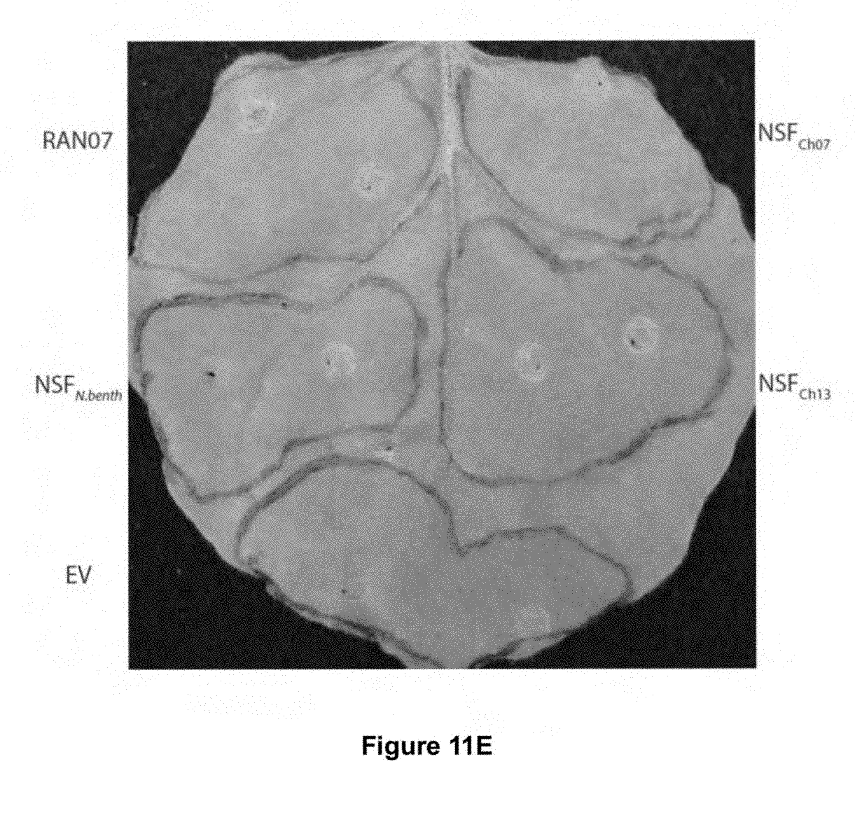

The ability of soybean NSF co-expression to alleviate the toxicity of Rhg1 resistance-type α-SNAPs in N. benthamiana was determined. Mixed Agrobacterium cultures containing 1 part WT α-SNAP to 3 parts α-SNAP_{Rhg1}LC were used for cytotoxicity complementation assays as previously described Bayless et al., 2016, Proc. Natl. Acad. Sci. USA 113, E7375-E7382). NSF_{RAN07} and NSF_{Ch07} were more effective than WT α-SNAP at reducing Rhg1 α-SNAP cytotoxicity (FIG. 11A). The proportion of NSF-delivering bacteria in the mixed Agrobacterium cultures was then decreased down to 1 part to 9 or 14 parts α-SNAP_{Rhg1}LC-delivering bacteria. Co-expressing soybean NSF_{Ch07}, NSF_{Ch13} or NSF_{RAN07} reduced cell death caused by α-SNAP_{Rhg1}LC compared to empty vector controls (FIG. 4A), and NSF_{RAN07} co-expression consistently conferred greater protection than either NSF_{Ch07} or NSF_{Ch13} (FIG. 4A). Infiltrated leaf patches had less death and/or slower death with NSF_{RAN07}. Both NSF_{RAN07} and NSF_{Ch07} were more effective than NSF_{Ch13} at complementing cell death (FIG. 4A). NSF_{RAN07} was observed to confer at least partial protection out to a 1:19 mixture, again outperforming complementation by NSF_{Ch07} (FIG. 11B). Complementation of α-SNAP_{Rhg1}HC-induced cell death with NSF_{RAN07} vs. NSF_{Ch07} produced similar results (FIG. 11C).

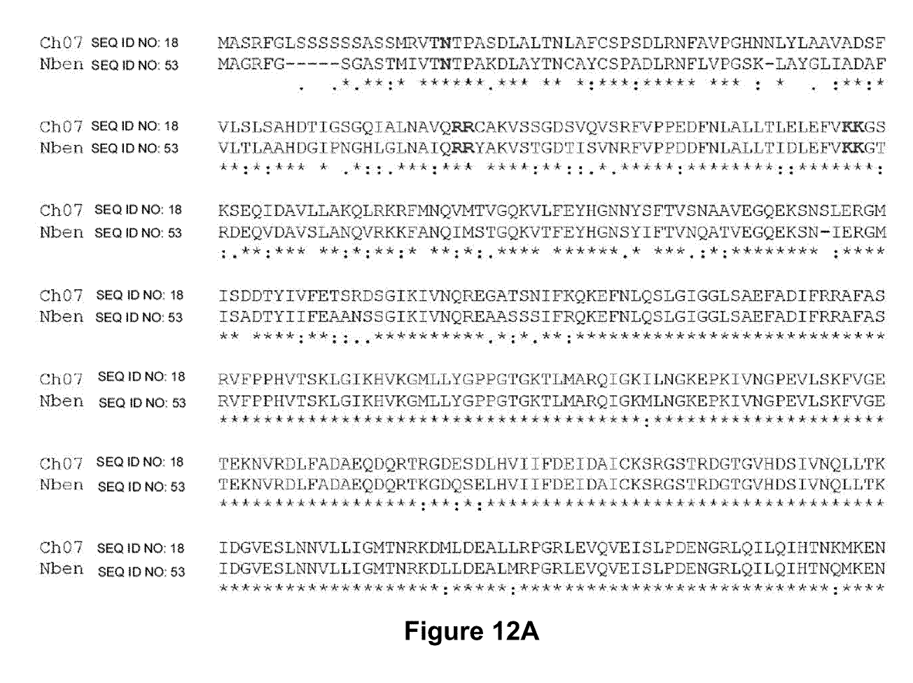

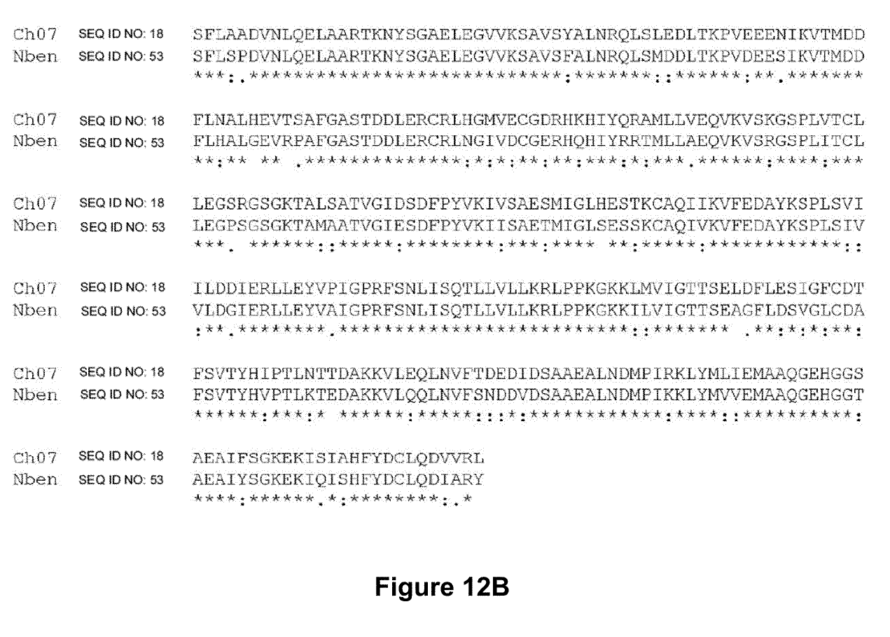

Mixed cultures of N. benthamiana NSF (NSF_{N.benth}, 81% identity to NSF_{Ch07}, see FIG. 12 for alignment) and α-SNAP_{Rhg1}LC, were agroinfiltrated as in FIG. 4A. EV, NSF_{Ch13} and NSF_{RAN07} were agroinfiltrated as controls. NSF_{Ch13} gave visible protection relative to an empty vector, while NSF_{RAN07} co-expression gave strong protection (FIG. 4B). In contrast, NSF_{N.benth} co-expression was similar to empty vector controls (FIG. 4B). Expressing soybean NSFs or NSF_{N.benth} with an empty vector at the same ratios used for complementation did not cause macroscopic phenotypes suggestive of stress (FIG. 11D).

Physical binding with Rhg1 resistance-type α-SNAPs using recombinant NSF_{N.benth} protein was determined. Whereas NSF_{N.benth} readily bound α-SNAP_{Rhg1}WT, NSF_{N.benth} binding to Rhg1 resistance-type α-SNAPs was much lower, only slightly over controls (α-SNAP lacking the C-terminus or no-α-SNAP) (FIG. 4C). This suggested a biochemical explanation for why Rhg1 resistance type α-SNAPs—but not WT α-SNAPs—provoke strong cell death responses in N. benthamiana: the endogenous N. benthamiana NSF binds WT α-SNAPs but not Rhg1 resistance type α-SNAPs.

Complementation assays using NSF_{RAN07} or NSF_{Ch07} were performed to determine if either could prevent cell-death caused by α-SNAP_{Rhg1}LC₁₋₂₇₉, which lacks the final 10 C-terminal residues and does not bind NSF_{RAN07} or NSF_{Ch07} in vitro. Neither NSF_{RAN07} nor NSF_{Ch07} prevented the cell death caused by α-SNAP_{Rhg1}LC₁₋₂₇₉ whereas either complemented the cell death induced by full length α-SNAP_{Rhg1}LC (FIG. 11E).

The impact of the penultimate α-SNAP residue implicated in NSF-ATPase stimulation was determined using complementation assays with NSF_{RAN07} or NSF_{Ch07}. Complementation of α-SNAP_{Rhg1}LC I₂₈₉A was evident, but was less than that observed for α-SNAP_{Rhg1}LC (FIG. 4D).

### Example 6: 100% of the Predicted Rhg1⁺ Soybean Accessions in the USDA Soybean Collection, and 7% of the Rhg1⁻ Soybean Accessions, Contain the SoySNP50K NSF_{RAN07} R₄Q Amino Acid Polymorphism

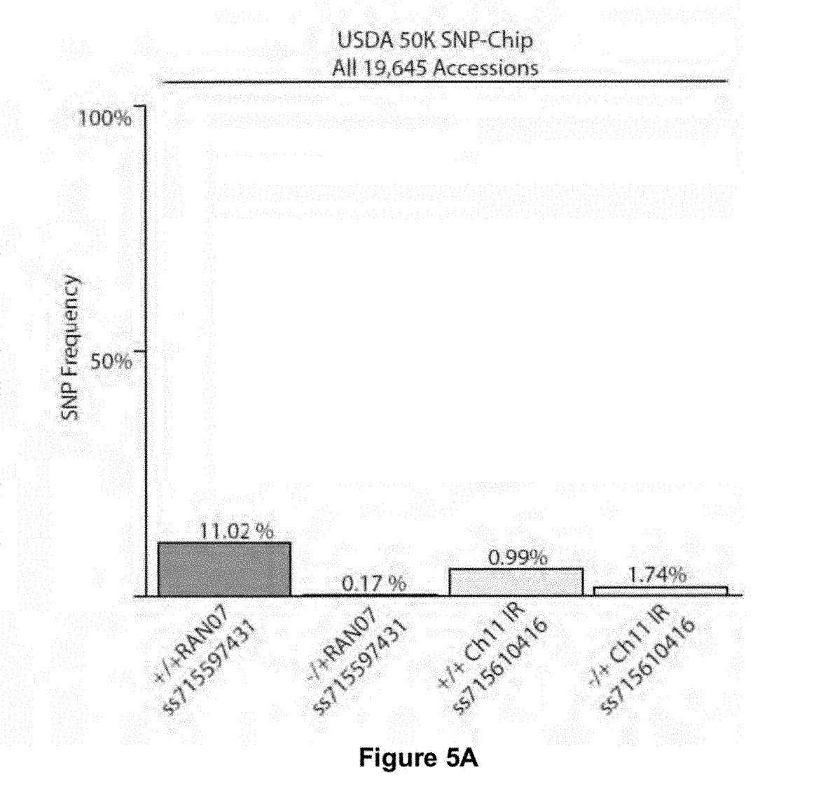

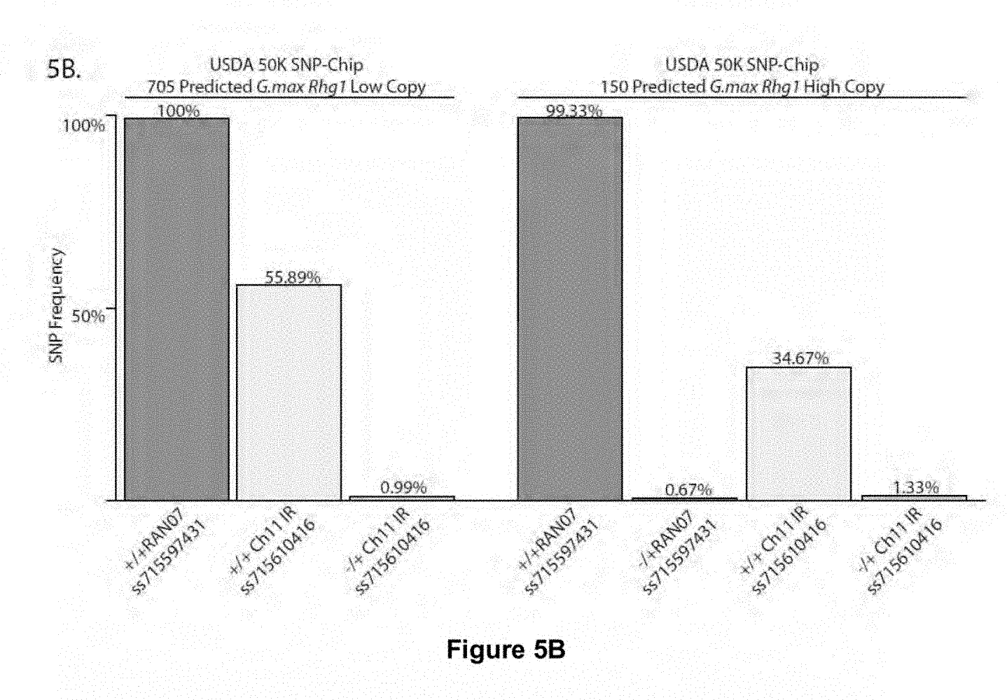

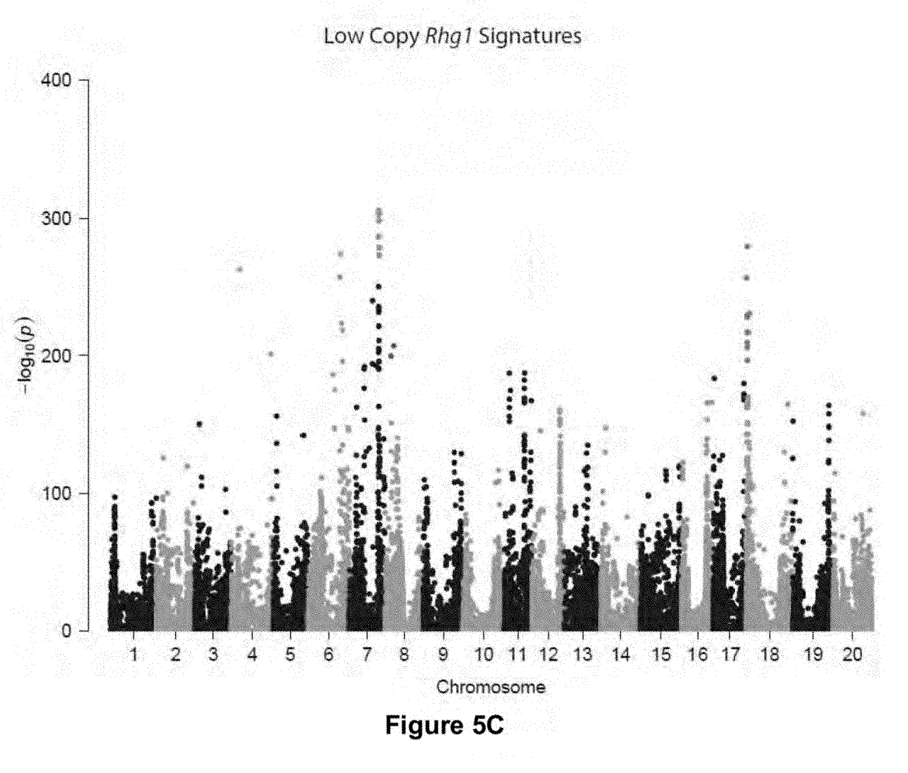

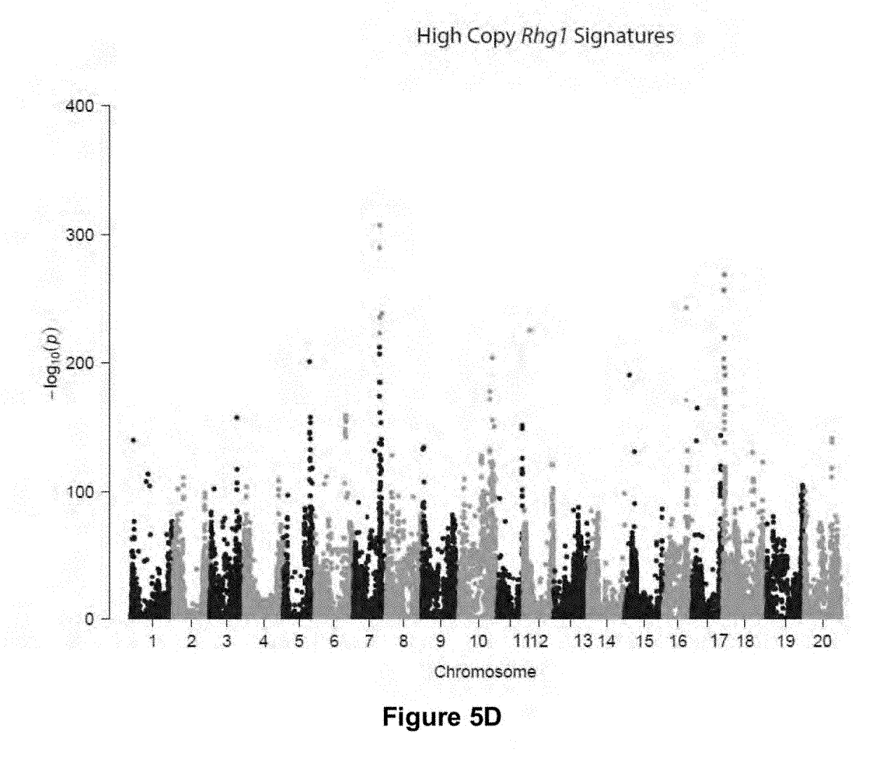

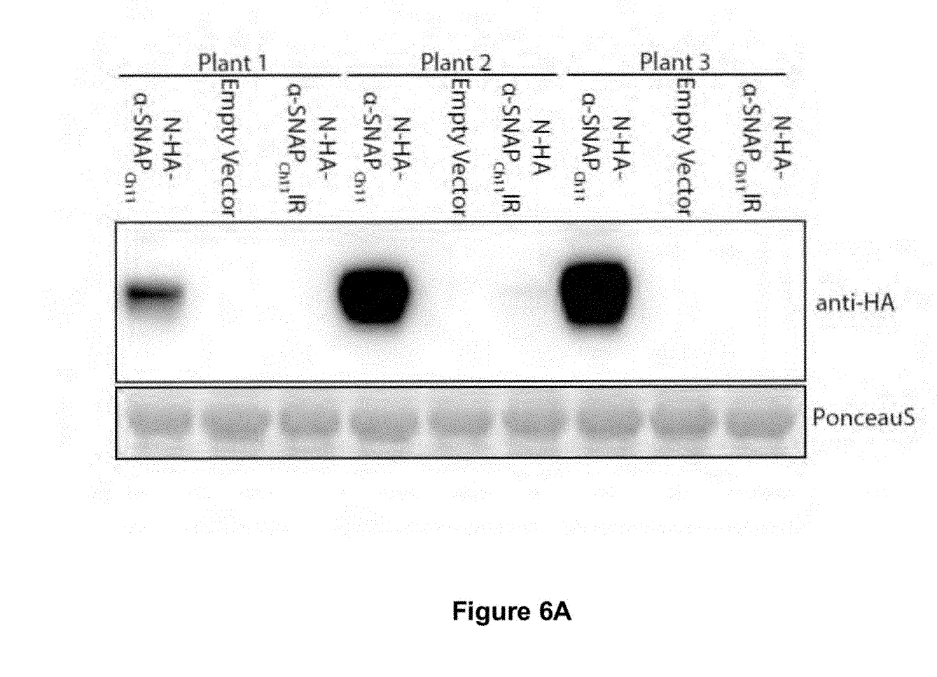

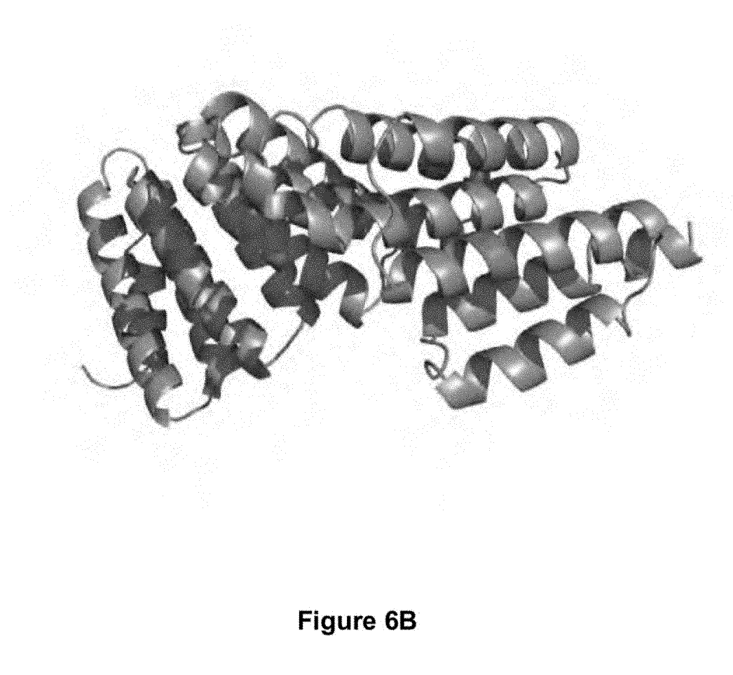

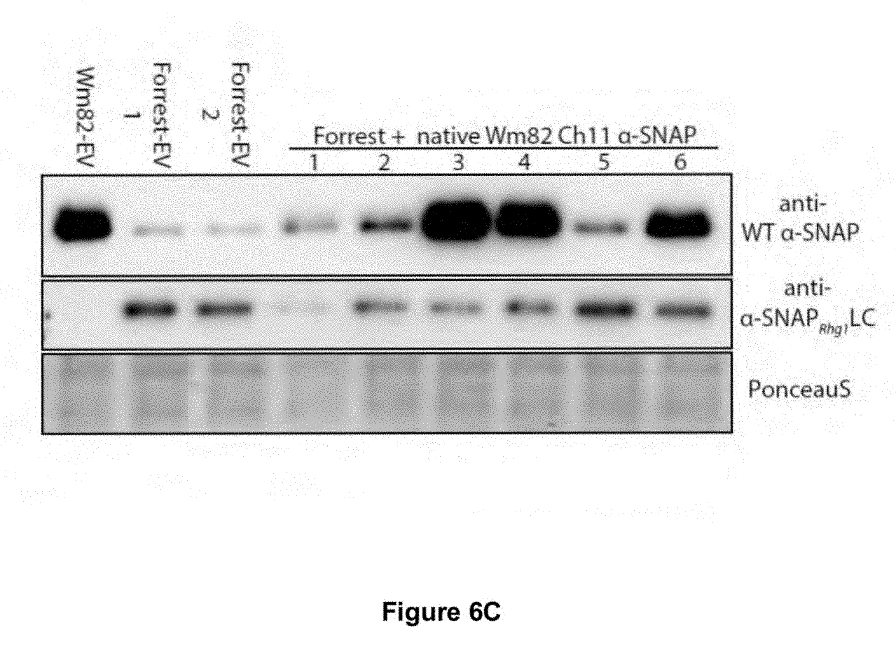

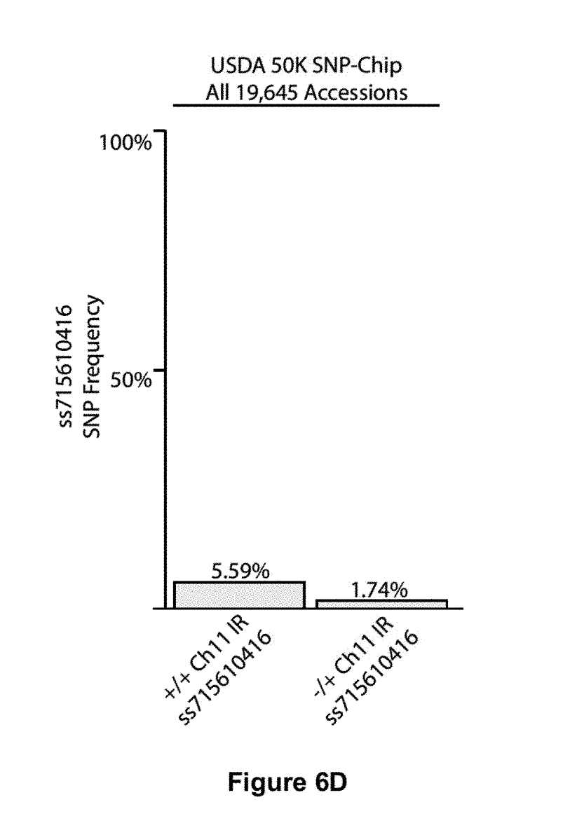

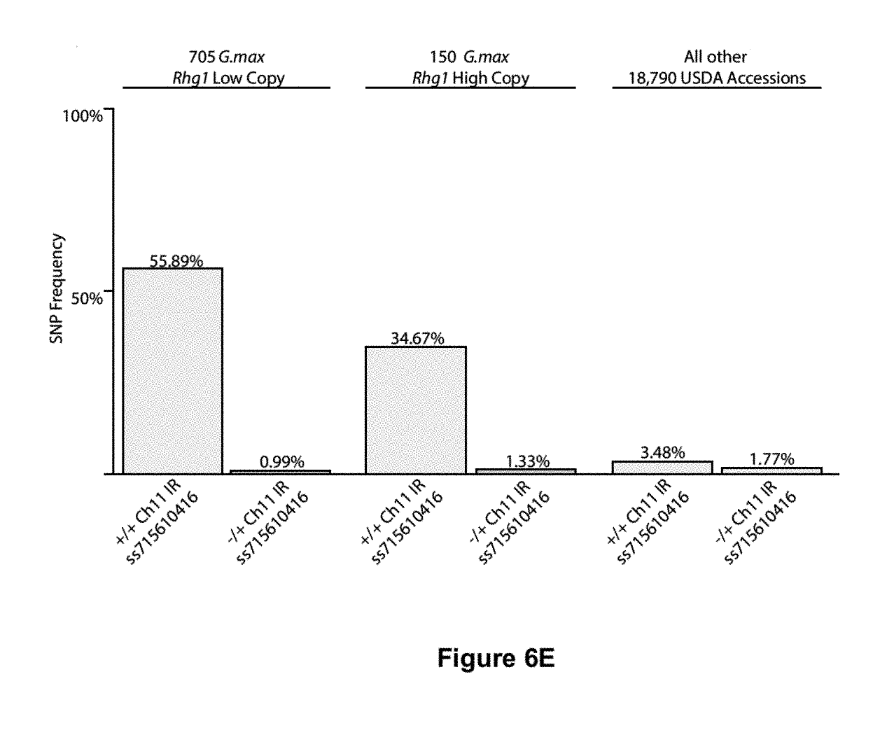

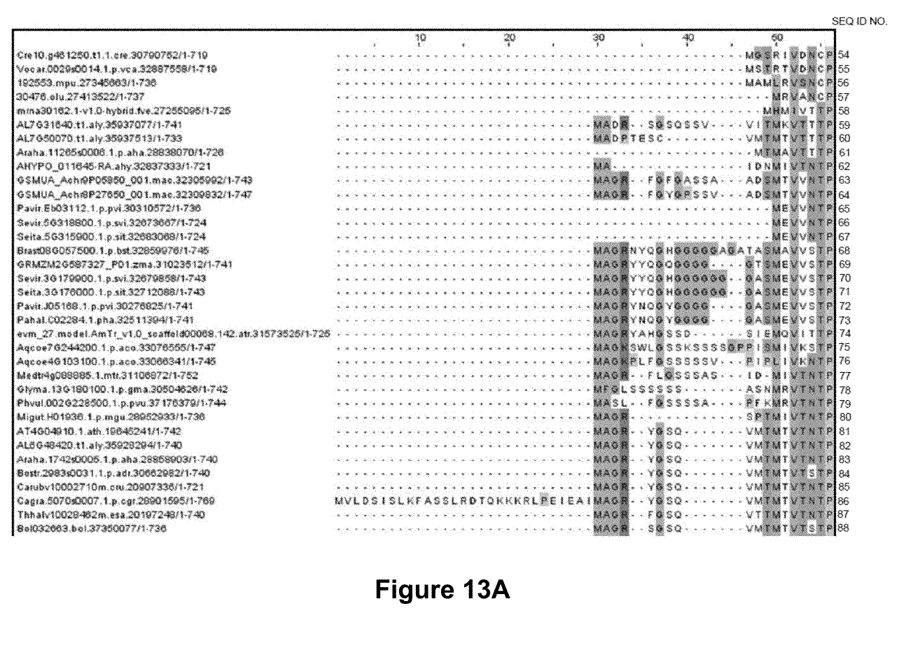

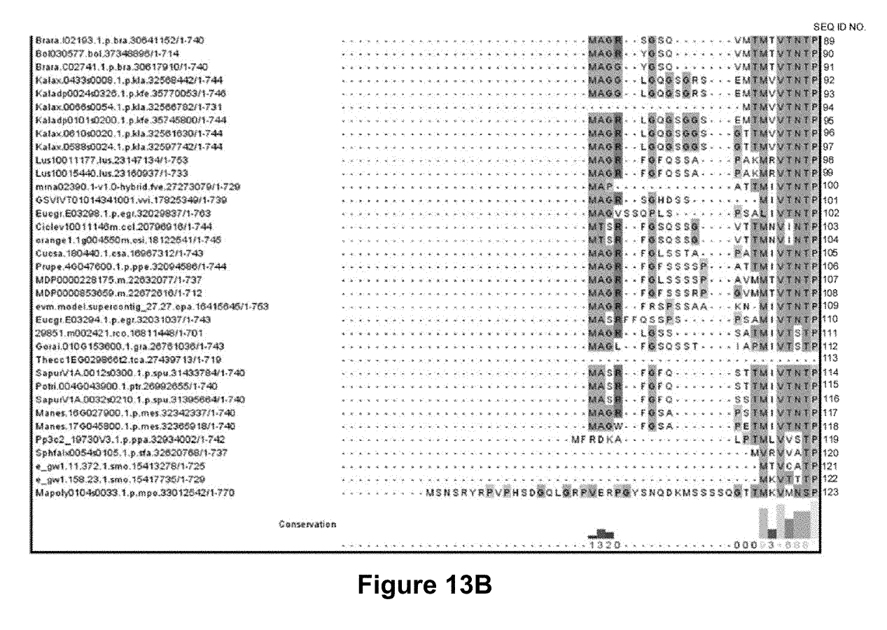

NSF_{RAN07} was present in all Rhg1-containing HG type and NAM lines, but whether this Rhg1/NSF_{RAN07} association was universal rather than “frequent” was further investigated. First, the approximate NSF_{RAN07} allele frequency was determined. In 2015, Song et al. reported genotyping the USDA soybean germplasm collection of ˜20,000 accessions—collected from over 80 countries—using a 50,000 SNP DNA microarray chip (SoySNP50K iSelect BeadChip). These data were available in a searchable SNP database at Soybase (Soybase.org/snps/) (Grant et al., 2010, Nucleic Acids Res 38, D843-846; Song et al., 2013, PLoS One 8, e54985; Song et al., 2015, PLoS Genet 11, e1005200). Using the Soybase genome browser, a C/T SNP was found to be involved using the SoySNP50K (ss715597431, Gm07:36,449,014) that causes the NSF_{RAN07} R₄Q polymorphism. Analyzing all 19,645 USDA soybean accessions for ss715597431, the NSF_{RAN07} allele frequency in the USDA collection was estimated at 11.0% (2,165+/+, 33+/−) (FIG. 5A). While NSF in most model eukaryotes contains R₄, it remained unclear whether Q₄ occurs in other plant NSFs. To determine if the NSF_{RAN07} R₄Q is unusual among plants, R₄ conservation across plant NSF sequences available on Phytozome (Goodstein et al., 2012, Nucleic Acids Res 40, D1178-D1186) was examined. Notably, Q₄ was not in the queried NSF predicted protein sequences for any other plant species (FIG. 13).

Rhg1-mediated SCN resistance is uncommon among soybean accessions and less than 5% of the USDA soybean collection carries a multi-copy Rhg1 haplotype. Previously, Lee et al. identified SoySNP50K signatures for Rhg1_{HC}, Rhg1_{LC} and single copy (SCN-susceptible) haplotypes, and estimated that 705 Rhg1_{LC} and 150 Rhg1_{HC} accessions were in the USDA Glycine max collection (Lee et al., 2015, Mol Ecol 24, 1774-1791). Using these 855 Rhg1-signature accessions, a 100% incidence of the ss715597431 NSF_{RAN07} signature was determined for multi-copy Rhg1-signature Glycine max (FIG. 5B).

If NSF_{RAN07} is needed for the survival of Rhg1-containing soybean plants, then, all Rhg1 accessions should carry NSF_{RAN07}. As such, SNPs within the locus underlying Rhg1 co-segregation should be maintained, while SNPs at neighboring loci, though tightly linked, would not be under stringent selection and hence should be less conserved. To narrow in on the Rhg1 co-segregating locus within the interval, amino acid changes within candidate loci adjacent to RAN07 from Rhg1-carrying HG and NAM lines, between markers ss715597415 and ss715597431, were examined. NSF_{RAN07} SNPs, especially those causing the 5 N-domain polymorphisms, were 100% maintained across all Rhg1-containing varieties. On the other hand, SNPs causing amino acid changes within candidate loci adjacent to NSF_{RAN07}, were not 100% conserved across all Rhg1-containing varieties, unlike NSF_{RAN07} (Table 3). The predicted amino acid sequence of most candidate loci matches Wm82 (SCN-susceptible) sequence, and among candidate loci with amino acid substitutions, only NSF_{RAN07} has the same consistent amino acid changes across all examined Rhg1-containing germplasm (Table 3). In addition to the observed biochemical and genetic complementation of Rhg1 α-SNAPs by NSF_{RAN07}, candidate gene allele frequency further implicates NSF_{RAN07} as the gene responsible for co-segregation with Rhg1.

TABLE 3
Amino acid polymorphisms of genes within the chromosome 07 interval co-segregating with Rhg1.

| | ss715597431 | | | | ss715597413 | | | ss715597410 | |
|---|---|---|---|---|---|---|---|---|---|
| | Soybean Line | | | | | | | | |
| | | Glyma | | | | | | | Glyma |
| | | 07g195800 | Glyma | Glyma | Glyma | Glyma | Glyma | Glyma | 07g195100 |
| | Glyma | Rubber | 07g195700 | 07g195600 | 07g195500 | 07g195400 | 07g195300 | 07g195200 | LRR |
| | 07g195900 | Elongation | DNA Mismatch | No annotated | TFII H | E3 Ubiquitin | Asparagine | Conserved | Containing |
| | NSF | Factor | Repair MutS2 | domains | Polypeptide 4 | Ligase | Synthase | Protein | Protein |
| PI89772 | R₄Q, N₂₁Y, | K₃N, F₁₃₇S | T₂₁A, K₂₃R, G₁₀₉C, | WT | WT | WT | WT | WT | WT |
| | S₂₅N, ∧₁₁₆F, | | H₁₁₅Q, V₃₄₅I, D₃₆₄N, | | | | | | |
| | M₁₆₁I | | M₄₀₆T, Q₈₁₈K | | | | | | |
| PI90763 | R₄Q, N₂₁Y, | K₃N, L₄₂R, | T₂₁A, K₂₃R, G₁₀₉C, | WT | WT | WT | WT | WT | WT |
| | S₂₅N, ∧₁₁₆F, | F₁₃₇S | H₁₁₅Q, V₃₄₅I, D₃₆₄N, | | | | | | |
| | M₁₈₁I | | M₄₀₆T, Q₈₁₈K | | | | | | |
| PI209332 | R₄Q, N₂₁Y, | K₃N, L₄₂R, | T₂₁A, K₂₃R, V₃₄₅I, | WT | WT | WT | WT | WT | WT |
| | S₂₅N, ∧₁₁₆F, | F₁₃₇S | D₃₆₄N, M₄₀₈T, Q₈₁₈K | | | | | | |
| | M₁₈₁I | | | | | | | | |
| CLOJO95-4-6 | R₄Q, N₂₁Y | K₃N, L₄₂R, | T₂₁A, K₂₃R, G₁₀₉C, | WT | WT | WT | WT | WT | WT |
| | S₂₅N, ∧₁₁₆F, | F₁₃₇S | H₁₁₅Q, V₃₄₅I, D₃₆₄N, | | | | | | |
| | M₁₈₁I | | M₄₀₈T, Q₈₁₈K | | | | | | |
| IA3023 | WT | L₄₂R, F₄₃₇S | D₃₆₄N, M₄₀₆T, Y₅₇₆F | WT | WT | WT | E₄₆G | D₆₀A, S₆₄P | WT |
| LD00-3309 | R₄Q, N₂₁Y | K₃N, L₄₂R, | T₂₁A, K₂₃R, G₁₀₉C, | WT | WT | WT | WT | WT | WT |
| | S₂₅N, ∧₁₁₆F, | F₁₃₇S | H₁₁₅Q, V₃₄₅I, D₃₆₄N, | | | | | | |
| | M₁₈₁I | | M₄₀₆T, G₅₁₈C, Q₈₁₈K | | | | | | |
| PI 437654 | R₄Q, N₂₁Y | K₃N, F₁₃₇S | T₂₁A, K₂₃R, G₁₀₉C, | WT | WT | WT | WT | WT | WT |
| | S₂₅N, ∧₁₁₆F, | | H₁₁₅Q, V₃₄₅I, D₃₆₄N, | | | | | | |
| | M₁₈₁I | | M₄₀₆T, Q₈₁₈K | | | | | | |
| PI548402 | R₄Q, N₂₁Y | K₃N, F₁₃₇S | T₂₁A, K₂₃R, G₁₀₉C, | WT | WT | WT | WT | WT | WT |
| | S₂₅N, ∧₁₁₆F, | | H₁₁₅Q, V₃₄₅I, D₃₆₄N, | | | | | | |
| | M₁₈₁I | | M₄₀₆T, Q₈₁₈K | | | | | | |
| Magellan | WT | L₄₂R, F₁₃₇S | D₃₆₄N, M₄₀₆T | WT | WT | WT | E₄₆G | D₆₀A, S₆₄P | WT |
| Maverick | R₄Q, N₂₁Y | K₃N, F₁₃₇S | T₂₁A, K₂₃R, G₁₀₉C, | WT | WT | WT | WT | WT | WT |
| | S₂₅N, ∧₁₁₆F, | | H₁₁₅Q, V₃₄₅I, D₃₆₄N, | | | | | | |
| | M₁₈₁I | | M₄₀₆T, Q₈₁₈K | | | | | | |
| PI548316 | R₄Q, N₂₁Y | K₃N, F₁₃₇S | T₂₁A, K₂₃R, G₁₀₉C, | WT | WT | WT | WT | WT | WT |
| | S₂₅N, ∧₁₁₆F, | | H₁₁₅Q, V₃₄₅I, D₃₆₄N, | | | | | | |
| | M₁₈₁I | | M₄₀₆T, Q₈₁₈K | | | | | | |

### Example 7: All Rhg1⁺F5-Derived Recombinant Inbred Lines (RILs) from NAM Population Crosses Also Carry NSF_{RAN07}

The NSF_{RAN07} data from the USDA soybean germplasm collection are an indication of strong segregation distortion. However, Webb et al. (1995) reported that only 91 of 96 lines with a resistant parent marker type linked to Rhg1 also had a resistant parent marker type near the NSF_{RAN07} QTL (Webb et al., 1995, Theor Appl Genet 91, 574-581). Therefore, lines with Rhg1 were investigated for inheritance of NSF_{RAN07} in the progeny of more recent biparental crosses. From the Soybean Nested Associated Mapping (SoyNAM) project (Song et al., 2017,Plant Genome 10(2)), genotypic data for populations of RILs developed from crosses of the IA3023 (SCN-susceptible) hub-parent to eight different soybean accessions carrying either Rhg1_{HC} (seven accessions) or Rhg1_{LC} (one accession) were examined. There were 122 to 139 RILs in each population and the segregation for NSF_{RAN07}:NSF_{Ch07}WT in soybean lines lacking Rhg1 did not deviate from the null hypothesis of 1:1 segregation in six of the eight populations. Across populations, there was a significant (α=0.05) deviation from a 1:1 segregation with a significantly greater number of RILs with NSF_{RAN07} than NSF_{Ch07}WT. The segregation distortion for NSF_{RAN07} was obvious among RILs that carried a resistance-associated Rhg1 allele but, out of a total of 309 Rhg1⁺RILs, 8 appeared to have possibly inherited Rhg1_{HC} or Rhg1_{LC} but not NSF_{RAN07} while the remainder had NSF_{RAN07}. This was based upon the lower-density SoySNP6K mapping data that that did not include perfect genetic markers for Rhg1 and NSF. Polymorphisms within Rhg1 and NSF_{RAN07} genes were genotyped using primers that detect the Rhg1 repeat junction and a WT NSF_{Ch07} vs. NSF_{RAN07} allele. All 8 re-examined RILs that inherited Rhg1_{HC} or Rhg1_{LC} also inherited the NSF_{RAN07} \hat{ }116 F and M₁₈₁I mutations meaning that all 309 RILs that carried the resistance associated Rhg1 also carried NSF_{RAN07} (Table 4).

TABLE 4
NSF_{RAN07} co-segregates with Rhg1 in all Rhg1-containing F_{2:5} offspring from Rhg1⁺ × rhg1⁻ crosses

| Diverse | RR (Ch07, | RS(Ch07, | SR(Ch07, | SS(Ch07, | HR(Ch07, | HS(Ch07, | HH(Ch07, | RH(Ch07, | SH(Ch07, |
|---|---|---|---|---|---|---|---|---|---|
| Parent | Ch18) | Ch18) | Ch18) | Ch18) | Ch18) | Ch18) | Ch18) | Ch18) | Ch18) |
| 4J105-3-4 | 41 | 41 | 2 | 31 | 9 | 3 | 1 | 9 | 0 |
| CL0J095-4-6 | 35 | 45 | 0 | 37 | 6 | 7 | 0 | 7 | 1 |
| LD00-3309 | 38 | 45 | 1 | 27 | 8 | 10 | 3 | 7 | 0 |
| LD01-5907 | 32 | 32 | 1 | 42 | 0 | 6 | 1 | 6 | 2 |
| LD02-4485 | 37 | 50 | 1 | 28 | 10 | 7 | 1 | 5 | 0 |
| LD02-9050 | 43 | 31 | 2 | 34 | 10 | 10 | 1 | 4 | 0 |
| Maverick | 31 | 34 | 0 | 41 | 8 | 8 | 3 | 8 | 1 |
| LG05-4292 | 44 | 41 | 1 | 30 | 1 | 3 | 0 | 7 | 0 |
| Totals | 301 | 319 | 8* | 270 | 52 | 54 | 10 | 53 | 4 |

R refers to allele from Rhg1 resistant parent.
S refers to allele from SCN-susceptible parent
Genotype order: first allele is chr 7 (RAN07 interval) and second is chr 18 (Rhg1 interval)
*All 8 re-examined RILs that inherited Rhg1 _{HC} or Rhg1 _{LC} also inherited the NSF _{RAN07} ^{∧}116 F and MI mutations meaning that all 309 RILs that carried the resistance associated Rhg1 also carried NSF _{RAN07}

### Example 8: NSF-RAN07 Aids in the Production of Transgenic Soybean Lines that Express an SCN-Resistance-Associated Rhg1 α-SNAP

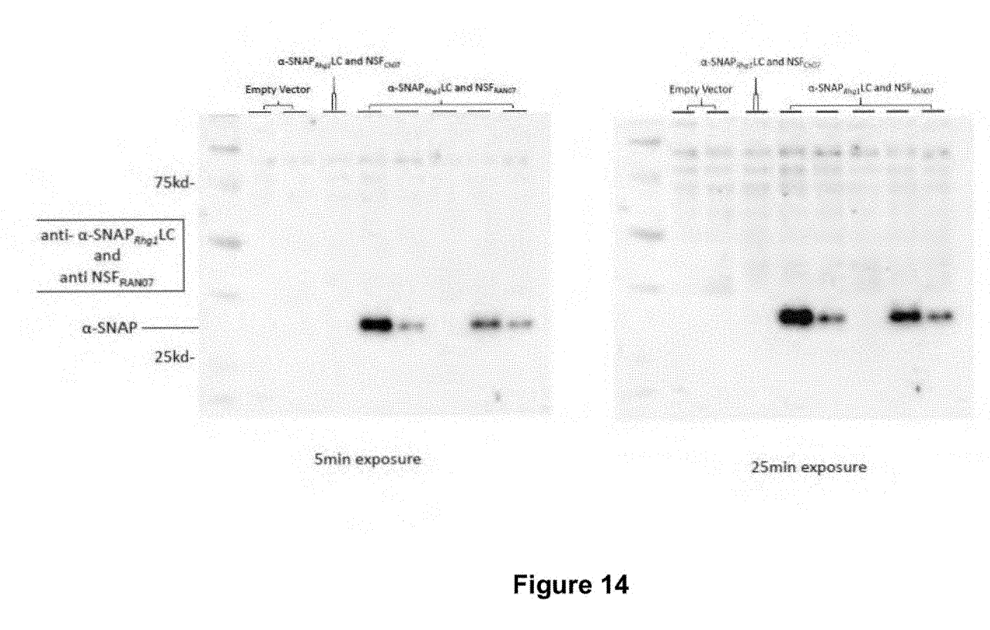

In previous work, attempts to generate transgenic soybean lines with DNA constructs derived in part from the Rhg1 locus had failed to generate lines that express α-SNAP_{Rhg1}LC or α-SNAP_{Rhg1}HC protein variants. This was despite successes within the same project in generating stably transformed transgenic soybean lines that express other genes or gene silencing constructs. That work was done using soybean variety Thorne, which does not carry an NSF_{RAN07}-encoding allele of Glyma.07G195900. In subsequent collaborative work with the University of Wisconsin—Madison Wis. Crop Innovation Center (Middleton, Wis.), an experiment was initiated in which soybean variety Williams 82 was transformed with DNA constructs designed to express α-SNAP_{Rhg1}LC or α-SNAP_{Rhg1}WT protein, together with either NSF_{RAN07} or NSF_{Ch07}WT protein, or no added NSF protein. Williams 82 lacks NSF_{RAN07} and lacks resistance-associated Rhg1. The respective DNA constructs, which used a Glycine max ubiquitin promoter sequence to drive expression of Glyma.18G022500 protein coding sequences, or Glyma.07G195900 and Glyma.18G022500 protein coding sequences on the same plasmid, were built into plasmid pC23S, a binary plasmid conferring spectinomycin resistance. Similar numbers of Williams 82 embryos were treated with the respective Agrobacterium tumefaciens strain for each DNA construct (approximately 300 embryos per Agrobacterium strain). After co-culture of the embryos with the designated Agrobacterium strain, counter-selection against the Agrobacterium was applied, and embryos were then grown on growth media containing spectinomycin. Embryos that were able to grow successfully on spectinomycin were transferred to new spectinomycin selection media, and plantlets producing new leaves and roots were then transferred to the greenhouse and grown for seed production. If the DNA used for plant transformation was phenotypically neutral, similar numbers of Williams 82 transformants would be expected for each DNA construct if using the same plasmid vector and processing all of the transformants similarly within the same experiment. However, there was a notable lack of recovery of spectinomycin-resistant transformants for soybean lines that received a DNA construct encoding α-SNAP_{Rhg1}LC expression. Zero lines were recovered for expression of only α-SNAP_{Rhg1}LC, and only one line was recovered for expression of α-SNAP_{Rhg1}LC+NSF_{Ch07}WT (Table 5). Immunoblot testing for presence of α-SNAP_{Rhg1}LC protein revealed that the one transgenic line for the α-SNAP_{Rhg1}LC+NSF_{Ch07}WT DNA construct failed to express α-SNAP_{Rhg1}LC protein (FIG. 14). In contrast, four of the five lines that received the α-SNAP_{Rhg1}LC+NSF_{RAN07}WT DNA construct did express α-SNAP_{Rhg1}LC protein (FIG. 14). These findings provide further evidence that presence of a nematode resistance-associated Rhg1 α-SNAP protein is poorly tolerated in soybean lines that express only wild-type NSF proteins, and that NSF_{RAN07}WT or a similarly suitable NSF partner protein is necessary to recover viable soybean lines that express a nematode resistance-associated Rhg1 α-SNAP.

TABLE 5
Recovery rate of transgenic soybean lines expressing
SCN-resistance-associated Rhg1 α-SNAP

| DNA construct used to | Number of |
|---|---|
| transform soybean | Williams 82 |
| variety Williams 82 | transformants recovered |
| pC23S (empty vector) | 11 |
| α-SNAP-WT (no added NSF) | 5 |
| α-SNAP-Rhg1-LC (no added | 0 |
| NSF) | |
| NSF-WT + α-SNAP-WT | 3 |
| NSF-RAN07 + α-SNAP-WT | 2 |
| NSF-WT + α-SNAP-Rhg1-LC | 1 |
| NSF-RAN07 + α-SNAP-Rhg1-LC | 5 |

### Example 9: Modified NSF BLASTp Alignment in Plant Species

The WT NSF sequence for wild type Glycine max (accession number AWH66430.1 was entered into BLASTp and modified at R4Q, N21Y, S25N, (del)116F, and M1811. The modified sequence was then entered into BLASTp to determine the occurrence, in the NSF proteins of 100 other plant species, of amino acids at the protein residue positions of the above key NSF_{RAN07} amino acids. The amino acid expressed at positions 4, 21, 25, 116 and 181 in the BLASTp results were compared against the Glycine max NSF_{RAN07} and the data entered into Table 6. In sequences for which BLASTp protein alignment started after the designated amino acid position, that position is marked N/A. Naturally occurring proteins encoding the R4Q or N21Y residues found in Glycine max NSF_{RAN07} were not present in the sequences for any of the other plant species compared via BLASTp.

TABLE 6
Modified NSF BLASTp Alignment in Plant Species

| | | | | | | | | | | % |
|---|---|---|---|---|---|---|---|---|---|---|
| | | NSF Accession | % | | | | | — | M181I | Query |
| Genus Species | Plant | Number | Identity | Identities | R4Q | N21Y | S25N | 116F | (Subst) | Cover |
| Glycine Max | Soybean | XP_003529321.1 | 99.33 | 742/747 | R | N | S | — | M | 99.73 |
| Predicted | | | | | | | | | | |
| Glycine Max | Soybean | XP_003541535.1 | 97.57 | 724/742 | N/A | N | S | — | M | 98.65 |
| Predicted | | | | | | | | | | |
| Glycine Soja | Wild Soybean | KHN10009.1 | 95.98 | 717/747 | N/A | N | S | — | M | 97.19 |
| Phaseolus | Common/ | XP_007159324.1 | 92.50 | 691/747 | L | N | T | — | L | 96.79 |
| Vulgaris | Green Bean | | | | | | | | | |
| Glycine Max | Hypo Glycine | KRH50034.1 | 99.00 | 696/703 | R | N | S | — | M | 99.57 |
| | Max | | | | | | | | | |
| Vigna Radiata var. | Mung Bean | XP_014510227.1 | 92.60 | 688/743 | N/A | N | S | — | M | 96.77 |
| radiata | | | | | | | | | | |
| Vigna angularis | Adzuki Bean | XP_017411260.1 | 92.60 | 688/743 | N/A | N | S | — | M | 96.64 |
| Arachis ipaensis | Peanut | XP_016190089.1 | 89.83 | 671/747 | L | N | Q | — | M | 94.78 |
| Arachis | Wild Peanut | XP_015956468.1 | 89.83 | 671/747 | L | N | Q | — | M | 94.91 |
| duranensis | | | | | | | | | | |
| Lupinus | Lupin | XP_019445668.1 | 88.89 | 664/747 | W | N | Q | — | M | 94.78 |
| angustifolius | | | | | | | | | | |
| Lupinus | Narrow leaf | XP_019421896.1 | 90.29 | 660/731 | N/A | N | Q | — | M | 95.21 |
| angustifolius | Lupiin (Herb) | | | | | | | | | |
| Cajanus cajan | Pigeon Pea | XP_020225776.1 | 90.85 | 665/732 | N/A | N | Q | — | M | 95.36 |
| | (Legume) | | | | | | | | | |
| Cajanus cajan | Pigeon Pea | KYP76270.1 | 90.45 | 663/733 | N/A | N | Q | — | M | 95.23 |
| | (Legume) | | | | | | | | | |
| Vigna angularis | Adzuki Bean | KOM31050.1 | 88.69 | 659/743 | N/A | N | S | — | M | 92.6 |
| Medicago | BarrelClover | XP_024637282.1 | 85.41 | 638/747 | R | N | Q | I | M | 93.04 |
| truncatula | (small | | | | | | | | | |
| | Mediterranean | | | | | | | | | |
| | Legume) | | | | | | | | | |
| cephalotus | Australian | GAV67671.1 | 84.74 | 633/747 | R | N | A | — | I | 92.37 |
| follicularis | Pitcher Plant | | | | | | | | | |
| Quercus suber | Cork Oak | XP_023924241.1 | 86.44 | 631/730 | N/A | N | A | — | M | 95.07 |
| Citrus clementina | Clementine | XP_006428558.1 | 84.739 | 633/747 | R | N | A | — | I | 93.57 |
| Medicago | BarrelClover | KEH31080.1 | 84.707 | 637/752 | R | N | Q | I | M | 92.29 |
| truncatula] | (small | | | | | | | | | |
| | Mediterrian | | | | | | | | | |
| | Legume) | | | | | | | | | |
| Cicer arietinum | ChickPea | XP_004505051.1 | 88.615 | 646/729 | N/A | N | T | I | M | 95.2 |
| Citrus sinensis | Sweet | KDO54905.1 | 84.626 | 633/748 | R | N | A | — | I | 93.45 |
| | Oranges | | | | | | | | | |
| | (blood, navel) | | | | | | | | | |
| Populus | Black | XP_002305796.2 | 84.605 | 632/747 | R | N | A | — | M | 91.97 |
| trichocarpa | cottonwood | | | | | | | | | |
| Herrania | Colombian | XP_021294427.1 | 84.584 | 631/746 | R | S | T | — | M | 92.63 |
| umbratica | Cocoa | | | | | | | | | |
| Populus | Desert Poplar | XP_011027829.1 | 84.605 | 632/747 | R | N | A | — | M | 91.83 |
| euphratica | | | | | | | | | | |
| Jatropha curcas | Jatropha | XP_012091606.1 | 85.346 | 629/737 | N/A | N | P | — | M | 93.22 |
| | curcas | | | | | | | | | |
| Ziziphus jujuba | Jujube red | XP_015890094.1 | 85.121 | 635/746 | R | N | P | — | M | 92.63 |
| | date | | | | | | | | | |
| Durio zibethinus | Durio | XP_022720468.1 | 84.471 | 631/747 | R | G | T | — | M | 92.5 |
| | zibethinus | | | | | | | | | |
| Manihot esculenta | Yuca | XP_021597323.1 | 84.987 | 634/746 | R | N | A | — | M | 91.69 |
| Pyrus × | Chinese white | XP_009339728.1 | 85.007 | 635/747 | R | N | P | — | M | 93.17 |
| bretschneideri | pear | | | | | | | | | |
| Morus notabilis | Black | XP_024029108.1 | 84.718 | 632/746 | R | S | T | — | M | 93.16 |
| | Mulberry | | | | | | | | | |
| Gossypium | Cotton Plant | XP_012450449.1 | 84.07 | 628/747 | L | S | T | — | M | 92.37 |
| raimondii | | | | | | | | | | |
| Citrus unshiu | Mandarin | GAY44590.1 | 84.337 | 630/747 | R | N | A | — | I | 92.9 |
| | Orange | | | | | | | | | |
| Quercus suber | Cork Oak | XP_023927780.1 | 85.753 | 626/730 | T | N | A | — | M | 94.38 |
| Malus domestica | Apple Tree | XP_008364158.1 | 84.873 | 634/747 | R | N | P | — | M | 93.04 |
| Gossypium | Cotton Tree | XP_017642474.1 | 84.853 | 633/7465 | W | S | P | — | M | 91.82 |
| arboreum | | | | | | | | | | |
| Gossypium | Cotton Tree | XP_017646058.1 | 83.668 | 625/747 | L | S | T | — | M | 92.1 |
| arboreum | | | | | | | | | | |
| Gossypium | Mexican | XP_016676150.1 | 83.534 | 624/747 | L | S | T | — | M | 92.1 |
| hirsutum | Cotton Tree | | | | | | | | | |
| Hevea brasiliensis | Rubberwood | XP_021641739.1 | 84.584 | 631/746 | R | N | S | — | M | 91.42 |
| Durio zibethinus | Durian Tree | XP_022724072.1 | 84.048 | 686/746 | R | S | T | — | M | 91.96 |
| Lupinus | Lupin | OIV94352.1 | 91.215 | 623/683 | N/A | N/A | N/A | — | M | 96.34 |
| angustifolius | | | | | | | | | | |
| Gossypium | Mexican | XP_016683459.1 | 83.802 | 626/747 | L | S | T | — | M | 91.97 |
| hirsutum | Cotton Tree | | | | | | | | | |
| Gossypium | Cotton | XP_012450761.1 | 84.048 | 627/746 | W | S | P | — | M | 91.96 |
| raimondii | | | | | | | | | | |
| Gossypium | Cotton | KJB68632.1 | 83.936 | 627/747 | W | S | P | — | M | 91.83 |
| raimondii | | | | | | | | | | |
| Prunus avium | Sweet/wild | XP_021825850.1 | 83.78 | 625/746 | R | N | A | — | M | 92.76 |
| | Cherry tree | | | | | | | | | |
| Hevea brasiliensis | Rubberwood | XP_021640046.1 | 83.512 | 623/746 | R | N | L | — | M | 91.69 |
| Lupinus | Blue Lupine | OIW10410.1 | 85.007 | 635/747 | W | N | Q | — | M | 90.36 |
| angustifolius | | | | | | | | | | |
| Gossypium | Cotton Plant | XP_012450763.1 | 84.048 | 686/746 | W | S | P | — | M | 91.96 |
| raimondii | | | | | | | | | | |
| Theobroma cacao | Cacao Tree | XP_007025619.2 | 83.78 | 625/746 | R | S | A | — | M | 92.23 |
| Populus | Black | XP_006377363.1 | 83.936 | 627/747 | R | N | A | — | M | 91.43 |
| trichocarpa | cottonwood | | | | | | | | | |
| Gossypim | Cotton Plant | XP_012450762.1 | 84.048 | 627/746 | W | S | P | — | M | 91.96 |
| raimondii | | | | | | | | | | |
| Hevea brasiliensis | Rubber Tree | XP_021657769.1 | 84.316 | 629/746 | R | N | D | — | M | 91.15 |
| Eucalyptus grandis | Eucalyptus or | XP_010057417.1 | 83.914 | 626/746 | R | N | A | — | K | 92.36 |
| | Rose Gum | | | | | | | | | |
| Populus | Black | PNT11917.1 | 83.936 | 627/747 | R | N | A | — | M | 91.43 |
| trichocarpa | Cottonwood | | | | | | | | | |
| Prunus persica | Peach | XP_007214647.1 | 83.78 | 691/746 | R | N | A | — | M | 92.63 |
| Prunus mume | Japanese | XP_008225100.1 | 83.646 | 624/746 | R | N | A | — | M | 92.76 |
| | Apricot | | | | | | | | | |
| Pyrus × | Chinese white | XP_009352914.1 | 83.802 | 626/747 | R | N | A | — | M | 92.77 |
| bretschneideri | pear | | | | | | | | | |
| Hevea brasiliensis | Rubber Tree | XP_021640045.1 | 83.378 | 622/746 | R | N | L | — | M | 91.55 |
| Gossypium | Mexican | XP_016751989.1 | 83.668 | 625/747 | W | S | P | — | M | 91.7 |
| hirsutum | Cotton | | | | | | | | | |
| Gossypium | Extra long | PPS13789.1 | 83.202 | 634/762 | W | S | P | — | M | 89.76 |
| barbadense | staple cotton | | | | | | | | | |
| | (Sea Island | | | | | | | | | |
| | Cotton) | | | | | | | | | |
| Gossypium | Upland Cotton | XP_016751992.1 | 83.78 | 625/746 | W | S | P | — | M | 91.82 |
| hirsutum | | | | | | | | | | |
| Theobroma cacao | Cacao tree | XP_017978707.1 | 83.556 | 625/746 | R | S | A | — | M | 91.98 |
| Gossypium | Upland Cotton | XP_016751991.1 | 83.78 | 625/746 | W | S | P | — | M | 91.82 |
| hirsutum | | | | | | | | | | |
| Gossypium | Upland Cotton | XP_016751990.1 | 83.78 | 625/746 | W | S | P | — | M | 91.82 |
| hirsutum | | | | | | | | | | |
| Tarenaya | Spider Flower | XP_010529424.1 | 83.133 | 621/747 | R | N | A | — | M | 92.1 |
| hassleriana | | | | | | | | | | |
| Juglans regia | Walnut Tree | XP_018860049.1 | 84.146 | 621/738 | N/A | S | P | — | M | 92.95 |
| Populus | Desert Poplar | XP_011043386.1 | 83.534 | 624/747 | R | N | A | — | M | 90.9 |
| euphratica | | | | | | | | | | |
| Prunus yedoensis | King Cherry | PQM34143.1 | 83.512 | 623/746 | R | N | A | — | M | 92.09 |
| var. nudiflora | (Korean | | | | | | | | | |
| | Cherry) | | | | | | | | | |
| Carica papaya | Papaya | XP_021902227.1 | 84.182 | 628/746 | R | N | S | — | M | 92.36 |
| Cucumis melo | Muskmelon | XP_008463616.1 | 82.038 | 612/746 | R | N | Q | — | M | 92.23 |
| Manihot esculenta | Yuca | XP_021598339.1 | 83.244 | 680/746 | W | N | A | — | M | 91.15 |
| Populus | Black | PNT11918.1 | 82.827 | 627/757 | R | N | A | — | M | 90.22 |
| trichocarpa | cottonwood | | | | | | | | | |
| Gossypium | Extra long | PPD95675.1 | 82.26 | 626/761 | W | S | P | — | M | 90.01 |
| barbadense | staple cotton | | | | | | | | | |
| | (Sea Island | | | | | | | | | |
| | Cotton) | | | | | | | | | |
| Cucurbita pepo | Winter Squash | XP_023519438.1 | 81.66 | 610/747 | L | S | A | — | M | 91.7 |
| subsp. Pepo | | | | | | | | | | |
| Tarenaya | Spider Flower | XP_010538665.1 | 82.597 | 617/747 | R | N | A | — | M | 91.43 |
| hassleriana | | | | | | | | | | |
| Cucurbita | Pumpkin | XP_022927355.1 | 81.769 | 610/746 | L | S | A | — | M | 91.69 |
| moschata | | | | | | | | | | |
| Cucumis sativus | Cucumber | XP_004139535.1 | 81.769 | 610/746 | R | N | Q | — | M | 92.09 |
| Cucurbita maxima | Squash | XP_023001327.1 | 81.769 | 610/746 | L | S | A | — | M | 91.69 |
| Trifolium | Clover | GAU38492.1 | 82.097 | 642/782 | R | N | Q | I | M | 88.75 |
| subterraneum | | | | | | | | | | |
| Nicotiana tabacum | Cultivated | BAA13101.1 | 81.233 | 606/746 | R | Y | K | — | M | 91.96 |
| Tobacco | | | | | | | | | | |
| Vitis vinifera | Grape Vine | XP_002284987.1 | 82.568 | 611/740 | R | N | R | — | I | 92.03 |
| Nicotiana | Tobacco Plant | XP_009626763.1 | 81.233 | 606/746 | R | N | K | — | M | 91.96 |
| tomentosiformis | | | | | | | | | | |
| Theobroma cacao | Cacao Tree | EOY28241.1 | 85.278 | 614/720 | N/A | N/A | N/A | — | M | 93.06 |
| Sesamum indicum | Seasame | XP_011098317.1 | 82.763 | 605/731 | N/A | N | K | — | I | 91.93 |
| Malus domestica | Apple | XP_008383736.1 | 83.802 | 626/747 | R | N | A | — | M | 92.64 |
| Nicotiana | Coyote | XP_019251692.1 | 80.965 | 604/746 | R | N | K | — | M | 91.69 |
| attenuata | Tobacco | | | | | | | | | |
| Actinidia chinensis | Kiwifruit | PSR95688.1 | 81.511 | 604/741 | N/A | N | K | — | I | 91.36 |
| var. chinensis | | | | | | | | | | |
| Punica granatum | Pomegranate | PKI69442.1 | 83.469 | 616/738 | N/A | N | A | — | M | 92.14 |
| Capsicum annuum | Chili Peppers | XP_016574871.1 | 80.697 | 602/746 | R | N | K | — | M | 91.82 |
| Ipomoea nil | Morning Glory | XP_019187191.1 | 81.905 | 602/735 | N/A | N | K | — | L | 91.97 |
| Handroanthus | Pink Trumpet | PIN22741.1 | 82.538 | 605/733 | N/A | N | K | — | M | 92.22 |
| impetiginosus | Tree | | | | | | | | | |
| Vitis vinifera | Grape Vine | CBI20305.3 | 82.027 | 607/740 | N/A | N | R | — | I | 91.62 |
| Daucus carota | Carrot | XP_017252931.1 | 83.083 | 609/733 | N/A | S | K | — | M | 91.68 |
| subsp. Sativus | | | | | | | | | | |
| Solanum Pennellii | Tomato | XP_015062393.1 | 83.083 | 599/746 | R | N | K | — | M | 91.82 |
| Solanum | Potato | XP_006351809.1 | 80.295 | 599/746 | R | N | K | — | M | 91.69 |
| tuberosum | | | | | | | | | | |
| Solanum | Tomato | XP_004230528.1 | 80.295 | 598/746 | R | N | K | — | M | 91.96 |
| lycopersicum | | | | | | | | | | |
| Helianthus annuus | Sunflower | XP_022013369.1 | 81.351 | 607/740 | N/A | N | K | — | M | 91.35 |
| Gossypium | Cotton Plant | KJB66715.1 | 81.928 | 612/747 | L | S | T | — | M | 89.69 |
| raimondii (Hypo) | | | | | | | | | | |
| Macleaya cordata | Plume Poppy | OVA14922.1 | 81.325 | 614/755 | | N | S | — | M | 89.4 |

### Example 10: Modified α-SNAP BLASTp Alignment in Plant Species

The Rhg1 LC haplotype Glyma.18G022500 encoded protein sequence was entered into BLASTp and the results for 100 plant species were further examined. The BLASTp results at the α-SNAP C-terminus amino acid residues of interest (amino acid positions 208, 284, 285, 286, and 287, in the soybean Glyma.18G022500 product) were compared against the Rhg1 LC haplotype and entered into Table 7. The majority of plant species alignments terminated prior to the sequences of interest and are represented in the table as N/A.

TABLE 7
Modified α-SNAP BLASTp Alignment in Plant Species

| | | α-SNAP | | | | | | | | % |
|---|---|---|---|---|---|---|---|---|---|---|
| Genus | | Accession | % | | | | | | | Query |
| Species | Plant | Number | Identity | Identities | D208E | E284 | E285 | D286 | D287 | Cover |
| Glycine Max | Soybean | NP_001242059.2 | 100 | 289/289 | D | E | E | D | D | 100 |
| Predicted | | | | | | | | | | |
| Glycine Max | Soybean | ACU19524.1 | 99.308 | 287/289 | D | E | E | D | D | 99.65 |
| Predicted | | | | | | | | | | |
| Glycine Max | Soybean | ARD05064.1 | 99.649 | 284/285 | E | E | E | N/A | N/A | 100 |
| Predicted | | | | | | | | | | |
| Glycine Max | Soybean | ACU18668.1 | 99.298 | 283/285 | D | E | Q | N/A | N/A | 100 |
| Predicted | | | | | | | | | | |
| Glycine Max | Soybean | NP_001344346.1 | 97.578 | 282/289 | D | E | E | D | D | 98.96 |
| Predicted | | | | | | | | | | |
| Cajanus | Pigeon Pea | XP_020237258.1 | 95.848 | 277/289 | D | E | E | D | D | 97.92 |
| cajan | (Legume) | | | | | | | | | |
| Trifolium | Clover | GAU29434.1 | 91.003 | 263/289 | D | E | E | D | D | 96.89 |
| subterraneum | | | | | | | | | | |
| Medicago | Barrel Clover | XP_003601014.1 | 89.619 | 259/289 | D | E | E | D | D | 96.19 |
| truncatula | (small | | | | | | | | | |
| | Mediterranean | | | | | | | | | |
| | Legume) | | | | | | | | | |
| Quercus | Cork Oak | XP_023896842.1 | 89.273 | 258/289 | D | E | E | D | D | 96.89 |
| suber | | | | | | | | | | |
| Durio | Durio | XP_022774310.1 | 88.581 | 256/289 | D | E | E | D | D | 95.16 |
| zibethinus | zibethinus | | | | | | | | | |
| Lupinus | Lupin | XP_019456553.1 | 88.235 | 255/289 | D | E | E | D | D | 96.54 |
| angustifolius | | | | | | | | | | |
| Phaseolus | Common/ | XP_007163598.1 | 94.464 | 273/289 | D | E | E | D | D | 97.58 |
| vulgaris | Green Bean | | | | | | | | | |
| Glycine Max | Soybean | KRH14886.1 | 87.889 | 254/289 | D | E | E | D | D | 96.89 |
| Predicted | | | | | | | | | | |
| Vigna | Adzuki | XP_017407790.1 | 93.772 | 271/289 | D | E | E | D | D | 97.23 |
| angularis | Bean | | | | | | | | | |
| Cajanus | Pigeon Pea | XP_020237651.1 | 87.543 | 253/289 | D | E | E | D | D | 96.54 |
| cajan | (Legume) | | | | | | | | | |
| Juglans | Walnut Tree | XP_018821859.1 | 88.235 | 255/289 | D | E | E | D | D | 95.85 |
| regia | | | | | | | | | | |
| Vigna | Mung Bean | XP_014490390.1 | 94.118 | 272/289 | D | E | E | D | D | 96.89 |
| radiata var. | | | | | | | | | | |
| radiata | | | | | | | | | | |
| Medicago | BarrelClover | XP_003616738.1 | 86.505 | 250/289 | D | E | E | D | D | 96.19 |
| truncatula | (small | | | | | | | | | |
| | Mediterranean | | | | | | | | | |
| | Legume) | | | | | | | | | |
| Theobroma | Cacao Tree | EOY02634.1 | 88.235 | 255/289 | D | E | E | D | D | 95.5 |
| cacao | | | | | | | | | | |
| Herrania | Colombian | XP_021299224.1 | 87.889 | 254/289 | D | E | E | D | D | 95.5 |
| umbratica | Cocoa | | | | | | | | | |
| Theobroma | Cacao Tree | XP_007031708.2 | 87.889 | 254/289 | D | E | E | D | D | 95.5 |
| cacao | | | | | | | | | | |
| Cicer | ChickPea | XP_004500538.1 | 92.042 | 266/289 | D | E | E | D | D | 96.89 |
| arietinum | | | | | | | | | | |
| Phaseolus | Common/ | XP _007141718.1 | 86.159 | 249/289 | D | E | E | D | D | 96.19 |
| vulgaris | Green | | | | | | | | | |
| | Bean | | | | | | | | | |
| Phaseolus | Common/ | AHA84269.1 | 93.38 | 268/287 | D | E | E | D | E | 96.86 |
| vulgaris | Green Bean | | | | | | | | | |
| Vigna | Adzuki | XP_017429402.1 | 84.775 | 277/289 | D | E | E | D | D | 95.85 |
| angularis | Bean | | | | | | | | | |
| Lotus | Trefoil/Wild | AFK46359.1 | 91.696 | 265/289 | D | E | E | D | D | 97.23 |
| japonicus | Legume | | | | | | | | | |
| Juglans | Walnut Tree | XP_018838975.1 | 90.311 | 261/289 | D | E | E | D | D | 97.58 |
| regia | | | | | | | | | | |
| Vigna | Mung Bean | XP_014504530.1 | 84.775 | 245/289 | D | E | E | D | D | 95.85 |
| radiata var. | | | | | | | | | | |
| radiata | | | | | | | | | | |
| Gossypium | Cotton Plant | XP_012453802.1 | 85.467 | 247/289 | D | E | E | D | D | 95.85 |
| raimondii | | | | | | | | | | |
| Gossypium | Mexican | NP_001314193.1 | 86.851 | 251/289 | D | E | E | D | D | 95.16 |
| hirsutum | Cotton Tree | | | | | | | | | |
| Glycine Max | Soybean | XP_003519412.1 | 85.813 | 248/289 | D | E | E | D | D | 94.81 |
| Predicted | | | | | | | | | | |
| Arachis | Peanut | XP_016180830.1 | 91.003 | 263/289 | D | E | E | D | D | 95.85 |
| ipaensis | | | | | | | | | | |
| Gossypium | Cotton Plant | XP_012445339.1 | 86.505 | 250/289 | D | E | E | D | D | 94.81 |
| raimondii | | | | | | | | | | |
| Glycine Max | Soybean | NP_001242555.1 | 85.813 | 248/289 | G | E | G | D | D | 94.81 |
| Predicted | | | | | | | | | | |
| Lupinus | Lupin | XP_019437582.1 | 90.311 | 261/289 | D | E | E | D | D | 95.85 |
| angustifolius | | | | | | | | | | |
| Lupinus | Lupin | XP_019415244.1 | 90.657 | 262/289 | D | E | E | D | D | 95.16 |
| angustifolius | | | | | | | | | | |
| Gossypium | Mexican | XP_016677490.1 | 84.775 | 245/289 | D | E | E | D | D | 95.5 |
| hirsutum | Cotton Tree | | | | | | | | | |
| Manihot | Yuca | XP_021617295.1 | 85.121 | 246/289 | D | E | E | D | D | 95.5 |
| esculenta | | | | | | | | | | |
| Malus | Apple Tree | XP_008369314.1 | 83.737 | 242/289 | D | E | E | D | D | 94.81 |
| domestica | | | | | | | | | | |
| Cicer | ChickPea | XP_004491041.1 | 83.737 | 242/289 | D | E | E | D | D | 95.85 |
| arietinum | | | | | | | | | | |
| Cucumis | Muskmelon | XP_008456753.1 | 85.813 | 248/289 | D | E | E | D | D | 93.43 |
| melo | | | | | | | | | | |
| Pyrus × | Chinese | XP_009369241.1 | 83.045 | 240/289 | D | E | E | D | D | 94.81 |
| bretschneideri | white pear | | | | | | | | | |
| Corchorus | White Jute | OMO73552.1 | 84.88 | 247/291 | D | E | E | D | D | 92.44 |
| capsularis | | | | | | | | | | |
| Gossypium | Cotton Plant | XP_012489506.1 | 84.775 | 245/289 | D | E | E | D | D | 93.08 |
| raimondii | | | | | | | | | | |
| Prunus | Sweet/wild | XP_021824795.1 | 83.391 | 241/289 | D | E | E | D | D | 94.12 |
| avium | Cherry tree | | | | | | | | | |
| Lupinus | Lupin | OIW15090.1 | 88.176 | 261/296 | D | E | E | D | D | 93.58 |
| angustifolius | | | | | | | | | | |
| Gossypium | Extra long | PPR99271.1 | 79.677 | 247/310 | D | E | E | D | D | 89.35 |
| barbadense | staple cotton | | | | | | | | | |
| | (Sea Island | | | | | | | | | |
| | Cotton) | | | | | | | | | |
| Glycine soja | Wild Soybean | KHN38559.1 | 86.17 | 243/282 | D | E | E | D | D | 95.39 |
| Rosa | China | XP_024170812.1 | 83.737 | 242/289 | D | E | E | D | D | 93.43 |
| chinensis | rose/Chinese | | | | | | | | | |
| | rose | | | | | | | | | |
| Gossypium | Extra long | PPS02529.1 | 84.083 | 243/289 | D | E | E | D | D | 93.08 |
| barbadense | staple cotton | | | | | | | | | |
| | (Sea Island | | | | | | | | | |
| | Cotton) | | | | | | | | | |
| Parasponia | Parasponia | PON79077.1 | 87.889 | 254/289 | D | E | E | D | D | 96.89 |
| andersonii | andersonii | | | | | | | | | |
| Morus | Black | XP_024018217.1 | 87.889 | 254/289 | D | E | E | D | D | 96.19 |
| notabilis | Mulberry | | | | | | | | | |
| Jatropha | Jatropha | XP_012091205.1 | 84.083 | 243/289 | D | E | E | D | D | 94.12 |
| curcas | curcas | | | | | | | | | |
| Citrus | Clementine | XP_006435852.1 | 86.851 | 251/289 | D | E | E | D | D | 95.85 |
| clementina | | | | | | | | | | |
| Cephalotus | Australian | GAV62462.1 | 87.197 | 252/289 | D | E | E | D | D | 95.85 |
| follicularis | Pitcher Plant | | | | | | | | | |
| Durio | Durio | XP_022741218.1 | 87.543 | 253/289 | D | E | E | D | D | 95.16 |
| zibethinus | zibethinus | | | | | | | | | |
| Populus | Desert Poplar | XP_011005868.1 | 84.321 | 242/287 | D | E | E | D | D | 93.73 |
| euphratica | | | | | | | | | | |
| Populus | Black | XP_002312193.1 | 83.972 | 241/287 | D | E | E | D | D | 93.73 |
| trichocarpa | cottonwood | | | | | | | | | |
| Populus | Black | XP_006378643.2 | 83.624 | 240/287 | D | E | E | D | D | 94.43 |
| trichocarpa | cottonwood | | | | | | | | | |
| Gossypium | Cotton Tree | XP_017628059.1 | 83.391 | 241/289 | D | E | E | D | D | 92.73 |
| arboreum | | | | | | | | | | |
| Trema | Charcoal- | PON83245.1 | 87.543 | 253/289 | D | E | E | D | D | 96.54 |
| orientalis | tree/Indian | | | | | | | | | |
| | charcoal- | | | | | | | | | |
| | tree/pigeon | | | | | | | | | |
| | wood/Oriental | | | | | | | | | |
| | trema | | | | | | | | | |
| Cucumis | Cucumber | XP_004138403.1 | 84.429 | 244/289 | D | E | E | D | D | 92.73 |
| sativus | | | | | | | | | | |
| Gossypium | Mexican | XP_016708559.1 | 83.391 | 241/289 | D | E | E | D | D | 92.73 |
| hirsutum | Cotton Tree | | | | | | | | | |
| Cucurbita | Winter | XP_023515361.1 | 84.775 | 245/289 | D | E | E | D | D | 93.08 |
| pepo subsp. | Squash | | | | | | | | | |
| Pepo | | | | | | | | | | |
| Manihot | Yuca | XP_021626521.1 | 86.851 | 251/289 | D | E | E | D | D | 96.19 |
| esculenta | | | | | | | | | | |
| Durio | Durio | XP_022750516.1 | 87.591 | 240/274 | D | N/A | N/A | N/A | N/A | 94.16 |
| zibethinus | zibethinus | | | | | | | | | |
| Arachis | Wild | XP_015973743.1 | 82.007 | 237/289 | D | E | E | D | D | 94.46 |
| duranensis | Peanut | | | | | | | | | |
| Carica | Papaya | XP_021904215.1 | 87.889 | 254/289 | D | E | E | D | D | 94.46 |
| papaya | | | | | | | | | | |
| Arachis | Peanut | XP_016165661.1 | 81.661 | 236/289 | D | E | E | D | D | 94.12 |
| ipaensis | | | | | | | | | | |
| Cucurbita | Squash | XP_022991069.1 | 84.083 | 243/289 | D | E | E | D | D | 92.73 |
| maxima | | | | | | | | | | |
| Corchorus | Jute | OMO69109.1 | 83.162 | 242/291 | D | E | E | D | D | 91.41 |
| olitorius | Mallow | | | | | | | | | |
| Hevea | Rubberwood | XP_021668979.1 | 87.197 | 252/289 | D | E | E | D | D | 94.81 |
| brasiliensis | | | | | | | | | | |
| Populus | Desert Poplar | XP_011015133.1 | 82.578 | 237/287 | D | E | E | D | D | 94.08 |
| euphratica | | | | | | | | | | |
| Cucurbita | Pumpkin | XP_022964687.1 | 84.429 | 244/289 | D | E | E | D | D | 92.73 |
| moschata | | | | | | | | | | |
| Hevea | Rubberwood | XP_021688775.1 | 86.159 | 249/289 | D | E | E | D | D | 95.16 |
| brasiliensis | | | | | | | | | | |
| Erythranthe | Seep | XP_012840021.1 | 80.969 | 234/289 | D | E | E | D | D | 93.77 |
| guttata | monkeyflower/ | | | | | | | | | |
| | yellow | | | | | | | | | |
| | monkeyflower | | | | | | | | | |
| Sesamum | Sesame | XP_011084853.1 | 84.083 | 243/289 | D | E | E | D | D | 94.46 |
| indicum | | | | | | | | | | |
| Medicago | BarrelClover | XP_024639705.1 | 87.97 | 234/266 | D | N/A | N/A | N/A | N/A | 97.37 |
| truncatula | (small | | | | | | | | | |
| | Mediterranean | | | | | | | | | |
| | Legume) | | | | | | | | | |
| Ricinus | Castor bean | XP_002520820.1 | 85.813 | 248/289 | D | E | E | D | D | 95.5 |
| communis | or castor oil | | | | | | | | | |
| Ziziphus | Jujube red | XP_015877477.1 | 80.969 | 234/289 | D | E | D | D | D | 93.77 |
| jujuba | date | | | | | | | | | |
| Eucalyptus | Eucalyptus or | XP_010067574.1 | 81.661 | 236/289 | D | E | E | D | D | 93.43 |
| grandis | Rose Gum | | | | | | | | | |
| Cucurbita | Pumpkin | XP_022956354.1 | 80.969 | 234/289 | D | E | E | D | D | 93.77 |
| moschata | | | | | | | | | | |
| Cucurbita | Squash | XP_022991930.1 | 80.969 | 234/289 | D | E | E | D | D | 93.77 |
| maxima | | | | | | | | | | |
| Momordica | Bitter Melon | XP_022146873.1 | 80.969 | 234/289 | D | E | E | D | D | 93.43 |
| charantia | | | | | | | | | | |
| Morus | Black | EXB25858.1 | 81.613 | 253/310 | D | E | E | D | D | 89.68 |
| notabilis | Mulberry | | | | | | | | | |
| Malus | Apple Tree | XP_008374460.1 | 84.083 | 243/289 | D | E | E | D | D | 94.81 |
| domestica | | | | | | | | | | |
| Prunus | Peach | XP_007218769.1 | 83.391 | 241/289 | D | E | E | D | D | 93.77 |
| persica | | | | | | | | | | |
| Prunus | Japanese | XP_008233838.1 | 83.045 | 240/289 | D | E | E | D | D | 93.77 |
| mume | Apricot | | | | | | | | | |
| Sesamum | Sesame | XP_011076626.1 | 82.699 | 239/289 | D | E | E | D | D | 92.04 |
| indicum | | | | | | | | | | |
| Cucurbita | Squash | XP_022992586.1 | 85.467 | 247/289 | D | E | E | D | D | 94.46 |
| maxima | | | | | | | | | | |
| Momordica | Bitter Melon | XP_022134286.1 | 85.813 | 248/289 | D | E | E | D | D | 94.12 |
| charantia | | | | | | | | | | |
| Olea | Wild-olive | XP _022880461.1 | 81.661 | 236/289 | D | E | E | D | D | 92.73 |
| europaea | | | | | | | | | | |
| var. | | | | | | | | | | |
| sylvestris | | | | | | | | | | |
| Cucurbita | Pumpkin | XP_022939232.1 | 85.121 | 246/289 | D | E | E | D | D | 94.46 |
| moschata | | | | | | | | | | |
| Handroanthus | Pink Trumpet | PIN13349.1 | 82.007 | 237/289 | D | E | E | D | D | 91.7 |
| impetiginosus | Tree | | | | | | | | | |
| Nicotiana | Coyote | XP_019225807.1 | 79.585 | 230/289 | D | E | E | D | D | 92.39 |
| attenuata | Tobacco | | | | | | | | | |
| Punica | Pomegranate | PKI40618.1 | 78.547 | 227/289 | D | E | E | D | D | 91.35 |
| granatum | | | | | | | | | | |
| Nicotiana | Woodland | XP_009798526.1 | 79.585 | 230/289 | D | E | E | D | D | 92.73 |
| sylvestris | tobacco/ | | | | | | | | | |
| | Flowering | | | | | | | | | |
| | tobacco | | | | | | | | | |
| Nicotiana | Tobacco Plant | XP_009614295.1 | 79.585 | 230/289 | D | E | E | D | D | 92.73 |
| tomentosiformis | | | | | | | | | | |
| Erythranthe | Seep | XP_012858890.1 | 79.239 | 229/289 | D | E | D | D | D | 92.39 |
| guttata | monkeyflower/ | | | | | | | | | |
| | yellow | | | | | | | | | |
| | monkeyflower | | | | | | | | | |
| Solanum | Tomato | XP_004240900.1 | 79.585 | 230/289 | D | E | E | D | D | 92.04 |
| lycopersicum | | | | | | | | | | |

### Materials & Methods

### Recombinant Protein Production

Vectors encoding recombinant α-SNAP_{Rhg1}HC, α-SNAP_{Rhg1}LC, α-SNAP_{Rhg1}WT, α-SNAP_{Rhg1}WT₁₋₂₈₅ and the WT alleles of NSF Glyma.07G195900 (NSF_{Ch07}) and Glyma.13G180100 (NSF_{Chl3}) were generated in Bayless et al., 2016. The open reading frames (ORFs) encoding the soybean NSF_{RAN07} allele of Glyma.07G195900 or N. benthamiana NSF were cloned into the expression vector pRham N-His-SUMO Kan according to manufacturer instructions (Lucigen). Recombinant α-SNAP and NSF proteins were also produced and purified as in Bayless et al. 2016. All expression constructs were chemically transformed into the expression strain “E. cloni 10G” (Lucigen), grown to OD₆₀₀˜0.60-0.70, and induced with 0.2% L-Rhamnose (Sigma) for either 8 hr at 37° C. or overnight at 28° C. Soluble, native recombinant His-SUMO-α-SNAPs or His-SUMO-NSF proteins were purified with PerfectPro Ni-NTA resin (5 PRIME), and eluted with imidazole, though no subsequent gel filtration steps were performed. Following the elution of the His-SUMO-fusion proteins, overnight dialysis was performed at 4° C. in 20 mM Tris (pH 8.0), 150 mM NaCl, 10% (vol/vol) glycerol, and 1.5 mM Tris (2-carboxyethyl)-phosphine. The His-SUMO affinity/solubility tags were cleaved from α-SNAP or NSF using 1 or 2 units of SUMO Express protease (Lucigen) and separated by rebinding of the tag with Ni-NTA resin and collecting the recombinant protein from the flowthrough. Recombinant protein purity was assessed by Coomassie blue staining and quantified via a spectrophotometer.

### In Vitro NSF-α-SNAP Binding Assays

In vitro NSF binding assays were performed essentially as described in Barnard et. al. (1997) J Cell Biol 139(4): 875-883; and Bayless et al. (2016), Proc Natl Acad Sci USA 113(47): E7375-E7382; Briefly, 20 μg of each respective recombinant α-SNAP protein was added to the bottom of a 1.5-mL polypropylene tube and incubated at 25° C. for 20 min. Unbound α-SNAP proteins were then washed by adding α-SNAP wash buffer [25 mM Tris, pH 7.4, 50 mM KCl, 1 mM DTT, 0.4 mg/mL bovine serum albumin (BSA)]. After removal of wash buffer, 20 μg of recombinant NSF (1 μg/μL in NSF binding buffer), was then immediately added and incubated on ice for 10 min. The solution was then removed, and samples were immediately washed 2× with NBB to remove any unbound NSF. Samples were then boiled in 1×SDS loading buffer and separated on a 10% Bis-Tris SDS-PAGE, and silver-stained using the ProteoSilver Kit (Sigma-Aldrich), according to the manufacturer directions. The percentage of NSF bound by α-SNAP was then calculated using densitometric analysis with ImageJ.

### Antibody Production and Validation

Affinity-purified polyclonal rabbit antibodies raised against α-SNAP_{Rhg1}HC, α-SNAP_{Rhg1}LC and wild-type α-SNAPs were previously generated and validated using recombinant proteins in Bayless 2016. The epitopes for these custom antibodies are the final six or seven C-terminal α-SNAP residues: “EEDDLT” (SEQ ID NO: 127), “EQHEAIT” (SEQ ID NO: 128), or “EEYEVIT” (SEQ ID NO: 129) for wild-type, high-, or low-copy α-SNAPs, respectively. For NSF, a synthetic peptide, “ETEKNVRDLFADAEQDQRTRGDESD” (SEQ ID NO: 130), corresponding to residues 300 to 324 of Glyma.07G195900 was used. This NSF antibody was previously shown to be cross-reactive with the N. benthamiana-encoded NSF.

### Immunoblotting

Tissue preparation and immunoblots were performed essentially as in (Song et al., 2015a; Bayless et al., 2016). Soybean roots or N. benthamiana leaf tissues were flash-frozen in N₂(L), massed, and homogenized in a PowerLyzer 24 (MO BIO) for three cycles of 15 seconds, with flash-freezing in-between each cycle. Protein extraction buffer [50 mM Tris.HCl (pH 7.5), 150 mM NaCl, 5 mM EDTA, 0.2% Triton X-100, 10% (vol/vol) glycerol, 1/100 Sigma protease inhibitor cocktail] was then added at a 3:1 volume to mass ratio and samples were centrifuged and stored on ice. In noted experiments, Bradford assays were performed on each sample, and equal OD amounts of total protein were loaded in each sample lane for SDS/PAGE. Immunoblots for either Rhg1 α-SNAP were incubated overnight at 4° C. in 5% (wt/vol) nonfat dry milk TBS-T (50 mM Tris, 150 mM NaCl, 0.05% Tween 20) at 1:1,000. NSF immunoblots were performed similarly, except incubations were for 1 h at room temperature. Secondary horseradish peroxidase-conjugated goat anti-rabbit IgG was added at 1:10,000 and incubated for 1 h at room temperature on a platform shaker, followed by four washes with TBS-T. Chemiluminescence detection was performed with SuperSignal West Pico or Dura chemiluminescent substrate (Thermo Scientific) and developed using a ChemiDoc MP chemiluminescent imager (Bio-Rad).

### Transgenic Soybean Root Generation

Binary expression constructs were transformed into Agrobacterium rhizogenes strain, “Arqua1”. Transgenic soybean roots were produced as described in (Cook et al., 2012, Science 338, 1206-1209).

Transient Agrobacterium Expression in Nicotiana benthamiana. Agrobacterium tumefaciens strain GV3101 was used for transient protein expression of all constructs via syringe-infiltration at OD₆₀₀ 0.60 for NSF constructs or OD₆₀₀ 0.80 for α-SNAP constructs into young leaves of ˜4-wk-old N. benthamiana plants. GV3101 cultures were grown overnight at 28° C. in 25 μg/mL kanamycin and rifampicin and induced for ˜3.5 h in 10 mM Mes (pH 5.60), 10 mM MgCl2, and 100 μM acetosyringone prior to leaf infiltration. N. benthamiana plants were grown in a Percival set at 25° C. with a photoperiod of 16 h light at 100 μE·m-2·s-1 and 8 h dark. For α-SNAP complementation assays, GV3101 cultures were well-mixed with one volume of an empty vector control, or of the respective NSF construct immediately before co-infiltration. NSF_{RAN07} or the N. benthamiana NSF were PCR amplified from a root cDNA library of Rhg1_{LC} variety, “Forrest” or a N. benthamiana leaf cDNA library using KAPA HiFi polymerase, respectively. Expression cassettes for NSF_{N.benthamiana}, NSF_{Ch13}, NSF_{Ch07} and NSF_{RAN07} ORFs were directly assembled into a pBluescript vector containing the soybean ubiquitin (GmUbi) promoter and NOS terminator using Gibson assembly. The NSF expression cassettes were then digested with the restriction enzymes NotI-SalI and ligated with T4 DNA ligase into the previously described binary vector, pSM101-linker, which was cut with PspOMI-SalI restriction sites. The ORF encoding the α-SNAP_{Ch11} Intron-Retention (IR) allele was amplified with Kapa HiFi from a root cDNA library of Rhg1_{LC} variety “Forrest” while the ORF encoding WT α-SNAP_{Ch11} was previously generated in (Bayless et al., 2016, Proc. Natl. Acad. Sci. USA 113, E7375-E7382). Both α-SNAP_{Ch11} and α-SNAP_{Ch11} IR were Gibson assembled into a pBluescript vector containing a GmUbi-N-HA tag and NOS terminator, cut with PstI-XbaI and ligated into the binary vector, pSM101, cut with the same restriction pair. An 11.14 kb native genomic region encoding α-SNAP_{Rhg1}WT was amplified with Kapa HiFi from a previously described fosmid subclone (Fosmid 19) with AvrII-SbfI restriction ends, and then digested and ligated into the binary vector, pSM101, cut with XbaI-PstI. A 6.85 kb native locus encoding α-SNAP_{Chi I} was amplified from gDNA of Williams82 into two fragments (3.25 kb and 3.60 kb fragments) and Gibson assembled into pSM101 vector cut with BamHI-PstI.

### Protein Structure Modeling and Sequence Logo

NSFRAN07, α-SNAPCh11 and α-SNAPCh11IR structural homology models were generated using SWISS-MODEL and output PDB files viewed and labeled using PyMol. NSFRAN07 was modeled to NSFCHO (Chinese hamster ovary) (PDB 3j97.1) cryo-EM structure from Zhao et al (Brunger group). 20S supercomplex modeling also generated using PDB 3j97, with α-SNAPs and SNAREs of Rattus norvegicus origin (Zhao et al., 2015, Nature 518: 61-67). α-SNAPCh11 and α-SNAPCh11IR were modeled to sec17 (yeast α-SNAP) crystal structure 1QQE donated courtesy of Rice et al (Rice and Brunger, 1999, Mol Cell 4: 85-95).

The R4Q NSF amino acid consensus logo was generated using WebLogo. (Crooks G E, et al. (2004), Genome Res 14: 1188-1190).

### Whole-Genome Sequencing Data Analysis

Whole-genome sequencing data of 12 soybean varieties was obtained from previously published studies (Song et al., 2017, The Plant Genome 10); Cook et al., 2014 Plant Physiol 165, 630-647)). Illumina sequencing reads were aligned to the Williams 82 reference genome (Wm82.a2.v1; www.phytozome.org/) using BWA (version 0.7.12) (Li and Durbin, 2009, Bioinformatics, 25:1754-60). Reads were initially mapped using the default settings of the aln command with the subsequent pairings performed with the sampe command. Alignments were next processed using the program Picard (version 2.9.0) to add read group information (AddOrReplaceReadGroups), mark PCR duplicates (MarkDuplicates, and merge alignments from separate sequencing runs (MergeSamFiles). The processed .bam files were then converted to vcf format using a combination of samtools (version 0.1.19) and bcftools (version 0.1.19). Finally, consensus sequences were generated from these .vcf files using the FastaAlternateReferenceMaker tool within GATK (version 3.7.0; DePristo et al., 2011, Nat Genet 43: 491-498).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

| SEQ | Gene | |
|---|---|---|
| ID NO | Designator | Nucleotide Sequence |
| 1 | Glyma.18G022 | ATGTCTCCGGCCGCCGGAGTCAGCGTCCCCCTCCTGGGG |
| | 400 | GATTCCAAAGGAACGCCGCCGCCGGCTTCCGTCCCCGGC |
| | | GCGGTGTTCAACGTGGCCACCAGCATAGTCGGCGCCGGA |
| | | ATCATGTCGATTCCGGCGATCATGAAGGTTCTCGGCGTAG |
| | | TTCCCGCTTTCGCGATGATTCTCGTGGTGGCCGTGCTGGC |
| | | GGAACTGTCCGTGGACTTCCTGATGCGGTTCACGCACTCC |
| | | GGCGAAACGACGACGTACGCTGGCGTCATGAGGGAGGC |
| | | GTTCGGATCGGGTGGAGCATTgGCCGCGCAAGTTTGCGT |
| | | CATCATCACCAACGTTGGGGGTTTAATTCTCTACCTTATCA |
| | | TCATCGGAGATGTGCTATCTGGAAAGCAAAATGGAGGGGA |
| | | AGTGCATTTGGGCATTTTGCAACAGTGGTTTGGAATTCACT |
| | | GGTGGAATTCCCGGGAATTTGCTTTGCTTTTCACCTTGGT |
| | | CTTTGTTATGCTTCCATTGGTATTGTACAAACGTGTAGAGT |
| | | CCTTGAAGTACAGCTCTGCAGTGTCAACTCTTCTTGCAGT |
| | | GGCATTTGTTGGCATATGTTGTGGGTTGGCTATCACAGCT |
| | | CTGGTGCAAGGAAAAACACAAACTCCTAGATTGTTTCCTC |
| | | GGCTAGACTACCAAACCTCATTCTTTGATCTGTTCACTGCA |
| | | GTTCCTGTTGTTGTCACAGCCTTCACATTTCACTTTAATGT |
| | | GCACCCCATTGGGTTTGAGCTTGCCAAGGCATCCCAAATG |
| | | ACAACAGCAGTTCGATTAGCATTATTGCTTTGTGCTGTGAT |
| | | CTACCTTGCAATAGGCTTATTTGGGTACATGTTATTTGGGG |
| | | ATTCAACCCAGTCAGACATTCTCATCAATTTTGACCAGAAT |
| | | GCTGGTTCAGCAGTTGGTTCCTTGCTCAATAGTTTGGTCC |
| | | GTGTAAGCTATGCCCTCCACATCATGCTGGTGTTTCCTCT |
| | | CTTGAACTTCTCTTTGAGAACCAACATAGATGAAGTTCTCT |
| | | TCCCTAAGAAGCCTATGCTAGCCACAGACAACAAAAGATT |
| | | TATGATCCTCACTCTGGTGCTGCTTGTATTCTCCTACCTTG |
| | | CAGCTATAGCAATCCCAGATATTTGGTACTTCTTTCAGTTC |
| | | CTGGGATCCTCATCCGCAGTGTGCCTTGCCTTCATTTTCC |
| | | CCGGCTCTATTGTTTTAAGGGATGTTAAAGGTATATCAACG |
| | | AGAAGAGACAAAATTATTGCACTGATAATGATTATACTAGC |
| | | TGTGGTTACAAGTGTGCTTGCCATTTCCACCAACATATATA |
| | | ATGCTTTTAGTAGCAAGTCATAA |
| 2 | Glyma.18G022 | ATGGCCGATCAGTTATCGAAGGGAGAGGAATTCGAGAAAA |
| | 500 | AGGCTGAGAAGAAGCTCAGCGGTTGGGGCTTGTTTGGCT |
| | | CCAAGTATGAAGATGCCGCCGATCTCTTCGATAAAGCCGC |
| | | CAATTGCTTCAAGCTCGCCAAATCATGGGACAAGGCTGGA |
| | | GCGACATACCTGAAGTTGGCAAGTTGTCATTTGAAGTTGG |
| | | AAAGCAAGCATGAAGCTGCACAGGCCCATGTCGATGCTG |
| | | CACATTGCTACAAAAAGACTAATATAAACGAGTCTGTATCT |
| | | TGCTTAGACCGAGCTGTAAATCTTTTCTGTGACATTGGAAG |
| | | ACTCTCTATGGCTGCTAGATATTTAAAGGAAATTGCTGAAT |
| | | TGTACGAGGGTGAACAGAATATTGAGCAGGCTCTTGTTTA |
| | | CTATGAAAAATCAGCTGATTTTTTTCAAAATGAAGAAGTGA |
| | | CAACTTCTGCGAACCAATGCAAACAAAAAGTTGCCCAGTT |
| | | TGCTGCTCAGCTAGAACAATATCAGAAGTCGATTGACATTT |
| | | ATGAAGAGATAGCTCGCCAATCCCTCAACAATAATTTGCT |
| | | GAAGTATGGAGTTAAAGGACACCTTCTTAATGCTGGCATC |
| | | TGCCAACTCTGTAAAGAGGACGTTGTTGCTATAACCAATG |
| | | CATTAGAACGATATCAGGAACTGGATCCAACATTTTCAGG |
| | | AACACGTGAATATAGATTGTTGGCGGACATTGCTGCTGCA |
| | | ATTGATGAAGAAGATGTTGCAAAGTTTACTGATGTTGTCAA |
| | | GGAATTTGATAGTATGACCCCTCTGGATTCTTGGAAGACC |
| | | ACACTTCTCTTAAGGGTGAAGGAAAAGCTGAAAGCCAAAG |
| | | AACTTGAGGAGGATGATCTTACTTGA |
| 3 | Glyma.18G022 | ATGCGCATGCTCACCGGCGACTCCGCCGCCGACAACTCC |
| | 700 | TTCCGATTCGTTCCGCAGTCCATCGCCGCCTTCGGCTCCA |
| | | CCGTCATCGTCGAGGGCTGCGACTCCGCCCGCAACATTG |
| | | CCTGGGTCCACGCCTGGACCGTCACTGATGGGATGATCA |
| | | CTCAAATCAGAGAGTACTTCAACACCGCCCTCACCGTCAC |
| | | TCGCATCCACGATTCCGGCGAGATTGTTCCGGCCAGATCC |
| | | GGCGCCGGCCGTTTGCCCTGCGTCTGGGAGAGCAGCGT |
| | | CTCCGGTCGGGTCGGGAAATCCGTCCCCGGTTTGGTTCT |
| | | CGCAATATAA |
| 4 | Glyma.18G022 | ATGGTTTCGGTTGATGATGGGATTGTGAATCCCAATGATG |
| | 600 | AAATTGAGAAATCTAACGGGAGTAAAGTGAATGAGTTTGC |
| | | ATCTATGGATATTTCAGCAACTCAAAAATCATATCTGAACA |
| | | GTGAAGATCCTCAGAGAAGGCTTCAGGGAACCTTAATAAG |
| | | TTCTTCTGTTACTAATAGGATAAACTTTCTTAAATTTGGTTC |
| | | TGCATCTGCCAAATTCAAAAGGCTTGCTACTGAGAGAGAC |
| | | CAGGTTTCTATATCTGTGCCTTCTCCTCGTTCAAAGAGCCT |
| | | AAGATCACGTTTCAGTGGCATGTTTGCTCAGAAACTTGACT |
| | | GGGCTTCAGTCAAGAAAATGTGCATGGAATGGATTAGAAA |
| | | TCCAGTGAACATGGCCCTTTTTGTGTGGATCATTTGTGTC |
| | | GCGGTTTCGGGTGCTATTCTGTTCCTTGTCATGACAGGCA |
| | | TGTTGAATGGTGTGCTACCAAGAAAGTCTAAGAGAAATGC |
| | | ATGGTTTGAAGTAAACAACCAAATACTCAATGCAGTGTTTA |
| | | CACTCATGTGTTTGTACCAACACCCTAAGAGATTCTACCAC |
| | | CTTGTTCTTCTGACCAGATGAAGACCAAATGACATCTCTAG |
| | | CCTTAGGAAGGTATATTGCAAGAATGTCACTTACAAGCCC |
| | | CATGAGTGGACACATATGATGGTAGTTGTCATTCTCCTTCA |
| | | TGTTAACTGTTTTGCTCAATATGCACTTTGTGGTCTAAACT |
| | | TAGGGTATAAAAGGTCCGAGAGACCTGCCATTGGAGTTGG |
| | | AATATGCATATCTTTTGCAATTGCTGGTTTGTACACCATTC |
| | | TTAGCCCACTTGGGAAGGACTATGATTGTGAGATGGATGA |
| | | AGAAGCACAGGTTCAAATTACAGCTTCTCAAGGGAAAGAG |
| | | CAGCTGAGAGAGAAACCAACTGAGAAGAAATATTCATTTG |
| | | CATCCAAAGATCAACAAAGGGTTGTTGAAAATAGACCAAA |
| | | GTGGAGTGGAGGAATACTTGACATTTGGAACGATATTTCC |
| | | TTAGCATATCTCTCACTTTTCTGCACCTTTTGTGTGCTTGG |
| | | GTGGAATATGAAGAGGCTTGGCTTTGGAAACATGTATGTT |
| | | CACATTGCCATTTTTATGCTGTTCTGTATGGCTCCTTTCTG |
| | | GATTTTTCTTTTGGCTTCCGTTAACATAGATGATGACAATG |
| | | TTAGGCAGGCTCTAGCAGCTGTTGGAATCATTCTTTGTTTT |
| | | CTTGGTTTATTGTATGGTGGATTTTGGAGGATCCAAATGAG |
| | | AAAGAGGTTCAATTTACCAGCCTATGACTTCTGTTTTGGCA |
| | | AACCTTCAGCTTCTGATTGCACACTTTGGCTACCCTGTTGC |
| | | TGGTGCTCTCTCGCTCAAGAAGCGCGTACCAGGAATAACT |
| | | ATGATCTTGTAGAAGATAAATTCTCAAGGAAAGAAACTGAT |
| | | ACTAGTGATCAACCATCAATTTCACCTTTGGCTCGTGAAGA |
| | | TGTAGTGTCAACCAGATCTGGCACAAGTTCTCCTATGGGT |
| | | AGCACTAGCAACTCTTCCCCTTATATGATGAAAACATCTAG |
| | | TTCTCCAAATTCAAGCAATGTCTTAAAGGGATATTACAGTC |
| | | CAGATAAGATGCTATCAACTTTGAATGAAGACAATTGTGAA |
| | | AGAGGTCAAGATGGAACAATGAACCCCTTATATGCACAAA |
| | | AATAA |
| 5 | Glyma.18G022 | ATGGCCGATCAGTTATCGAAGGGAGAGGAATTCGAGAAAA |
| | 500. Fayette | AGGCTGAGAAGAAGCTCAGCGGTTGGGGCTTGTTTGGCT |
| | | CCAAGTATGAAGATGCCGCCGATCTCTTCGATAAAGCCGC |
| | | CAATTGCTTCAAGCTCGCCAAATCATGGGACAAGGCTGGA |
| | | GCGACATACCTGAAGTTGGCAAGTTGTCATTTGAAGTTGG |
| | | AAAGCAAGCATGAAGCTGCACAGGCCCATGTCGATGCTG |
| | | CACATTGCTACAAAAAGACTAATATAAACGAGTCTGTATCT |
| | | TGCTTAGACCGAGCTGTAAATCTTTTCTGTGACATTGGAAG |
| | | ACTCTCTATGGCTGCTAGATATTTAAAGGAAATTGCTGAAT |
| | | TGTACGAGGGTGAACAGAATATTGAGCAGGCTCTTGTTTA |
| | | CTATGAAAAATCAGCTGATTTTTTTCAAAATGAAGAAGTGA |
| | | CAACTTCTGCGAACCAATGCAAACAAAAAGTTGCCCAGTT |
| | | TGCTGCTCAGCTAGAACAATATCAGAAGTCGATTGACATTT |
| | | ATGAAGAGATAGCTCGCCAATCCCTCAACAATAATTTGCT |
| | | GAAGTATGGAGTTAAAGGACACCTTCTTAATGCTGGCATC |
| | | TGCAAACTCTGTAAAGAGGACGTTGTTGCTATAACCAATG |
| | | CATTAGAACGATATCAGGAACTGGATCCAACATTTTCAGG |
| | | AACACGTGAATATAGATTGTTGGCGGACATTGCTGCTGCA |
| | | ATTGATGAAGAAGATGTTGCAAAGTTTACTGATGTTGTCAA |
| | | GGAATTTGATAGTATGACCCCTCTGGATTCTTGGAAGACC |
| | | ACACTTCTCTTAAGGGTGAAGGAAAAGCTGAAAGCCAAAG |
| | | AACTTGAGCAGCATGAGGCTATTACTTGA |
| 6 | Glyma.18G022 | ATGGCCGATCAGTTATCGAAGGGAGAGGAATTCGAGAAAA |
| | 500 | AGGCTGAGAAGAAGCTCAGCGGTTGGGGCTTGTTTGGCT |
| | Peking | CCAAGTATGAAGATGCCGCCGATCTCTTCGATAAAGCCGC |
| | | CAATTGCTTCAAGCTCGCCAAATCATGGGACAAGGCTGGA |
| | | GCGACATACCTGAAGTTGGCAAGTTGTCATTTGAAGTTGG |
| | | AAAGCAAGCATGAAGCTGCACAGGCCCATGTCGATGCTG |
| | | CACATTGCTACAAAAAGACTAATATAAACGAGTCTGTATCT |
| | | TGCTTAGACCGAGCTGTAAATCTTTTCTGTGACATTGGAAG |
| | | ACTCTCTATGGCTGCTAGATATTTAAAGGAAATTGCTGAAT |
| | | TGTACGAGGGTGAACAGAATATTGAGCAGGCTCTTGTTTA |
| | | CTATGAAAAATCAGCTGATTTTTTTCAAAATGAAGAAGTGA |
| | | CAACTTCTGCGAACCAATGCAAACAAAAAGTTGCCCAGTT |
| | | TGCTGCTCAGCTAGAACAATATCAGAAGTCGATTGACATTT |
| | | ATGAAGAGATAGCTCGCCAATCCCTCAACAATAATTTGCT |
| | | GAAGTATGGAGTTAAAGGACACCTTCTTAATGCTGGCATC |
| | | TGCCAACTCTGTAAAGAGGAGGTTGTTGCTATAACCAATG |
| | | CATTAGAACGATATCAGGAACTGGATCCAACATTTTCAGG |
| | | AACACGTGAATATAGATTGTTGGCGGACATTGCTGCTGCA |
| | | ATTGATGAAGAAGATGTTGCAAAGTTTACTGATGTTGTCAA |
| | | GGAATTTGATAGTATGACCCCTCTGGATTCTTGGAAGACC |
| | | ACACTTCTCTTAAGGGTGAAGGAAAAGCTGAAAGCCAAAG |
| | | AACTTGAGGAGTATGAGGTTATTACTTGA |
| 7 | Glyma.18G022 | ATGGCCGATCAGTTATCGAAGGGAGAGGAATTCGAGAAAA |
| | 500 | AGGCTGAGAAGAAGCTCAGCGGTTGGGGCTTGTTTGGCT |
| | Peking Iso | CCAAGTATGAAGATGCCGCCGATCTCTTCGATAAAGCCGC |
| | | CAATTGCTTCAAGCTCGCCAAATCATGGGACAAGGCTGGA |
| | | GCGACATACCTGAAGTTGGCAAGTTGTCATTTGAAGTTGG |
| | | AAAGCAAGCATGAAGCTGCACAGGCCCATGTCGATGCTG |
| | | CACATTGCTACAAAAAGACTAATATAAACGAGTCTGTATCT |
| | | TGCTTAGACCGAGCTGTAAATCTTTTCTGTGACATTGGAAG |
| | | ACTCTCTATGGCTGCTAGATATTTAAAGGAAATTGCTGAAT |
| | | TGTACGAGGGTGAACAGAATATTGAGCAGGCTCTTGTTTA |
| | | CTATGAAAAATCAGCTGATTTTTTTCAAAATGAAGAAGTGA |
| | | CAACTTCTGCGAACCAATGCAAACAAAAAGTTGCCCAGTT |
| | | TGCTGCTCAGCTAGAACAATATCAGAAGTCGATTGACATTT |
| | | ATGAAGAGATAGCTCGCCAATCCCTCAACAATAATTTGCT |
| | | GAAGTATGGAGTTAAAGGACACCTTCTTAATGCTGGCATC |
| | | TGCCAACTCTGTAAAGAGGAGGAACTGGATCCAACATTTT |
| | | CAGGAACACGTGAATATAGATTGTTGGCGGACATTGCTGC |
| | | TGCAATTGATGAAGAAGATGTTGCAAAGTTTACTGATGTTG |
| | | TCAAGGAATTTGATAGTATGACCCCTCTGGATTCTTGGAA |
| | | GACCACACTTCTCTTAAGGGTGAAGGAAAAGCTGAAAGCC |
| | | AAAGAACTTGAGGAGTATGAGGTTATTACTTGA |
| 8 | Glyma.07G195 | ATGGCGAGTCGGTTCGGGTTATCGTCTTCGTCTTCCTCTGCGTC |
| | 900 WT | CAGCATGAGAGTTACCAACACGCCCGCGAGCGACCTCGCCCTC |
| | | ACCAACCTCGCCTTCTGTTCCCCCTCCGATCTCCGCAATTTCGC |
| | | CGTCCCTGGCCACAATAACCTCTACCTCGCCGCCGTCGCCGATT |
| | | CCTTCGTCTTATCTCTCTCTGCTCATGACACCATAGGCAGCGGT |
| | | CAGATTGCGTTGAATGCCGTTCAACGCCGGTGTGCCAAAGTTTC |
| | | TTCCGGTGATTCCGTACAAGTGAGCCGATTTGTGCCGCCTGAAG |
| | | ATTTCAACCTCGCACTGCTAACTCTTGAATTGGAATTTGTTAAAA |
| | | AGGGGAGTAAGAGTGAGCAGATTGATGCTGTTCTACTGGCTAAG |
| | | CAACTTCGTAAGAGATTTATGAACCAGGTTATGACTGTGGGGCA |
| | | GAAAGTATTATTTGAGTATCACGGAAATAATTATAGCTTTACTGT |
| | | CAGTAATGCTGCTGTTGAGGGCCAAGAAAAGTCTAATTCTCTTG |
| | | AAAGAGGGATGATTTCAGATGACACATACATTGTTTTTGAAACAT |
| | | CACGTGATAGTGGAATTAAGATTGTCAATCAACGAGAGGGTGCC |
| | | ACTAGCAACATTTTCAAGCAGAAAGAATTTAACCTTCAGTCACTG |
| | | GGTATTGGTGGCCTGAGTGCAGAATTTGCAGATATATTTCGAAG |
| | | AGCTTTTGCCTCTCGTGTTTTCCCACCCCATGTGACATCTAAATT |
| | | AGGAATCAAGCATGTGAAGGGCATGCTTCTTTATGGGCCTCCTG |
| | | GAACTGGAAAGACACTTATGGCACGCCAAATTGGAAAAATTTTG |
| | | AATGGGAAGGAACCCAAGATTGTAAATGGCCCTGAAGTTTTGAG |
| | | CAAATTTGTTGGTGAAACTGAAAAGAATGTGAGAGACCTTTTTGC |
| | | TGATGCTGAACAGGATCAGAGGACCCGAGGGGATGAAAGTGAT |
| | | TTGCATGTTATAATCTTTGATGAAATTGATGCTATTTGCAAGTCAA |
| | | GAGGTTCAACTCGAGATGGTACTGGAGTTCATGATAGTATTGTA |
| | | AATCAGCTTCTTACTAAGATAGATGGTGTGGAGTCACTAAATAAT |
| | | GTTTTACTTATTGGAATGACTAACAGAAAGGACATGCTTGATGAA |
| | | GCTCTCTTAAGACCAGGGAGGTTGGAAGTCCAGGTTGAGATAAG |
| | | CCTTCCTGATGAAAATGGTCGATTGCAAATTCTTCAAATCCATAC |
| | | TAACAAAATGAAAGAGAATTCTTTTCTAGCTGCTGATGTGAACCT |
| | | TCAAGAGCTTGCTGCTCGAACGAAAAACTACAGTGGTGCAGAAC |
| | | TTGAAGGTGTTGTGAAAAGTGCTGTCTCATATGCTTTAAATAGAC |
| | | AATTGAGTCTAGAGGATCTCACTAAGCCAGTGGAGGAAGAGAAC |
| | | ATTAAGGTTACAATGGATGACTTTTTGAATGCACTCCACGAAGTT |
| | | ACTTCCGCATTTGGAGCTTCAACTGATGATCTTGAAAGATGCAG |
| | | ACTCCATGGCATGGTTGAGTGTGGTGATCGACATAAGCACATTT |
| | | ATCAAAGAGCAATGCTACTTGTGGAGCAAGTTAAAGTGAGTAAA |
| | | GGAAGCCCACTTGTCACTTGTCTCCTGGAAGGTTCCCGTGGCA |
| | | GTGGTAAAACTGCACTTTCAGCTACTGTTGGTATCGACAGCGAC |
| | | TTCCCATACGTCAAGATAGTTTCAGCTGAATCAATGATTGGTCTA |
| | | CATGAGAGCACCAAATGTGCACAGATTATTAAGGTTTTTGAGGAT |
| | | GCATACAAGTCACCATTGAGTGTCATCATTCTTGATGACATTGAG |
| | | AGATTATTGGAGTATGTGCCCATTGGTCCTCGATTTTCAAACTTG |
| | | ATTTCTCAGACACTGCTGGTTCTGCTCAAACGGCTTCCTCCAAA |
| | | GGGGAAAAAACTAATGGTTATTGGCACAACAAGTGAACTAGATT |
| | | TCTTGGAATCAATTGGATTTTGTGATACCTTCTCTGTTACTTACCA |
| | | TATTCCTACCTTGAACACAACGGATGCAAAGAAGGTCCTAGAAC |
| | | AGTTGAATGTGTTTACTGATGAAGATATTGATTCTGCTGCAGAGG |
| | | CGTTGAATGATATGCCTATCAGGAAACTATACATGTTGATCGAGA |
| | | TGGCAGCGCAAGGGGAGCATGGTGGATCTGCAGAAGCCATCTT |
| | | TTCTGGCAAAGAGAAGATTAGTATCGCTCATTTCTATGATTGCCT |
| | | CCAGGATGTTGTTAGGTTATAA |
| 9 | Glyma.07G195 | ATGGCGAGTCAGTTCGGGTTATCGTCTTCGTCTTCCTCTGCGTC |
| | 900 RAN07 | CAGCATGAGAGTTACCTACACGCCCGCGAACGACCTCGCCCTC |
| | | ACCAACCTCGCCTTCTGTTCCCCCTCCGATCTCCGCAATTTCGC |
| | | CGTCCCTGGCCACAATAACCTCTACCTCGCCGCCGTCGCCGATT |
| | | CCTTCGTCTTATCTCTCTCTGCTCATGACACCATAGGCAGCGGT |
| | | CAGATTGCGTTGAATGCCGTTCAACGCCGGTGTGCCAAAGTTTC |
| | | TTCCGGTGATTCCGTACAAGTGAGCCGATTTGTGCCGCCTGAAG |
| | | ATTTCAACCTCGCACTGCTAACTCTTGAATTGGAATTTTTTGTTAA |
| | | AAAGGGGAGTAAGAGTGAGCAGATTGATGCTGTTCTACTGGCTA |
| | | AGCAACTTCGTAAGAGATTTATGAACCAGGTTATGACTGTGGGG |
| | | CAGAAAGTATTATTTGAGTATCACGGAAATAATTATAGCTTTACT |
| | | GTCAGTAATGCTGCTGTTGAGGGCCAAGAAAAGTCTAATTCTCT |
| | | TGAAAGAGGGATTATTTCAGATGACACATACATTGTTTTTGAAAC |
| | | ATCACGTGATAGTGGAATTAAGATTGTCAATCAACGAGAGGGTG |
| | | CCACTAGCAACATTTTCAAGCAGAAAGAATTTAACCTTCAGTCAC |
| | | TGGGTATTGGTGGCCTGAGTGCAGAATTTGCAGATATATTTCGA |
| | | AGAGCTTTTGCCTCTCGTGTTTTCCCACCCCATGTGACATCTAAA |
| | | TTAGGGATCAAGCATGTGAAGGGCATGCTTCTTTATGGGCCTCC |
| | | TGGAACTGGAAAGACACTTATGGCACGCCAAATTGGAAAAATTT |
| | | TGAATGGGAAGGAACCCAAGATTGTAAATGGCCCTGAAGTTTTG |
| | | AGCAAATTTGTTGGTGAAACTGAAAAGAATGTGAGAGACCTTTTT |
| | | GCTGATGCTGAACAGGATCAGAGGACCCGAGGGGATGAAAGTG |
| | | ATTTGCATGTTATAATCTTTGATGAAATTGATGCTATTTGCAAGTC |
| | | AAGAGGTTCAACTCGAGATGGTACTGGAGTTCATGATAGTATTG |
| | | TAAATCAGCTTCTTACTAAGATAGATGGTGTGGAGTCACTAAATA |
| | | ATGTTTTACTTATTGGAATGACTAACAGAAAGGACATGCTTGATG |
| | | AAGCTCTCTTAAGACCAGGGAGGTTGGAAGTCCAGGTTGAGATA |
| | | AGCCTTCCTGATGAAAATGGTCGATTGCAAATTCTTCAAATTCAT |
| | | ACTAACAAAATGAAAGAGAATTCTTTTCTAGCTGCTGATGTGAAC |
| | | CTTCAAGAGCTTGCTGCTCGAACGAAAAACTACAGTGGTGCAGA |
| | | ACTTGAAGGTGTTGTGAAAAGTGCTGTCTCATATGCTTTAAATAG |
| | | ACAATTGAGTCTAGAGGATCTCACTAAGCCAGTGGAGGAAGAGA |
| | | ACATTAAGGTTACAATGGATGACTTTTTGAATGCACTCCACGAAG |
| | | TTACTTCCGCATTTGGAGCTTCAACTGATGATCTTGAAAGATGCA |
| | | GACTCCATGGCATGGTTGAGTGTGGTGATCGACATAAGCACATT |
| | | TATCAAAGAGCAATGCTACTTGTGGAGCAAGTTAAAGTGAGTAA |
| | | AGGAAGCCCACTTGTCACTTGTCTCCTGGAAGGTTCCCGTGGCA |
| | | GTGGTAAAACTGCACTTTCAGCTACTGTTGGTATCGACAGCGAC |
| | | TTCCCATACGTCAAGATAGTTTCAGCTGAATCAATGATTGGTCTA |
| | | CATGAGAGCACCAAATGTGCACAGATTATTAAGGTTTTTGAGGAT |
| | | GCATACAAGTCACCATTGAGTGTCATCATTCTTGATGACATTGAG |
| | | AGATTATTGGAGTATGTGCCCATTGGTCCTCGATTTTCAAACTTG |
| | | ATTTCTCAGACACTGCTGGTTCTGCTCAAACGCTTCCTCCAAAG |
| | | GGGAAAAAACTCATGGTTATTGGCACAACAAGTGAACTAGATTT |
| | | CTTGGAATCAATTGGATTTTGTGATACCTTCTCTGTTACTTACCAT |
| | | ATTCCTACCTTGAACACAACGGATGCAAAGAAGGTCCTAGAACA |
| | | GTTGAATGTGTTTACTGATGAAGATATTGATTCTGCTGCAGAGGC |
| | | GTTGAATGATATGCCTATCAGGAAACTATACATGTTGATCGAGAT |
| | | GGCAGCGCAAGGGGAGCATGGTGGATCTGCAGAAGCCATCTTT |
| | | TCTGGCAAAGAGAAGATTAGTATCGCTCACTTCTATGATTGCCTC |
| | | CAGGATGTTGTTAGGTTATGA |
| 10 | Glyma.18G022400 | MSPAAGVSVPLLGDSKGTPPPASVPGAVFNVATSIVGAG |
| | | IMSIPAIMKVLGVVPAFAMILVVAVLAELSVDFLMRFTHSG |
| | | ETTTYAGVMREAFGSGGALAAQVCVIITNVGGLILYLIIIGD |
| | | VLSGKQNGGEVHLGILQQWFGIHWWNSREFALLFTLVFV |
| | | MLPLVLYKRVESLKYSSAVSTLLAVAFVGICCGLAITALVQ |
| | | GKTQTPRLFPRLDYQTSFFDLFTAVPVVVTAFTFHFNVHP |
| | | IGFELAKASQMTTAVRLALLLCAVIYLAIGLFGYMLFGDST |
| | | QSDILINFDQNAGSAVGSLLNSLVRVSYALHIMLVFPLLNF |
| | | SLRTNIDEVLFPKKPMLATDNKRFMILTLVLLVFSYLAAIAI |
| | | PDIWYFFQFLGSSSAVCLAFIFPGSIVLRDVKGISTRRDKII |
| | | ALIMIILAVVTSVLAISTNIYNAFSSKS |
| 11 | Glyma.18G022500 | MADQLSKGEEFEKKAEKKLSGWGLFGSKYEDAADLFDK |
| | | AANCFKLAKSWDKAGATYLKLASCHLKLESKHEAAQAHV |
| | | DAAHCYKKTNINESVSCLDRAVNLFCDIGRLSMAARYLKE |
| | | IAELYEGEQNIEQALVYYEKSADFFQNEEVTTSANQCKQK |
| | | VAQFAAQLEQYQKSIDIYEEIARQSLNNNLLKYGVKGHLL |
| | | NAGICQLCKEDVVAITNALERYQELDPTFSGTREYRLLADI |
| | | AAAIDEEDVAKFTDVVKEFDSMTPLDSWKTTLLLRVKEKL |
| | | KAKELEEDDLT |
| 12 | Glyma.18G022700 | MRMLTGDSAADNSFRFVPQSIAAFGSTVIVEGCDSARNIA |
| | | WVHAWTVTDGMITQIREYFNTALTVTRIHDSGEIVPARSG |
| 13 | Glyma.18G022600 | MVSVDDGIVNPNDEIEKSNGSKVNEFASMDISATQKSYL |
| | | NSEDPQRRLQGTLISSSVTNRINFLKFGSASAKFKRLATE |
| | | RDQVSISVPSPRSKSLRSRFSGMFAQKLDWASVKKMCM |
| | | EWIRNPVNMALFVWIICVAVSGAILFLVMTGMLNGVLPRK |
| | | SKRNAWFEVNNQILNAVFTLIPNDISSLRKVYCKNVTYKP |
| | | HEWTHMMVVVILLHVNCFAQYALCGLNLGYKRSERPAIG |
| | | VGICISFAIAGLYTILSPLGKDYDCEMDEEAQVQITASQGK |
| | | EQLREKPTEKKYSFASKDQQRVVENRPKVVSGGILDIWN |
| | | DISLAYLSLFCTFCVLGWNMKRLGFGNMYVHIAIFMLFCM |
| | | APFWIFLLASVNIDDDNVRQALAAVGIILCFLGLLYGGFWR |
| | | IQMRKRFNLPAYDFCFGKPSASDCTLWLPCCWCSLAQE |
| | | ARTRNNYDLVEDKFSRKETDTSDQPSISPLAREDVVSTR |
| | | SGTSSPMGSTSNSSPYMMKTSSSPNSSNVLKGYYSPDK |
| | | MLSTLNEDNCERGQDGTMNPLYAQK |
| 14 | Glyma.18G022500. | MADQLSKGEEFEKKAEKKLSGWGLFGSKYEDAADLFDK |
| | Fayette | AANCFKLAKSWDKAGATYLKLASCHLKLESKHEAAQAHV |
| | | DAAHCYKKTNINESVSCLDRAVNLFCDIGRLSMAARYLKE |
| | | IAELYEGEQNIEQALVYYEKSADFFQNEEVTTSANQCKQK |
| | | VAQFAAQLEQYQKSIDIYEEIARQSLNNNLLKYGVKGHLL |
| | | NAGICKLCKEDVVAITNALERYQELDPTFSGTREYRLLADI |
| | | AAAIDEEDVAKFTDVVKEFDSMTPLDSWKTTLLLRVKEKL |
| | | KAKELEQHEAIT |
| 15 | Glyma.18G022500 | MADQLSKGEEFEKKAEKKLSGWGLFGSKYEDAADLFDK |
| | Peking | AANCFKLAKSWDKAGATYLKLASCHLKLESKHEAAQAHV |
| | | DAAHCYKKTNINESVSCLDRAVNLFCDIGRLSMAARYLKE |
| | | IAELYEGEQNIEQALVYYEKSADFFQNEEVTTSANQCKQK |
| | | VAQFAAQLEQYQKSIDIYEEIARQSLNNNLLKYGVKGHLL |
| | | NAGICQLCKEEVVAITNALERYQELDPTFSGTREYRLLADI |
| | | AAAIDEEDVAKFTDVVKEFDSMTPLDSWKTTLLLRVKEKL |
| | | KAKELEEYEVIT |
| 16 | Glyma.18G022500 | MADQLSKGEEFEKKAEKKLSGWGLFGSKYEDAADLFDK |
| | Peking Iso | AANCFKLAKSWDKAGATYLKLASCHLKLESKHEAAQAHV |
| | | DAAHCYKKTNINESVSCLDRAVNLFCDIGRLSMAARYLKE |
| | | IAELYEGEQNIEQALVYYEKSADFFQNEEVTTSANQCKQK |
| | | VAQFAAQLEQYQKSIDIYEEIARQSLNNNLLKYGVKGHLL |
| | | NAGICQLCKEEELDPTFSGTREYRLLADIAAAIDEEDVAKF |
| | | TDVVKEFDSMTPLDSWKTTLLLRVKEKLKAKELEEYEVIT |
| 17 | Glyma.07G195900 | MASRFGLSSSSSSASSMRVTNTPASDLALTNLAFCSPSD |
| | WT | LRNFAVPGHNNLYLAAVADSFVLSLSAHDTIGSGQIALNA |
| | | VQRRCAKVSSGDSVQVSRFVPPEDFNLALLTLELEFVKK |
| | | GSKSEQIDAVLLAKQLRKRFMNQVMTVGQKVLFEYHGN |
| | | NYSFTVSNAAVEGQEKSNSLERGMISDDTYIVFETSRDS |
| | | GIKIVNQREGATSNIFKQKEFNLQSLGIGGLSAEFADIFRR |
| | | AFASRVFPPHVTSKLGIKHVKGMLLYGPPGTGKTLMARQI |
| | | GKILNGKEPKIVNGPEVLSKFVGETEKNVRDLFADAEQD |
| | | QRTRGDESDLHVIIFDEIDAICKSRGSTRDGTGVHDSIVN |
| | | QLLTKIDGVESLNNVLLIGMTNRKDMLDEALLRPGRLEVQ |
| | | VEISLPDENGRLQILQIHTNKMKENSFLAADVNLQELAAR |
| | | TKNYSGAELEGVVKSAVSYALNRQLSLEDLTKPVEEENIK |
| | | VTMDDFLNALHEVTSAFGASTDDLERCRLHGMVECGDR |
| | | HKHIYQRAMLLVEQVKVSKGSPLVTCLLEGSRGSGKTAL |
| | | SATVGIDSDFPYVKIVSAESMIGLHESTKCAQIIKVFEDAY |
| | | KSPLSVIILDDIERLLEYVPIGPRFSNLISQTLLVLLKRLPPK |
| | | GKKLMVIGTTSELDFLESIGFCDTFSVTYHIPTLNTTDAKK |
| | | VLEQLNVFTDEDIDSAAEALNDMPIRKLYMLIEMAAQGEH |
| | | GGSAEAIFSGKEKISIAHFYDCLQDVVRL |
| 18 | Glyma.07G195900 | MASQFGLSSSSSSASSMRVTYTPANDLALTNLAFCSPSD |
| | RAN07 | LRNFAVPGHNNLYLAAVADSFVLSLSAHDTIGSGQIALNA |
| | | VQRRCAKVSSGDSVQVSRFVPPEDFNLALLTLELEFFVK |
| | | KGSKSEQIDAVLLAKQLRKRFMNQVMTVGQKVLFEYHG |
| | | NNYSFTVSNAAVEGQEKSNSLERGIISDDTYIVFETSRDS |
| | | GIKIVNQREGATSNIFKQKEFNLQSLGIGGLSAEFADIFRR |
| | | AFASRVFPPHVTSKLGIKHVKGMLLYGPPGTGKTLMARQI |
| | | GKILNGKEPKIVNGPEVLSKFVGETEKNVRDLFADAEQD |
| | | QRTRGDESDLHVIIFDEIDAICKSRGSTRDGTGVHDSIVN |
| | | QLLTKIDGVESLNNVLLIGMTNRKDMLDEALLRPGRLEVQ |
| | | VEISLPDENGRLQILQIHTNKMKENSFLAADVNLQELAAR |
| | | TKNYSGAELEGVVKSAVSYALNRQLSLEDLTKPVEEENIK |
| | | VTMDDFLNALHEVTSAFGASTDDLERCRLHGMVECGDR |
| | | HKHIYQRAMLLVEQVKVSKGSPLVTCLLEGSRGSGKTAL |
| | | SATVGIDSDFPYVKIVSAESMIGLHESTKCAQIIKVFEDAY |
| | | KSPLSVIILDDIERLLEYVPIGPRFSNLISQTLLVLLKRLPPK |
| | | GKKLMVIGTTSELDFLESIGFCDTFSVTYHIPTLNTTDAKK |
| | | VLEQLNVFTDEDIDSAAEALNDMPIRKLYMLIEMAAQGEH |
| | | GGSAEAIFSGKEKISIAHFYDCLQDVVRL |
| 19 | NSF from Chinese | MAGRSMQAARCPTDELSLSNCAVVSEKDYQSGQHVIVR |
| | Hamster Ovary | TSPNHKYIFTLRTHPSVVPGSVAFSLPQRKWAGLSIGQEI |
| | Cells (Cricetulus | EVALYSFDKAKQCIGTMTIEIDFLQKKNIDSNPYDTDKMAA |
| | griseus) | EFIQQFNNQAFSVGQQLVFSFNDKLFGLLVKDIEAMDPSI |
| | | LKGEPASGKRQKIEVGLVVGNSQVAFEKAENSSLNLIGKA |
| | | KTKENRQSIINPDWNFEKMGIGGLDKEFSDIFRRAFASRV |
| | | FPPEIVEQMGCKHVKGILLYGPPGCGKTLLARQIGKMLNA |
| | | REPKVVNGPEILNKYVGESEANIRKLFADAEEEQRRLGA |
| | | NSGLHIIIFDEIDAICKQRGSMAGSTGVHDTVVNQLLSKID |
| | | GVEQLNNILVIGMTNRPDLIDEALLRPGRLEVKMEIGLPDE |
| | | KGRLQILHIHTARMRGHQLLSADVDIKELAVETKNFSGAE |
| | | LEGLVRAAQSTAMNRHIKASTKVEVDMEKAESLQVTRGD |
| | | FLASLENDIKPAFGTNQEDYASYIMNGIIKWGDPVTRVLD |
| | | DGELLVQQTKNSDRTPLVSVLLEGPPHSGKTALAAKIAEE |
| | | SNFPFIKICSPDKMIGFSETAKCQAMKKIFDDAYKSQLSC |
| | | VVVDDIERLLDYVPIGPRFSNLVLQALLVLLKKAPPQGRKL |
| | | LIIGTTSRKDVLQEMEMLNAFSTTIHVPNIATGEQLLEALEL |
| | | LGNFKDKERTTIAQQVKGKKVWIGIKKLLMLIEMSLQMDP |
| | | EYRVRKFLALLREEGASPLDFD |

What is claimed is:

1. A method of producing plant cells with enhanced nematode resistance, comprising:
a) increasing expression of, altering an expression pattern of, altering a polynucleotide sequence of, altering abundance or localization of a polypeptide product of, or increasing copy number of,
(i) one or more polynucleotides encoding alpha-soluble N-ethylmaleimide-sensitive factor Attachment Protein (α-SNAP), or resistance-promoting variants thereof, or
(ii) one or more polynucleotides encoding soluble N-ethylmaleimide-sensitive factor (NSF) proteins, or homologs or variants thereof,
wherein the plant cells exhibit increased resistance to nematodes.

2. The method of claim 1, wherein,
a polynucleotide encoding one or more α-SNAP proteins has at least 75% identity to a polynucleotide identified by SEQ ID NOs: 2, 5 or 6, or
an encoded polypeptide has at least 75% identity to a polypeptide identified by SEQ ID NOs: 11, 14 or 15, or homologs or variants thereof, and
a polynucleotide encoding one or more NSF proteins has at least 75% identity to a polynucleotide identified by SEQ ID NOS: 8 or 9, or
an encoded polypeptide has at least 75% identity to a polypeptide identified by SEQ ID NOs 17 or 18, or homologs or variants thereof.

3. The method of claim 1, wherein the one or more polynucleotides encodes a modified α-SNAP polypeptide, wherein:
the modified α-SNAP polypeptide comprises one or a plurality of amino acid modifications at positions corresponding to positions 203, 208, 285, 286, 287, and 288 with numbering relative to the α-SNAP polypeptide set forth in SEQ ID NO: 11 or to positions 203, 208, 285, 286, 287, 288, or 289 with numbering relative to the α-SNAP set forth in SEQ ID NOS: 14 or 15;
the modified α-SNAP polypeptide comprises the amino acid modification or amino acid modifications compared to the α-SNAP set forth in SEQ ID NOS 11, 14, or 15; whereby the modified α-SNAP polypeptide comprises a sequence of amino acids that has less than 100% identity or has 100% identity to the modified and more than 75% identity to the α-SNAP polypeptide as set forth in SEQ ID NO 11; and the modified α-SNAP polypeptide comprises a sequence of amino acids that has greater than 75% sequence identity to the α-SNAP set forth in SEQ ID NOS: 11; and
the modified α-SNAP confers enhanced nematode resistance in the plant cell that is greater than the nematode resistance in the plant cell without the α-SNAP amino acid modification or amino acid modifications.

4. The method of claim 3, wherein the encoded modified α-SNAP comprises amino acid modifications at positions corresponding to positions 208, 285, 286, 287, and 288 by α-SNAP numbering relative to position in the α-SNAP polypeptide set forth in SEQ ID NO: 11.

5. The method of claim 3, wherein the modified polynucleotides encode a modified α-SNAP polypeptide, wherein the modified α-SNAP polypeptide comprises:
a replacement at position D286 that is D286F, or D286W, or D286Y; and
a replacement at position D287 that is D287E or remains D287; and
an insertion after position 287 that is (ins)288A, (ins)288G, (ins)2881, (ins)288L, (ins)288M, or (ins)288V; and
a replacement at position L288 that is L288A, L288G, L2881, L2881, L288M, or L288V, or
a functional equivalent amino acid to the WT amino acid expressed at position 285, 286, 287, or 288, each by α-SNAP numbering relative to the positions set for in SEQ ID NO: 11.

6. The method of claim 5, wherein the encoded modified NSF polypeptide comprises same family amino acid modifications selected from among modifications corresponding to:
D286F/D287E/(del)288A/L289A;
D286F/D287E/(del)288A/L289G;
D286F/D287E/(del)288A/L2891;
D286F/D287E/(del)288A/L289L;
D286F/D287E/(del)288A/L289M;
D286F/D287E/(del)288A/L289V;
D286F/D287E/(del)288G/L289A;
D286F/D287E/(del)288G/L289G;
D286F/D287E/(del)288G/L2891;
D286F/D287E/(del)288G/L289L;
D286F/D287E/(del)288G/L289M;
D286F/D287E/(del)288G/L289V
D286F/D287E/(del)2881/L289A;
D286F/D287E/(del)2881/L289G;
D286F/D287E/(del)2881/L2891;
D286F/D287E/(del)2881/L289L;
D286F/D287E/(del)2881/L289M;
D286F/D287E/(del)2881/L289V;
D286F/D287E/(del)288L/L289A;
D286F/D287E/(del)288L/L289G;
D286F/D287E/(del)288L/L2891;
D286F/D287E/(del)288L/L289L;
D286F/D287E/(del)288L/L289M;
D286F/D287E/(del)288L/L289V;
D286F/D287E/(del)288M/L289A;
D286F/D287E/(del)288M/L289G;
D286F/D287E/(del)288M/L281;
D286F/D287E/(del)288M/L289L;
D286F/D287E/(del)288M/L289M;
D286F/D287E/(del)288M/L289V;
D286F/D287E/(del)288V/L289A;
D286F/D287E/(del)288V/L289G;
D286F/D287E/(del)288V/L281;
D286F/D287E/(del)288V/L289L;
D286F/D287E/(del)288V/L289M;
D286F/D287E/(del)288V/L289V;
D286F/D287/(del)288A/L289A;
D286F/D287/(del)288A/L289G;
D286F/D287/(del)288A/L2891;
D286F/D287/(del)288A/L289L;
D286F/D287/(del)288A/L289M;
D286F/D287/(del)288A/L289V;
D286F/D287/(del)288G/L289A;
D286F/D287/(del)288G/L289G;
D286F/D287/(del)288G/L2891;
D286F/D287/(del)288G/L289L;
D286F/D287/(del)288G/L289M;
D286F/D287/(del)288G/L289V;
D286F/D287/(del)2881/L289A;
D286F/D287/(del)2881/L289G;
D286F/D287/(del)2881/L2891;
D286F/D287/(del)2881/L289L;
D286F/D287/(del)2881/L289M;
D286F/D287/(del)2881/L289V;
D286F/D287/(del)288L/L289A;
D286F/D287/(del)288L/L289G;
D286F/D287/(del)288L/L2891;
D286F/D287/(del)288L/L289L;
D286F/D287/(del)288L/L289M;
D286F/D287/(del)288L/L289V;
D286F/D287/(del)288M/L289A;
D286F/D287/(del)288M/L289G;
D286F/D287/(del)288M/L281;
D286F/D287/(del)288M/L289L;
D286F/D287/(del)288M/L289M;
D286F/D287/(del)288M/L289V;
D286F/D287/(del)288V/L289A;
D286F/D287/(del)288V/L289G;
D286F/D287/(del)288V/L281;
D286F/D287/(del)288V/L289L;
D286F/D287/(del)288V/L289M;
D286F/D287/(del)288V/L289V;
D286W/D287E/(del)288A/L289A;
D286W/D287E/(del)288A/L289G;
D286W/D287E/(del)288A/L2891;
D286W/D287E/(del)288A/L289L;
D286W/D287E/(del)288A/L289M;
D286W/D287E/(del)288A/L289V;
D286W/D287E/(del)288G/L289A;
D286W/D287E/(del)288G/L289G;
D286W/D287E/(del)288G/L2891;
D286W/D287E/(del)288G/L289L;
D286W/D287E/(del)288G/L289M;
D286W/D287E/(del)288G/L289V;
D286W/D287E/(del)2881/L289A;
D286W/D287E/(del)2881/L289G;
D286W/D287E/(del)2881/L2891;
D286W/D287E/(del)2881/L289L;
D286W/D287E/(del)2881/L289M;
D286W/D287E/(del)2881/L289V;
D286W/D287E/(del)288L/L289A;
D286W/D287E/(del)288L/L289G;
D286W/D287E/(del)288L/L2891;
D286W/D287E/(del)288L/L289L;
D286W/D287E/(del)288L/L289M;
D286W/D287E/(del)288L/L289V;
D286W/D287E/(del)288M/L289A;
D286W/D287E/(del)288M/L289G;
D286W/D287E/(del)288M/L281;
D286W/D287E/(del)288M/L289L;
D286W/D287E/(del)288M/L289M;
D286W/D287E/(del)288M/L289V;
D286W/D287E/(del)288V/L289A;
D286W/D287E/(del)288V/L289G;
D286W/D287E/(del)288V/L281;
D286W/D287E/(del)288V/L289L;
D286W/D287E/(del)288V/L289M;
D286W/D287E/(del)288V/L289V;
D286W/D287/(del)288A/L289A;
D286W/D287/(del)288A/L289G;
D286W/D287/(del)288A/L2891;
D286W/D287/(del)288A/L289L;
D286W/D287/(del)288A/L289M;
D286W/D287/(del)288A/L289V;
D286W/D287/(del)288G/L289A;
D286W/D287/(del)288G/L289G;
D286W/D287/(del)288G/L2891;
D286W/D287/(del)288G/L289L;
D286W/D287/(del)288G/L289M;
D286W/D287/(del)288G/L289V;
D286W/D287/(del)2881/L289A;
D286W/D287/(del)2881/L289G;
D286W/D287/(del)2881/L2891;
D286W/D287/(del)2881/L289L;
D286W/D287/(del)2881/L289M;
D286W/D287/(del)2881/L289V;
D286W/D287/(del)288L/L289A;
D286W/D287/(del)288L/L289G;
D286W/D287/(del)288L/L2891;
D286W/D287/(del)288L/L289L;
D286W/D287/(del)288L/L289M;
D286W/D287/(del)288L/L289V;
D286W/D287/(del)288M/L289A;
D286W/D287/(del)288M/L289G;
D286W/D287/(del)288M/L281;
D286W/D287/(del)288M/L289L;
D286W/D287/(del)288M/L289M;
D286W/D287/(del)288M/L289V;
D286W/D287/(del)288V/L289A;
D286W/D287/(del)288V/L289G;
D286W/D287/(del)288V/L281;
D286W/D287/(del)288V/L289L;
D286W/D287/(del)288V/L289M;
D286W/D287/(del)288V/L289V;
D286Y/D287E/(del)288A/L289A;
D286Y/D287E/(del)288A/L289G;
D286Y/D287E/(del)288A/L2891;
D286Y/D287E/(del)288A/L289L;
D286Y/D287E/(del)288A/L289M;
D286Y/D287E/(del)288A/L289V;
D286Y/D287E/(del)288G/L289A;
D286Y/D287E/(del)288G/L289G;
D286Y/D287E/(del)288G/L2891;
D286Y/D287E/(del)288G/L289L;
D286Y/D287E/(del)288G/L289M;
D286Y/D287E/(del)288G/L289V;
D286Y/D287E/(del)2881/L289A;
D286Y/D287E/(del)2881/L289G;
D286Y/D287E/(del)2881/L2891;
D286Y/D287E/(del)2881/L289L;
D286Y/D287E/(del)2881/L289M;
D286Y/D287E/(del)2881/L289V;
D286Y/D287E/(del)288L/L289A;
D286Y/D287E/(del)288L/L289G;
D286Y/D287E/(del)288L/L2891;
D286Y/D287E/(del)288L/L289L;
D286Y/D287E/(del)288L/L289M;
D286Y/D287E/(del)288L/L289V;
D286Y/D287E/(del)288M/L289A;
D286Y/D287E/(del)288M/L289G;
D286Y/D287E/(del)288M/L281;
D286Y/D287E/(del)288M/L289L;
D286Y/D287E/(del)288M/L289M;
D286Y/D287E/(del)288M/L289V;
D286Y/D287E/(del)288V/L289A;
D286Y/D287E/(del)288V/L289G;
D286Y/D287E/(del)288V/L281;
D286Y/D287E/(del)288V/L289L;
D286Y/D287E/(del)288V/L289M;
D286Y/D287E/(del)288V/L289V;
D286Y/D287/(del)288A/L289A;
D286Y/D287/(del)288A/L289G;
D286Y/D287/(del)288A/L2891;
D286Y/D287/(del)288A/L289L;
D286Y/D287/(del)288A/L289M;
D286Y/D287/(del)288A/L289V;
D286Y/D287/(del)288G/L289A;
D286Y/D287/(del)288G/L289G;
D286Y/D287/(del)288G/L2891;
D286Y/D287/(del)288G/L289L;
D286Y/D287/(del)288G/L289M;
D286Y/D287/(del)288G/L289V;
D286Y/D287/(del)2881/L289A;
D286Y/D287/(del)2881/L289G;
D286Y/D287/(del)2881/L2891;
D286Y/D287/(del)2881/L289L;
D286Y/D287/(del)2881/L289M;
D286Y/D287/(del)2881/L289V;
D286Y/D287/(del)288L/L289A;
D286Y/D287/(del)288L/L289G;
D286Y/D287/(del)288L/L2891;
D286Y/D287/(del)288L/L289L;
D286Y/D287/(del)288L/L289M;
D286Y/D287/(del)288L/L289V;
D286Y/D287/(del)288M/L289A;
D286Y/D287/(del)288M/L289G;
D286Y/D287/(del)288M/L281;
D286Y/D287/(del)288M/L289L;
D286Y/D287/(del)288M/L289M;
D286Y/D287/(del)288M/L289V;
D286Y/D287/(del)288V/L289A;
D286Y/D287/(del)288V/L289G;
D286Y/D287/(del)288V/L281;
D286Y/D287/(del)288V/L289L;
D286Y/D287/(del)288V/L289M; and
D286Y/D287/(del)288V/L289V, each with number relative to positions set forth in SEQ ID NOS: 11, 14, or 15.

7. The method of claim 3, wherein the one or more polynucleotides encode a modified α-SNAP polypeptide, wherein:
the encoded α-SNAP polypeptide comprises at least one modification corresponding to D208E, numbering corresponding by alignment with the polypeptide of SEQ ID NO: 14, or Q203K, numbering corresponding by alignment with the polypeptide of SEQ ID NO:15.

8. The method of claim 3, wherein the encoded modified α-SNAP further comprises optional amino acid replacements, including amino acid insertions or deletions, at positions 285, 286, 287, and 288, that alter α-SNAP protein interactions with NSF proteins, with numbering relative to the α-SNAP polypeptide set forth in SEQ ID NOS: 11.

9. The method of claim 1 wherein the plant cells with enhanced resistance to nematodes are produced in plants that also express wild type α-SNAP polypeptide sequences.

10. The method of claim 1, wherein the one or more polynucleotides encodes a modified NSF polypeptide, wherein:
the modified NSF polypeptide comprises one or a plurality of amino acid modifications at positions corresponding to 4 and 21 and optionally positions 25, 116, and 181, with numbering relative to the NSF polypeptide set forth in SEQ ID NOS: 17 or 18;
the modified NSF polypeptide comprises one or a plurality of amino acid modifications compared to the NSF polypeptide set forth in SEQ ID NO 17; whereby the modified NSF polypeptide comprises a sequence of amino acids that has less than 100% identity and more than 75% identity to the NSF polypeptide as set forth in SEQ ID NO 17; and
the modified NSF is a growth promoting and survival variant of the plant cell that is greater than the growth or survival of the plant cell without the NSF amino acid modification or amino acid modifications.

11. The method of claim 10, wherein the encoded modified NSF comprises amino acid modifications at positions corresponding to positions 4 and 21 by NSF numbering relative to position in the NSF polypeptide set forth in SEQ ID NOS: 17 or 18.

12. The method of claim 10, wherein the encoded modified NSF one or more polynucleotides encode a modified NSF polypeptide, wherein the modified NSF polypeptide comprises:
a modification at position R4 that is R4N, R4C, R4Q, R4S, or R4T; and
a modification at position N21 that is N21F, N21W, or N21Y, or
or a functional equivalent amino acid to the WT amino acid expressed at position 4 and 21 each by NSF numbering relative to the positions set for in SEQ ID NO: 17.

13. The method of claim 12, wherein the encoded modified NSF polypeptide comprises amino acid modifications selected from among modifications corresponding to:
R4N/N21F;
R4N/N21W;
R4N/N21Y;
R4C/N21F;
R4C/N21W;
R4C/N21Y;
R4Q/N21F;
R4Q/N21W;
R4Q/N21Y;
R4S/N21F;
R4S/N21W;
R4S/N21Y;
R4T/N21F;
R4T/N21W; and
R4T/N21Y, each with number relative to positions set forth in SEQ ID NOS: 17 or 18.

14. The method of claim 10, wherein the one or more polynucleotides encode a modified NSF polypeptide, wherein:
the encoded NSF polypeptide comprises at least one modification corresponding to R4Q and N21Y numbering with reference to the positions set forth in SEQ ID NOS: 8 or 9, and corresponding amino acids are identified by alignment with the polypeptide of SEQ ID NOS: 17 or 18.

15. The method of claim 10, wherein the encoded modified NSF further comprises optional amino acid modifications at positions 25, 116, and 181 corresponding to:
S25N;
(del)116F; and
M1811,
with numbering relative to the NSF polypeptide set forth in SEQ ID NOS: 17 or 18.

16. The method of claim 1 wherein the plant cells with enhanced resistance to nematodes are produced in the plants comprising NSF polypeptides having amino acid sequence modifications identified in Table 5.

17. The method of claim 1, wherein expression of one or more polynucleotides is increased in plant cells in the root of the plant.

18. The method of claim 1 wherein expression of one or more native polynucleotides is increased.

19. The method of claim 1, wherein an amount of an α-SNAP is decreased.

20. The method of claim 19, wherein an amount of an α-SNAP encoded by the sequence identified in SEQ ID NO: 2 or a polynucleotide with at least 75% identity thereof, or homologs or functionally conserved variants thereof, is reduced relative to an amount of an α-SNAP encoded by either of the sequences identified in SEQ ID NO: 5 and SEQ ID NO: 6 or a polynucleotide with at least 75% identity thereof, or homologs or functionally conserved variants thereof.

21. The method of claim 1, wherein expression of one or more polynucleotides encoding α-SNAP proteins, or homologs or variants thereof, or one or more polynucleotides encoding NSF proteins, or homologs or variants thereof, is increased by incorporation of a construct comprising a promoter operably linked to one or more of the polynucleotides in the plant cells.

22. The method of claim 1 wherein at least two of the recited polynucleotides have increased expression, an altered expression pattern, an altered abundance or localization of a polypeptide product of, or increased copy number.

23. The method of claim 1, wherein the plant cells comprise a nematode-resistant plant.

24. A recombinant expression construct comprising a promoter operably linked to one or more of:
(i) one or more polynucleotides encoding α-SNAP proteins, or homologs or variants thereof, or
(ii) one or more polynucleotides encoding NSF proteins, or homologs or variants thereof.

25. The construct of claim 24, comprising a polynucleotide according to SEQ ID NO: 5 or SEQ ID NO: 6, or a polynucleotide with at least 75% identity to SEQ ID NO: 5 or SEQ ID NO: 6, or a polynucleotide according to SEQ ID NO: 9, or with at least 75% identity to SEQ ID NO: 9, or homo logs or functionally conserved variants thereof.

26. The construct of claim 24, wherein the promoter is a plant promoter.

27. A nematode-resistant transgenic plant cell comprising:
(i) one or more polynucleotides encoding α-SNAP proteins, or homologs or variants thereof, or
(ii) one or more polynucleotides encoding NSF proteins, or homologs or variants thereof.

28. The transgenic plant cell of claim 27, wherein the one or more α-SNAP proteins are encoded by polynucleotides with at least 75% identity to the polynucleotides identified by SEQ ID NOS: 1-7, or comprise polypeptides with at least 75% identity to polypeptides identified by SEQ ID NOS 10-16, or homologs or variants thereof, and the one or more NSF proteins are encoded by polynucleotides with at least 75% identity to the polynucleotides identified by SEQ ID NOs: 8 and 9, or comprise polypeptides with at least 75% identity to polypeptides identified by SEQ ID Nos: 17 and 18, or homologs or variants thereof.

29. A seed comprising the transgenic plant cells of claim 27.

30. A plant grown from the seed of claim 22.

31. A transgenic plant comprising the cell of claim 27.

32. A part, progeny or asexual propagate of the transgenic plant of claim 25.

33. The transgenic plant, plant cell or seed, or part, progeny or asexual propagate thereof of claim 27, comprising NSF polypeptides having amino acid sequence modifications set forth in Table 6.

34. A method of improving growth or survival of a plant cell containing one or more Rhg1 genes conferring nematode resistance, comprising:
a) increasing expression of, altering an expression pattern of, altering a polynucleotide sequence of, altering abundance or localization of a polypeptide product of, or increasing copy number of,
(i) one or more polynucleotides encoding α-SNAP proteins, or homologs or variants thereof, or
(ii) one or more polynucleotides encoding NSF proteins, or homologs or variants thereof.

35. The method of claim 27, wherein said one or more Rhg1 genes conferring nematode resistance are identified by SEQ ID NOs: 1-7.

36. The method of claim 1, wherein the encoded NSF protein carries changes at amino acid residues 4, 21, 25, 116, with numbering relative to the NSF polypeptide set forth in SEQ ID NOS: 17 or 18, or at adjacent residues in the folded protein that interact with α-SNAP as designated in the NSF/α-SNAP/SNARE protein structure PDB ID code 3j97, or at NSF residues that are physically adjacent to the NSF residues that directly contact α-SNAP protein as identified in the NSF/α-SNAP/SNARE protein structure PDB ID code 3j97.

37. The method of claim 36, wherein modification of the amino acid residues 4, 21, 25, 116 or the other specified residues at the α-SNAP/NSF protein interface enhance growth and survival of plants expressing said α-SNAP proteins with improvements in plant resistance to cyst nematodes relative to the plant prior to this modification.

38. The method of claim 3, wherein the modified polynucleotides encode a modified α-SNAP polypeptide, wherein the modified α-SNAP polypeptide comprises:
a replacement at position E285 that is E285Q, or E285N; and
a replacement at position D286 that is D286H, or D286K, or D286R; and
a replacement at position D287 that is D287E or remains D287; and
an insertion after position 287 that is (ins)288A, (ins)288G, (ins)2881, (ins)288L, (ins)288M, or (ins)288V; and
a replacement at position L288 that is L288A, L288G, L2881, L288M, or L288V, or a
functional equivalent amino acid to the WT amino acid expressed at position 285, 286, 287, or 288, each by α-SNAP numbering relative to the positions set for in SEQ ID NO: 11.


SEQUENCE LISTING (19 sequences)

SEQ ID NO: 1 - DNA, 1311 bp, Glycine max
atgtctccggccgccggagtcagcgtccccctcctgggggattccaaaggaacgccgccgccggcttccgtccccggcgcggtgttcaacgtggccaccagcatagtcggcgccggaatcatgtcgattccggcgatcatgaaggttctcggcgtagttcccgctttcgcgatgattctcgtggtggccgtgctggcggaactgtccgtggacttcctgatgcggttcacgcactccggcgaaacgacgacgtacgctggcgtcatgagggaggcgttcggatcgggtggagcattggccgcgcaagtttgcgtcatcatcaccaacgttgggggtttaattctctaccttatcatcatcggagatgtgctatctggaaagcaaaatggaggggaagtgcatttgggcattttgcaacagtggtttggaattcactggtggaattcccgggaatttgctttgcttttcaccttggtctttgttatgcttccattggtattgtacaaacgtgtagagtccttgaagtacagctctgcagtgtcaactcttcttgcagtggcatttgttggcatatgttgtgggttggctatcacagctctggtgcaaggaaaaacacaaactcctagattgtttcctcggctagactaccaaacctcattctttgatctgttcactgcagttcctgttgttgtcacagccttcacatttcactttaatgtgcaccccattgggtttgagcttgccaaggcatcccaaatgacaacagcagttcgattagcattattgctttgtgctgtgatctaccttgcaataggcttatttgggtacatgttatttggggattcaacccagtcagacattctcatcaattttgaccagaatgctggttcagcagttggttccttgctcaatagtttggtccgtgtaagctatgccctccacatcatgctggtgtttcctctcttgaacttctctttgagaaccaacatagatgaagttctcttccctaagaagcctatgctagccacagacaacaaaagatttatgatcctcactctggtgctgcttgtattctcctaccttgcagctatagcaatcccagatatttggtacttctttcagttcctgggatcctcatccgcagtgtgccttgccttcattttccccggctctattgttttaagggatgttaaaggtatatcaacgagaagagacaaaattattgcactgataatgattatactagctgtggttacaagtgtgcttgccatttccaccaacatatataatgcttttagtagcaagtcataa

SEQ ID NO: 2 - DNA, 870 bp, Glycine max
atggccgatcagttatcgaagggagaggaattcgagaaaaaggctgagaagaagctcagcggttggggcttgtttggctccaagtatgaagatgccgccgatctcttcgataaagccgccaattgcttcaagctcgccaaatcatgggacaaggctggagcgacatacctgaagttggcaagttgtcatttgaagttggaaagcaagcatgaagctgcacaggcccatgtcgatgctgcacattgctacaaaaagactaatataaacgagtctgtatcttgcttagaccgagctgtaaatcttttctgtgacattggaagactctctatggctgctagatatttaaaggaaattgctgaattgtacgagggtgaacagaatattgagcaggctcttgtttactatgaaaaatcagctgatttttttcaaaatgaagaagtgacaacttctgcgaaccaatgcaaacaaaaagttgcccagtttgctgctcagctagaacaatatcagaagtcgattgacatttatgaagagatagctcgccaatccctcaacaataatttgctgaagtatggagttaaaggacaccttcttaatgctggcatctgccaactctgtaaagaggacgttgttgctataaccaatgcattagaacgatatcaggaactggatccaacattttcaggaacacgtgaatatagattgttggcggacattgctgctgcaattgatgaagaagatgttgcaaagtttactgatgttgtcaaggaatttgatagtatgacccctctggattcttggaagaccacacttctcttaagggtgaaggaaaagctgaaagccaaagaacttgaggaggatgatcttacttga

SEQ ID NO: 3 - DNA, 324 bp, Glycine max
atgcgcatgctcaccggcgactccgccgccgacaactccttccgattcgttccgcagtccatcgccgccttcggctccaccgtcatcgtcgagggctgcgactccgcccgcaacattgcctgggtccacgcctggaccgtcactgatgggatgatcactcaaatcagagagtacttcaacaccgccctcaccgtcactcgcatccacgattccggcgagattgttccggccagatccggcgccggccgtttgccctgcgtctgggagagcagcgtctccggtcgggtcgggaaatccgtccccggtttggttctcgcaatataa

SEQ ID NO: 4 - DNA, 1668 bp, Glycine max
atggtttcggttgatgatgggattgtgaatcccaatgatgaaattgagaaatctaacgggagtaaagtgaatgagtttgcatctatggatatttcagcaactcaaaaatcatatctgaacagtgaagatcctcagagaaggcttcagggaaccttaataagttcttctgttactaataggataaactttcttaaatttggttctgcatctgccaaattcaaaaggcttgctactgagagagaccaggtttctatatctgtgccttctcctcgttcaaagagcctaagatcacgtttcagtggcatgtttgctcagaaacttgactgggcttcagtcaagaaaatgtgcatggaatggattagaaatccagtgaacatggccctttttgtgtggatcatttgtgtcgcggtttcgggtgctattctgttccttgtcatgacaggcatgttgaatggtgtgctaccaagaaagtctaagagaaatgcatggtttgaagtaaacaaccaaatactcaatgcagtgtttacactcatgtgtttgtaccaacaccctaagagattctaccaccttgttcttctgaccagatgaagaccaaatgacatctctagccttaggaaggtatattgcaagaatgtcacttacaagccccatgagtggacacatatgatggtagttgtcattctccttcatgttaactgttttgctcaatatgcactttgtggtctaaacttagggtataaaaggtccgagagacctgccattggagttggaatatgcatatcttttgcaattgctggtttgtacaccattcttagcccacttgggaaggactatgattgtgagatggatgaagaagcacaggttcaaattacagcttctcaagggaaagagcagctgagagagaaaccaactgagaagaaatattcatttgcatccaaagatcaacaaagggttgttgaaaatagaccaaagtggagtggaggaatacttgacatttggaacgatatttccttagcatatctctcacttttctgcaccttttgtgtgcttgggtggaatatgaagaggcttggctttggaaacatgtatgttcacattgccatttttatgctgttctgtatggctcctttctggatttttcttttggcttccgttaacatagatgatgacaatgttaggcaggctctagcagctgttggaatcattctttgttttcttggtttattgtatggtggattttggaggatccaaatgagaaagaggttcaatttaccagcctatgacttctgttttggcaaaccttcagcttctgattgcacactttggctaccctgttgctggtgctctctcgctcaagaagcgcgtaccaggaataactatgatcttgtagaagataaattctcaaggaaagaaactgatactagtgatcaaccatcaatttcacctttggctcgtgaagatgtagtgtcaaccagatctggcacaagttctcctatgggtagcactagcaactcttccccttatatgatgaaaacatctagttctccaaattcaagcaatgtcttaaagggatattacagtccagataagatgctatcaactttgaatgaagacaattgtgaaagaggtcaagatggaacaatgaaccccttatatgcacaaaaataa

SEQ ID NO: 5 - DNA, 873 bp, Glycine max
atggccgatcagttatcgaagggagaggaattcgagaaaaaggctgagaagaagctcagcggttggggcttgtttggctccaagtatgaagatgccgccgatctcttcgataaagccgccaattgcttcaagctcgccaaatcatgggacaaggctggagcgacatacctgaagttggcaagttgtcatttgaagttggaaagcaagcatgaagctgcacaggcccatgtcgatgctgcacattgctacaaaaagactaatataaacgagtctgtatcttgcttagaccgagctgtaaatcttttctgtgacattggaagactctctatggctgctagatatttaaaggaaattgctgaattgtacgagggtgaacagaatattgagcaggctcttgtttactatgaaaaatcagctgatttttttcaaaatgaagaagtgacaacttctgcgaaccaatgcaaacaaaaagttgcccagtttgctgctcagctagaacaatatcagaagtcgattgacatttatgaagagatagctcgccaatccctcaacaataatttgctgaagtatggagttaaaggacaccttcttaatgctggcatctgcaaactctgtaaagaggacgttgttgctataaccaatgcattagaacgatatcaggaactggatccaacattttcaggaacacgtgaatatagattgttggcggacattgctgctgcaattgatgaagaagatgttgcaaagtttactgatgttgtcaaggaatttgatagtatgacccctctggattcttggaagaccacacttctcttaagggtgaaggaaaagctgaaagccaaagaacttgagcagcatgaggctattacttga

SEQ ID NO: 6 - DNA, 873 bp, Glycine max
atggccgatcagttatcgaagggagaggaattcgagaaaaaggctgagaagaagctcagcggttggggcttgtttggctccaagtatgaagatgccgccgatctcttcgataaagccgccaattgcttcaagctcgccaaatcatgggacaaggctggagcgacatacctgaagttggcaagttgtcatttgaagttggaaagcaagcatgaagctgcacaggcccatgtcgatgctgcacattgctacaaaaagactaatataaacgagtctgtatcttgcttagaccgagctgtaaatcttttctgtgacattggaagactctctatggctgctagatatttaaaggaaattgctgaattgtacgagggtgaacagaatattgagcaggctcttgtttactatgaaaaatcagctgatttttttcaaaatgaagaagtgacaacttctgcgaaccaatgcaaacaaaaagttgcccagtttgctgctcagctagaacaatatcagaagtcgattgacatttatgaagagatagctcgccaatccctcaacaataatttgctgaagtatggagttaaaggacaccttcttaatgctggcatctgccaactctgtaaagaggaggttgttgctataaccaatgcattagaacgatatcaggaactggatccaacattttcaggaacacgtgaatatagattgttggcggacattgctgctgcaattgatgaagaagatgttgcaaagtttactgatgttgtcaaggaatttgatagtatgacccctctggattcttggaagaccacacttctcttaagggtgaaggaaaagctgaaagccaaagaacttgaggagtatgaggttattacttga

SEQ ID NO: 7 - DNA, 837 bp, Glycine max
atggccgatcagttatcgaagggagaggaattcgagaaaaaggctgagaagaagctcagcggttggggcttgtttggctccaagtatgaagatgccgccgatctcttcgataaagccgccaattgcttcaagctcgccaaatcatgggacaaggctggagcgacatacctgaagttggcaagttgtcatttgaagttggaaagcaagcatgaagctgcacaggcccatgtcgatgctgcacattgctacaaaaagactaatataaacgagtctgtatcttgcttagaccgagctgtaaatcttttctgtgacattggaagactctctatggctgctagatatttaaaggaaattgctgaattgtacgagggtgaacagaatattgagcaggctcttgtttactatgaaaaatcagctgatttttttcaaaatgaagaagtgacaacttctgcgaaccaatgcaaacaaaaagttgcccagtttgctgctcagctagaacaatatcagaagtcgattgacatttatgaagagatagctcgccaatccctcaacaataatttgctgaagtatggagttaaaggacaccttcttaatgctggcatctgccaactctgtaaagaggaggaactggatccaacattttcaggaacacgtgaatatagattgttggcggacattgctgctgcaattgatgaagaagatgttgcaaagtttactgatgttgtcaaggaatttgatagtatgacccctctggattcttggaagaccacacttctcttaagggtgaaggaaaagctgaaagccaaagaacttgaggagtatgaggttattacttga

SEQ ID NO: 8 - DNA, 2241 bp, Glycine max
atggcgagtcggttcgggttatcgtcttcgtcttcctctgcgtccagcatgagagttaccaacacgcccgcgagcgacctcgccctcaccaacctcgccttctgttccccctccgatctccgcaatttcgccgtccctggccacaataacctctacctcgccgccgtcgccgattccttcgtcttatctctctctgctcatgacaccataggcagcggtcagattgcgttgaatgccgttcaacgccggtgtgccaaagtttcttccggtgattccgtacaagtgagccgatttgtgccgcctgaagatttcaacctcgcactgctaactcttgaattggaatttgttaaaaaggggagtaagagtgagcagattgatgctgttctactggctaagcaacttcgtaagagatttatgaaccaggttatgactgtggggcagaaagtattatttgagtatcacggaaataattatagctttactgtcagtaatgctgctgttgagggccaagaaaagtctaattctcttgaaagagggatgatttcagatgacacatacattgtttttgaaacatcacgtgatagtggaattaagattgtcaatcaacgagagggtgccactagcaacattttcaagcagaaagaatttaaccttcagtcactgggtattggtggcctgagtgcagaatttgcagatatatttcgaagagcttttgcctctcgtgttttcccaccccatgtgacatctaaattaggaatcaagcatgtgaagggcatgcttctttatgggcctcctggaactggaaagacacttatggcacgccaaattggaaaaattttgaatgggaaggaacccaagattgtaaatggccctgaagttttgagcaaatttgttggtgaaactgaaaagaatgtgagagacctttttgctgatgctgaacaggatcagaggacccgaggggatgaaagtgatttgcatgttataatctttgatgaaattgatgctatttgcaagtcaagaggttcaactcgagatggtactggagttcatgatagtattgtaaatcagcttcttactaagatagatggtgtggagtcactaaataatgttttacttattggaatgactaacagaaaggacatgcttgatgaagctctcttaagaccagggaggttggaagtccaggttgagataagccttcctgatgaaaatggtcgattgcaaattcttcaaatccatactaacaaaatgaaagagaattcttttctagctgctgatgtgaaccttcaagagcttgctgctcgaacgaaaaactacagtggtgcagaacttgaaggtgttgtgaaaagtgctgtctcatatgctttaaatagacaattgagtctagaggatctcactaagccagtggaggaagagaacattaaggttacaatggatgactttttgaatgcactccacgaagttacttccgcatttggagcttcaactgatgatcttgaaagatgcagactccatggcatggttgagtgtggtgatcgacataagcacatttatcaaagagcaatgctacttgtggagcaagttaaagtgagtaaaggaagcccacttgtcacttgtctcctggaaggttcccgtggcagtggtaaaactgcactttcagctactgttggtatcgacagcgacttcccatacgtcaagatagtttcagctgaatcaatgattggtctacatgagagcaccaaatgtgcacagattattaaggtttttgaggatgcatacaagtcaccattgagtgtcatcattcttgatgacattgagagattattggagtatgtgcccattggtcctcgattttcaaacttgatttctcagacactgctggttctgctcaaacggcttcctccaaaggggaaaaaactaatggttattggcacaacaagtgaactagatttcttggaatcaattggattttgtgataccttctctgttacttaccatattcctaccttgaacacaacggatgcaaagaaggtcctagaacagttgaatgtgtttactgatgaagatattgattctgctgcagaggcgttgaatgatatgcctatcaggaaactatacatgttgatcgagatggcagcgcaaggggagcatggtggatctgcagaagccatcttttctggcaaagagaagattagtatcgctcatttctatgattgcctccaggatgttgttaggttataa

SEQ ID NO: 9 - DNA, 2244 bp, Glycine max
atggcgagtcagttcgggttatcgtcttcgtcttcctctgcgtccagcatgagagttacctacacgcccgcgaacgacctcgccctcaccaacctcgccttctgttccccctccgatctccgcaatttcgccgtccctggccacaataacctctacctcgccgccgtcgccgattccttcgtcttatctctctctgctcatgacaccataggcagcggtcagattgcgttgaatgccgttcaacgccggtgtgccaaagtttcttccggtgattccgtacaagtgagccgatttgtgccgcctgaagatttcaacctcgcactgctaactcttgaattggaattttttgttaaaaaggggagtaagagtgagcagattgatgctgttctactggctaagcaacttcgtaagagatttatgaaccaggttatgactgtggggcagaaagtattatttgagtatcacggaaataattatagctttactgtcagtaatgctgctgttgagggccaagaaaagtctaattctcttgaaagagggattatttcagatgacacatacattgtttttgaaacatcacgtgatagtggaattaagattgtcaatcaacgagagggtgccactagcaacattttcaagcagaaagaatttaaccttcagtcactgggtattggtggcctgagtgcagaatttgcagatatatttcgaagagcttttgcctctcgtgttttcccaccccatgtgacatctaaattagggatcaagcatgtgaagggcatgcttctttatgggcctcctggaactggaaagacacttatggcacgccaaattggaaaaattttgaatgggaaggaacccaagattgtaaatggccctgaagttttgagcaaatttgttggtgaaactgaaaagaatgtgagagacctttttgctgatgctgaacaggatcagaggacccgaggggatgaaagtgatttgcatgttataatctttgatgaaattgatgctatttgcaagtcaagaggttcaactcgagatggtactggagttcatgatagtattgtaaatcagcttcttactaagatagatggtgtggagtcactaaataatgttttacttattggaatgactaacagaaaggacatgcttgatgaagctctcttaagaccagggaggttggaagtccaggttgagataagccttcctgatgaaaatggtcgattgcaaattcttcaaattcatactaacaaaatgaaagagaattcttttctagctgctgatgtgaaccttcaagagcttgctgctcgaacgaaaaactacagtggtgcagaacttgaaggtgttgtgaaaagtgctgtctcatatgctttaaatagacaattgagtctagaggatctcactaagccagtggaggaagagaacattaaggttacaatggatgactttttgaatgcactccacgaagttacttccgcatttggagcttcaactgatgatcttgaaagatgcagactccatggcatggttgagtgtggtgatcgacataagcacatttatcaaagagcaatgctacttgtggagcaagttaaagtgagtaaaggaagcccacttgtcacttgtctcctggaaggttcccgtggcagtggtaaaactgcactttcagctactgttggtatcgacagcgacttcccatacgtcaagatagtttcagctgaatcaatgattggtctacatgagagcaccaaatgtgcacagattattaaggtttttgaggatgcatacaagtcaccattgagtgtcatcattcttgatgacattgagagattattggagtatgtgcccattggtcctcgattttcaaacttgatttctcagacactgctggttctgctcaaacggcttcctccaaaggggaaaaaactcatggttattggcacaacaagtgaactagatttcttggaatcaattggattttgtgataccttctctgttacttaccatattcctaccttgaacacaacggatgcaaagaaggtcctagaacagttgaatgtgtttactgatgaagatattgattctgctgcagaggcgttgaatgatatgcctatcaggaaactatacatgttgatcgagatggcagcgcaaggggagcatggtggatctgcagaagccatcttttctggcaaagagaagattagtatcgctcacttctatgattgcctccaggatgttgttaggttatga

SEQ ID NO: 10 - protein, 436 aa, Glycine max
MSPAAGVSVPLLGDSKGTPPPASVPGAVFNVATSIVGAGIMSIPAIMKVLGVVPAFAMILVVAVLAELSVDFLMRFTHSGETTTYAGVMREAFGSGGALAAQVCVIITNVGGLILYLIIIGDVLSGKQNGGEVHLGILQQWFGIHWWNSREFALLFTLVFVMLPLVLYKRVESLKYSSAVSTLLAVAFVGICCGLAITALVQGKTQTPRLFPRLDYQTSFFDLFTAVPVVVTAFTFHFNVHPIGFELAKASQMTTAVRLALLLCAVIYLAIGLFGYMLFGDSTQSDILINFDQNAGSAVGSLLNSLVRVSYALHIMLVFPLLNFSLRTNIDEVLFPKKPMLATDNKRFMILTLVLLVFSYLAAIAIPDIWYFFQFLGSSSAVCLAFIFPGSIVLRDVKGISTRRDKIIALIMIILAVVTSVLAISTNIYNAFSSKS

SEQ ID NO: 11 - protein, 289 aa, Glycine max
MADQLSKGEEFEKKAEKKLSGWGLFGSKYEDAADLFDKAANCFKLAKSWDKAGATYLKLASCHLKLESKHEAAQAHVDAAHCYKKTNINESVSCLDRAVNLFCDIGRLSMAARYLKEIAELYEGEQNIEQALVYYEKSADFFQNEEVTTSANQCKQKVAQFAAQLEQYQKSIDIYEEIARQSLNNNLLKYGVKGHLLNAGICQLCKEDVVAITNALERYQELDPTFSGTREYRLLADIAAAIDEEDVAKFTDVVKEFDSMTPLDSWKTTLLLRVKEKLKAKELEEDDLT

SEQ ID NO: 12 - protein, 80 aa, Glycine max
MRMLTGDSAADNSFRFVPQSIAAFGSTVIVEGCDSARNIAWVHAWTVTDGMITQIREYFNTALTVTRIHDSGEIVPARSG

SEQ ID NO: 13 - protein, 536 aa, Glycine max
MVSVDDGIVNPNDEIEKSNGSKVNEFASMDISATQKSYLNSEDPQRRLQGTLISSSVTNRINFLKFGSASAKFKRLATERDQVSISVPSPRSKSLRSRFSGMFAQKLDWASVKKMCMEWIRNPVNMALFVWIICVAVSGAILFLVMTGMLNGVLPRKSKRNAWFEVNNQILNAVFTLIPNDISSLRKVYCKNVTYKPHEWTHMMVVVILLHVNCFAQYALCGLNLGYKRSERPAIGVGICISFAIAGLYTILSPLGKDYDCEMDEEAQVQITASQGKEQLREKPTEKKYSFASKDQQRVVENRPKWSGGILDIWNDISLAYLSLFCTFCVLGWNMKRLGFGNMYVHIAIFMLFCMAPFWIFLLASVNIDDDNVRQALAAVGIILCFLGLLYGGFWRIQMRKRFNLPAYDFCFGKPSASDCTLWLPCCWCSLAQEARTRNNYDLVEDKFSRKETDTSDQPSISPLAREDVVSTRSGTSSPMGSTSNSSPYMMKTSSSPNSSNVLKGYYSPDKMLSTLNEDNCERGQDGTMNPLYAQK

SEQ ID NO: 14 - protein, 290 aa, Glycine max
MADQLSKGEEFEKKAEKKLSGWGLFGSKYEDAADLFDKAANCFKLAKSWDKAGATYLKLASCHLKLESKHEAAQAHVDAAHCYKKTNINESVSCLDRAVNLFCDIGRLSMAARYLKEIAELYEGEQNIEQALVYYEKSADFFQNEEVTTSANQCKQKVAQFAAQLEQYQKSIDIYEEIARQSLNNNLLKYGVKGHLLNAGICKLCKEDVVAITNALERYQELDPTFSGTREYRLLADIAAAIDEEDVAKFTDVVKEFDSMTPLDSWKTTLLLRVKEKLKAKELEQHEAIT

SEQ ID NO: 15 - protein, 290 aa, Glycine max
MADQLSKGEEFEKKAEKKLSGWGLFGSKYEDAADLFDKAANCFKLAKSWDKAGATYLKLASCHLKLESKHEAAQAHVDAAHCYKKTNINESVSCLDRAVNLFCDIGRLSMAARYLKEIAELYEGEQNIEQALVYYEKSADFFQNEEVTTSANQCKQKVAQFAAQLEQYQKSIDIYEEIARQSLNNNLLKYGVKGHLLNAGICQLCKEEVVAITNALERYQELDPTFSGTREYRLLADIAAAIDEEDVAKFTDVVKEFDSMTPLDSWKTTLLLRVKEKLKAKELEEYEVIT

SEQ ID NO: 16 - protein, 278 aa, Glycine max
MADQLSKGEEFEKKAEKKLSGWGLFGSKYEDAADLFDKAANCFKLAKSWDKAGATYLKLASCHLKLESKHEAAQAHVDAAHCYKKTNINESVSCLDRAVNLFCDIGRLSMAARYLKEIAELYEGEQNIEQALVYYEKSADFFQNEEVTTSANQCKQKVAQFAAQLEQYQKSIDIYEEIARQSLNNNLLKYGVKGHLLNAGICQLCKEEELDPTFSGTREYRLLADIAAAIDEEDVAKFTDVVKEFDSMTPLDSWKTTLLLRVKEKLKAKELEEYEVIT

SEQ ID NO: 17 - protein, 746 aa, Glycine max
MASRFGLSSSSSSASSMRVTNTPASDLALTNLAFCSPSDLRNFAVPGHNNLYLAAVADSFVLSLSAHDTIGSGQIALNAVQRRCAKVSSGDSVQVSRFVPPEDFNLALLTLELEFVKKGSKSEQIDAVLLAKQLRKRFMNQVMTVGQKVLFEYHGNNYSFTVSNAAVEGQEKSNSLERGMISDDTYIVFETSRDSGIKIVNQREGATSNIFKQKEFNLQSLGIGGLSAEFADIFRRAFASRVFPPHVTSKLGIKHVKGMLLYGPPGTGKTLMARQIGKILNGKEPKIVNGPEVLSKFVGETEKNVRDLFADAEQDQRTRGDESDLHVIIFDEIDAICKSRGSTRDGTGVHDSIVNQLLTKIDGVESLNNVLLIGMTNRKDMLDEALLRPGRLEVQVEISLPDENGRLQILQIHTNKMKENSFLAADVNLQELAARTKNYSGAELEGVVKSAVSYALNRQLSLEDLTKPVEEENIKVTMDDFLNALHEVTSAFGASTDDLERCRLHGMVECGDRHKHIYQRAMLLVEQVKVSKGSPLVTCLLEGSRGSGKTALSATVGIDSDFPYVKIVSAESMIGLHESTKCAQIIKVFEDAYKSPLSVIILDDIERLLEYVPIGPRFSNLISQTLLVLLKRLPPKGKKLMVIGTTSELDFLESIGFCDTFSVTYHIPTLNTTDAKKVLEQLNVFTDEDIDSAAEALNDMPIRKLYMLIEMAAQGEHGGSAEAIFSGKEKISIAHFYDCLQDVVRL

SEQ ID NO: 18 - protein, 747 aa, Glycine max
MASQFGLSSSSSSASSMRVTYTPANDLALTNLAFCSPSDLRNFAVPGHNNLYLAAVADSFVLSLSAHDTIGSGQIALNAVQRRCAKVSSGDSVQVSRFVPPEDFNLALLTLELEFFVKKGSKSEQIDAVLLAKQLRKRFMNQVMTVGQKVLFEYHGNNYSFTVSNAAVEGQEKSNSLERGIISDDTYIVFETSRDSGIKIVNQREGATSNIFKQKEFNLQSLGIGGLSAEFADIFRRAFASRVFPPHVTSKLGIKHVKGMLLYGPPGTGKTLMARQIGKILNGKEPKIVNGPEVLSKFVGETEKNVRDLFADAEQDQRTRGDESDLHVIIFDEIDAICKSRGSTRDGTGVHDSIVNQLLTKIDGVESLNNVLLIGMTNRKDMLDEALLRPGRLEVQVEISLPDENGRLQILQIHTNKMKENSFLAADVNLQELAARTKNYSGAELEGVVKSAVSYALNRQLSLEDLTKPVEEENIKVTMDDFLNALHEVTSAFGASTDDLERCRLHGMVECGDRHKHIYQRAMLLVEQVKVSKGSPLVTCLLEGSRGSGKTALSATVGIDSDFPYVKIVSAESMIGLHESTKCAQIIKVFEDAYKSPLSVIILDDIERLLEYVPIGPRFSNLISQTLLVLLKRLPPKGKKLMVIGTTSELDFLESIGFCDTFSVTYHIPTLNTTDAKKVLEQLNVFTDEDIDSAAEALNDMPIRKLYMLIEMAAQGEHGGSAEAIFSGKEKISIAHFYDCLQDVVRL

SEQ ID NO: 19 - protein, 740 aa, Cricetulus griseus
MAGRSMQAARCPTDELSLSNCAVVSEKDYQSGQHVIVRTSPNHKYIFTLRTHPSVVPGSVAFSLPQRKWAGLSIGQEIEVALYSFDKAKQCIGTMTIEIDFLQKKNIDSNPYDTDKMAAEFIQQFNNQAFSVGQQLVFSFNDKLFGLLVKDIEAMDPSILKGEPASGKRQKIEVGLVVGNSQVAFEKAENSSLNLIGKAKTKENRQSIINPDWNFEKMGIGGLDKEFSDIFRRAFASRVFPPEIVEQMGCKHVKGILLYGPPGCGKTLLARQIGKMLNAREPKVVNGPEILNKYVGESEANIRKLFADAEEEQRRLGANSGLHIIIFDEIDAICKQRGSMAGSTGVHDTVVNQLLSKIDGVEQLNNILVIGMTNRPDLIDEALLRPGRLEVKMEIGLPDEKGRLQILHIHTARMRGHQLLSADVDIKELAVETKNFSGAELEGLVRAAQSTAMNRHIKASTKVEVDMEKAESLQVTRGDFLASLENDIKPAFGTNQEDYASYIMNGIIKWGDPVTRVLDDGELLVQQTKNSDRTPLVSVLLEGPPHSGKTALAAKIAEESNFPFIKICSPDKMIGFSETAKCQAMKKIFDDAYKSQLSCVVVDDIERLLDYVPIGPRFSNLVLQALLVLLKKAPPQGRKLLIIGTTSRKDVLQEMEMLNAFSTTIHVPNIATGEQLLEALELLGNFKDKERTTIAQQVKGKKVWIGIKKLLMLIEMSLQMDPEYRVRKFLALLREEGASP